(19) United States
(12) Patent Application Publication
(10) Pub. No.: US 2026/0134967 A1
(43) Pub. Date: May 14, 2026

(54) METHODS TO MEASURE ANTIGEN-SPECIFIC T CELL CLONES AND USES THEREOF

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Vinod P. Balachandran, New York, NY (US); Benjamin Dylan Greenbaum, New York, NY (US); Zachary Sethna, New York, NY (US); Luis Rojas, New York, NY (US)

(21) Appl. No.: 19/116,299

(22) PCT Filed: Sep. 29, 2023
(86) PCT No.: PCT/US2023/075539

§ 371 Date: Mar. 27, 2025

Related U.S. Application Data
(60) Provisional application No. 63/412,065, filed on Sep. 30, 2022.

(51) Int. Cl. G16H 20/17; G16B 20/20; G16H 50/70

(52) U.S. Cl. CPC G16H 20/17; G16B 20/20; G16H 50/70

(57) ABSTRACT

The present disclosure provides computational systems and methods for identifying and monitoring antigen-specific T cell clones that increase or decrease in response to immunotherapies, and method for using the same to customize therapeutic interventions in cancer patients.

### CROSS-REFERENCE TO RELATED APPLICATIONS

This application claims the benefit of and priority to U.S. Provisional Patent Application No. 63/412,065 filed Sep. 30, 2022, the entire contents of which are incorporated herein by reference.

### TECHNICAL FIELD

The present disclosure provides computational systems and methods for identifying and monitoring antigen-specific T cell clones that increase or decrease in response to immunotherapies, and method for using the same to customize therapeutic interventions in cancer patients.

### BACKGROUND

Pancreas ductal adenocarcinoma (PDAC) is the third leading cause of cancer death in the United States, and the seventh leading cause of cancer death worldwide. Though mortality has decreased for nearly all other common cancers, survival rates for PDAC have stagnated for over 60 years. Five-year overall survival (OS) for patients with PDAC remains dismal at <10%. Multi-agent chemotherapy is the standard of care for the 85% of patients who present with distant metastases or surgically unresectable tumors, but confers a median survival of only <18 months. Surgery and adjuvant combination chemotherapy is the standard in the 15% of patients with surgically resectable tumors. However, nearly 80% of these patients recur at ˜14 months, and their 5-year OS is only <30%. Radiation, biologic, and targeted therapies are also ineffective.

### SUMMARY OF THE PRESENT TECHNOLOGY

Aspects of the present disclosure are directed to systems and methods for determining a likelihood of responsiveness to an immunotherapy in a subject suffering from cancer. One or more processors may receive a dataset identifying a plurality of sequence reads. Each sequence read of the plurality sequence reads may correspond to a respective T-cell receptor beta locus (TRB) sequence or T-cell receptor alpha locus (TRA) sequence derived from T-cell receptor (TCR) encoding polynucleotides present in a first plurality of T cell clones in a first biological sample from the subject administered with an immunotherapy. The first plurality of T cell clones may belong to a plurality of clonotypes. The one or more processors may, for each clonotype of the plurality of clonotypes: identify a first distribution of the first plurality of T cell clones belonging to the clonotype in the first biological sample using the plurality of sequence reads from the dataset; compare the first distribution of the first plurality of T cell clones belonging to the clonotype with a second distribution of a second plurality of T cells belonging to the clonotype in a reference biological sample; generate, a first significance value indicating an expansion of T cell clones of the clonotype within the first biological sample based on comparing the first distribution with the second distribution in accordance with a Fisher's exact test; and adjust the first significance value for the clonotype based on a number of clonotypes within the plurality of clonotypes in the first biological sample and the reference biological sample, to generate a second significance value. The one or more processors may determine a score indicating a likelihood of responsiveness to the immunotherapy in the subject based on the second significance value for at least one clonotype of the plurality of clonotypes. The one or more processors may store, using one or more data structure, an association between the score indicating the likelihood of responsiveness and the subject.

In some embodiments, the one or more processors may identify the subject as a responder to the immunotherapy based on the score satisfying a threshold. In some embodiments, the one or more processors may provide an instruction to continue administration of the immunotherapy to the subject, responsive to identifying the subject as the responder. In some embodiments, the one or more processors may identify the subject as not a responder to the immunotherapy based on the score not satisfying a threshold. In some embodiments, the one or more processors may provide an instruction to discontinue administration of the immunotherapy to the subject, responsive to identifying the subject as the non-responder.

In some embodiments, the one or more processors may determine a threshold to compare against the score based on a frequency of the second plurality of T cells corresponding to each clonotype of the plurality of clonotypes in the reference biological sample. In some embodiments, the one or more processors may generate, for each clonotype of the plurality of clonotypes, the significance value indicating a two-fold expansion of T cell clones. In some embodiments, the one or more processors may, rescale, for each clonotype of the plurality of clonotypes, the first distribution of the first plurality of T cell clones of the clonotype based on a difference between (i) a factor of a number of the plurality of clonotypes in the first biological sample and (ii) a number of the first plurality of T cell clones belonging to the clonotype.

In some embodiments, the one or more processors may rescale, for each clonotype of the plurality of clonotypes, the second distribution of the second plurality of T cells of the clonotype to compare against the first distribution of T cells of the clonotype, based on a difference between (i) a number of the plurality of clonotypes in the reference biological sample and (ii) a number of the second plurality of T cell clones belonging to the clonotype. In some embodiments, the one or more processors may adjust the significance value for each clonotype of the plurality of clonotypes in accordance with a Bonferroni correction based on a number of the plurality of clonotypes in both the first biological sample and the reference biological sample.

In some embodiments, the one or more processors may determine that the first distribution of the plurality of T cell clones belonging to at least one clonotype of the plurality of clonotypes does not satisfy a threshold. In some embodiments, the one or more processors may assign the first distribution of the plurality of T cell clones belonging to the at least one clonotype to a value based on a factor of a number of the plurality of clonotypes in the first biological sample. In some embodiments, the one or more processors may remove, from the plurality of sequence reads, a subset of sequence reads determined as non-productive based on an identification of recombined TCR alpha CDR3 nucleotide sequences or TCR beta CDR3 nucleotide sequences from silenced alleles.

In some embodiments, the one or more processors identify first distribution of T cell clones further comprises identifying, for each clonotype of the plurality of clonotypes. The first distribution of the first plurality of T cell clones may correspond to the clonotype based on a respective permutation of variable (V) gene, a joining (J) gene, and a nucleotide CDR3 sequence. In some embodiments, the reference biological sample may be obtained from the subject prior to the administration of the immunotherapy. In other embodiments, the reference biological sample may be obtained from a cancer patient that does not receive the immunotherapy. In some embodiments, the first biological sample and the second biological sample may be the same type. In some embodiments, the reference biological sample may be the same type as the first biological sample and the second biological sample. In some embodiments, the immunotherapy may include an anti-cancer vaccine, monoclonal antibody-based immunotherapy, or an immune checkpoint inhibitor. Additionally or alternatively, in some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. Additionally or alternatively, in some embodiments, the first biological sample may be obtained at least 1 week, at least 2 weeks, at least 3 weeks, 4 at least weeks, or at least 5 weeks after the immunotherapy has been administered to the subject.

Aspects of the present disclosure are directed to systems and methods for monitoring responsiveness to at least one type of immunotherapy in a subject suffering from cancer. One or more processors may receive, at a first time prior to an administration of the first immunotherapy to the subject, a first dataset identifying a first plurality of sequence reads. Each sequence read of the first plurality sequence reads may correspond to a respective T-cell receptor beta locus (TRB) sequence or T-cell receptor alpha locus (TRA) sequence derived from T-cell receptor (TCR) encoding polynucleotides present in a first plurality of T cell clones in a first biological sample from the subject. The first plurality of T cell clones may belong to a plurality of clonotypes. The one or more processors may identify, for each of the plurality of clonotypes, a first distribution of the first plurality of T cell clones belonging to the clonotype in the first biological sample using the first plurality of sequence reads from the first dataset. The one or more processors may receive, at a second time subsequent to the administration of the first immunotherapy to the subject, a second dataset identifying a second plurality of sequence reads. Each sequence read of the second plurality sequence reads may correspond to a respective TRB sequence or TRA sequence derived from TCR encoding polynucleotides present in a second plurality of T cell clones in a second biological sample from the subject.

The one or more processors may, for each clonotype of the plurality of clonotypes: identify a second distribution of the second plurality of T cell clones belonging to the clonotype in the second biological sample using the second plurality of sequence reads from the dataset; compare the first distribution of the first plurality of T cell clones with the second distribution of the second plurality of T cells; generate a first significance value indicating an expansion of T cell clones of the clonotype within the second biological sample based on comparing the first distribution with the second distribution in accordance with a Fisher's exact test; and adjust the first significance value for the clonotype based on a number of clonotypes within a plurality of clonotypes in the first biological sample and the second biological sample, to generate a second significance value. The one or more processors may determine a score identifying a degree of responsiveness to the first immunotherapy in the subject over the first time and the second time based on the second significance value for at least one of the plurality of clonotypes. The one or more processors more identify the subject as one of a responder or a non-responder to the first immunotherapy based on the score satisfying a threshold.

In some embodiments, the one or more processors may receive, at a third time prior to an administration of a second immunotherapy to the subject, a third dataset identifying a third plurality of sequence reads. Each sequence read of the third plurality sequence reads may correspond to a respective TRB sequence or TRA sequence derived from TCR encoding polynucleotides present in a third plurality of T cell clones in a third biological sample from the subject. The one or more processors more identify a third distribution of the third plurality of T cell clones belonging to the clonotype in the third biological sample using the third plurality of sequence reads from the third dataset. The one or more processors may receive, at a fourth time subsequent to the administration of the second immunotherapy to the subject, a fourth dataset identifying a fourth plurality of sequence reads. Each sequence read of the fourth plurality sequence reads may correspond to a respective TRB sequence or TRA sequence derived from TCR encoding polynucleotides present in a fourth plurality of T cell clones in a fourth biological sample from the subject.

The one or more processors may, for each clonotype of the plurality of clonotypes in the fourth biological sample: identif ya fourth distribution of the fourth plurality of T cell clones belonging to the clonotype in the second biological sample using the fourth plurality of sequence reads from the dataset; compare the third distribution of the third plurality of T cell clones with the fourth distribution of the fourth plurality of T cells; generate a second significance value indicating an expansion of T cell clones of the clonotype within the fourth biological sample based on comparing the third distribution with the fourth distribution in accordance with a Fisher's exact test; and adjust the third significance value for the clonotype based on a number of clonotypes within a second plurality of clonotypes in the third biological sample and the fourth biological sample, to generate a fourth significance value. The one or more processors may determine a second score identifying a second degree of responsiveness to the second immunotherapy in the subject over the third time and the fourth time based on the fourth significance value for the clonotype. The one or more processors may identify the subject as one of a responder or a non-responder to the second immunotherapy based on the second score satisfying a second threshold. In some embodiments, the first immunotherapy and the second immunotherapy may be distinct types.

In some embodiments, the one or more processors may select an instruction from a plurality of instructions based on identifying the subject as one of the responder or the non-responder to the first immunotherapy or the second immunotherapy. The plurality of instructions may include: a first instruction to continue administering the first immunotherapy responsive to identifying the subject as the responder to the first immunotherapy; a second instruction to discontinue administering the first immunotherapy responsive to identifying the subject as the non-responder to the first immunotherapy; a third instruction to continue administering the second immunotherapy responsive to identifying the subject as the responder to the second immunotherapy; a fourth instruction to discontinue administering the second immunotherapy responsive to identifying the subject as the non-responder to the second immunotherapy; a fifth instruction to continue administering at least one of the first immunotherapy and the second immunotherapy, responsive to identifying the subject as the responder to the first immunotherapy and the second immunotherapy, and a sixth instruction to discontinue administering both the first immunotherapy and the second immunotherapy, responsive to identifying the subject as the non-responder to the first immunotherapy and the second immunotherapy.

In some embodiments, the one or more processors may compare the score to a first threshold and the second score to a second threshold. In some embodiments, the one or more processors may identify the subject as the responder to the first immunotherapy, responsive to the score satisfying the first threshold. In some embodiments, the one or more processors may identify the subject as the non-responder to the first immunotherapy, responsive to the score not satisfying the first threshold. In some embodiments, the one or more processors may idetnify the subject as the responder to the second immunotherapy, responsive to the second score satisfying the second threshold. In some embodiments, the one or more processors may identify the subject as the non-responder to the second immunotherapy, responsive to the second score not satisfying the second threshold.

In some embodiments, the one or more processors may determine the first threshold to compare against the score based on a first frequency of the first plurality of T cells corresponding to each clonotype of the plurality of clonotypes in the first biological sample. In some embodiments, the one or more processors may determine the second threshold to compare against the score based on a second frequency of the third plurality of T cells corresponding to each clonotype of the plurality of clonotypes in the third biological sample. In some embodiments, the one or more processors may generate, for each clonotype of the plurality of clonotypes, the first significance value indicating a two-fold expansion of T cell clones.

In some embodiments, the one or more processors may rescale, for each clonotype of the plurality of clonotypes in the second biological sample, the second distribution of the second plurality of T cell clones of the clonotype based on a first difference between (i) a factor of a first number of the plurality of clonotypes in the second biological sample and (ii) a first number of the second plurality of T cell clones belonging to the clonotype. In some embodiments, the one or more processors may rescale, for each clonotype of the plurality of clonotypes in the fourth biological sample, the second distribution of the second plurality of T cell clones of the clonotype based on a second difference between (i) a factor of a second number of the plurality of clonotypes in the second biological sample and (ii) a second number of the fourth plurality of T cell clones belonging to the clonotype.

In some embodiments, the one or more processors may rescale, for each clonotype of the plurality of clonotypes in the second biological sample, the second distribution of the plurality of T cells of the clonotype to compare against the first distribution of T cells of the clonotype, based on a first difference between (i) a first number of the plurality of clonotypes in the first biological sample and (ii) a second number of the plurality of T cell clones belonging to the clonotype. In some embodiments, the one or more processors may rescale, for each clonotype of the plurality of clonotypes in the fourth biological sample, the fourth distribution of the plurality of T cells of the clonotype to compare against the first distribution of T cells of the clonotype, based on a difference between (i) a third number of the plurality of clonotypes in the third biological sample and (ii) a fourth number of the plurality of T cell clones belonging to the clonotype.

In some embodiments, the one or more processors may adjust the first significance value for each clonotype of the plurality of clonotypes in the second biological sample in accordance with a Bonferroni correction based on a number of the plurality of clonotypes in both the first biological sample and the second biological sample. In some embodiments, the one or more processors may adjust the second significance value for each clonotype of the plurality of clonotypes in the fourth biological sample in accordance with a Bonferroni correction based on a number of the plurality of clonotypes in both the third biological sample and the fourth biological sample.

In some embodiments, the one or more processors may determine that the one or more processors, that the second distribution of the second plurality of T cell clones belonging to at least one clonotype of the plurality of clonotypes does not satisfy a threshold. In some embodiments, the one or more processors may assign the second distribution of the second plurality of T cell clones belonging to the at least one clonotype to a value based on a factor of a number of the plurality of clonotypes in the second biological sample. In some embodiments, the one or more processors may determine that the fourth distribution of the fourth plurality of T cell clones belonging to at least one clonotype of the plurality of clonotypes does not satisfy a threshold. In some embodiments, the one or more processors may assign the fourth distribution of the fourth plurality of T cell clones belonging to the at least one clonotype to a value based on a factor of a number of the plurality of clonotypes in the fourth biological sample.

In some embodiments, the one or more processors may remove, from each of the first plurality of sequence reads, second plurality of sequence reads, the third plurality of sequence reads, and the fourth plurality of sequence reads, a subset of sequence reads determined as non-productive based on an identification of recombined TCR alpha CDR3 nucleotide sequences or TCR beta CDR3 nucleotide sequences from silenced alleles. In some embodiments, the one or more processors may identify, for each clonotype of the plurality of clonotypes, the second distribution of the second plurality of T cell clones corresponding to the clonotype based on a respective permutation of variable (V) gene, a joining (J) gene, and a nucleotide CDR3 sequence. In some embodiments, the one or more processors may identify, for each clonotype of the plurality of clonotypes, the fourth distribution of the fourth plurality of T cell clones corresponding to the clonotype based on a respective permutation of variable (V) gene, a joining (J) gene, and a nucleotide CDR3 sequence.

In some embodiments, the first, second, third, and/or fourth biological sample may be the same type. In some embodiments, the first immunotherapy or the second immunotherapy may be an anti-cancer vaccine, monoclonal antibody-based immunotherapy, or an immune checkpoint inhibitor. Additionally or alternatively, in some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. In some embodiments, the second biological sample may be obtained at least 1 week, at least 2 weeks, at least 3 weeks, 4 at least weeks, or at least 5 weeks after the first immunotherapy has been administered to the subject. In some embodiments, the fourth biological sample may be obtained at least 1 week, at least 2 weeks, at least 3 weeks, 4 at least weeks, or at least 5 weeks after the second immunotherapy has been administered to the subject.

In some embodiments, the second immunotherapy may be administered at least at least 2 weeks, at least 3 weeks, 4 at least weeks, at least 5 weeks, at least 6 weeks, at least 7 weeks, at least 8 weeks, at least 9 weeks, at least 10 weeks, at least 11 weeks, at least 12 weeks, and at least 13 weeks after the first immunotherapy. In some embodiments, the first, second, third, and/or fourth plurality of T cell clones may include one or more of CD4+ helper T cells, CD8+ cytotoxic T cells, CD8+ CD107+ T cells, central memory T cells, stem-cell-like memory T cells (or stem-like memory T cells), effector memory T cells, Natural killer T cells, Mucosal associated invariant T cells, and γδ T cells. In some embodiments, the first, second, third, and/or fourth biological sample may include plasma, serum, whole blood, or PBMCs.

In any and all embodiments of the methods disclosed herein, the monoclonal antibody-based immunotherapy comprises an antibody, antigen binding fragment, or a derivative thereof. In some embodiments, the monoclonal antibody-based immunotherapy targets a tumor antigen. Examples of tumor antigens include, but are not limited to, CD3, GPA33, HER2/neu, GD2, MUC16, MAGE-1, MAGE-3, BAGE, GAGE-1, GAGE-2, MUM⁻¹, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucosaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

In any and all embodiments of the methods disclosed herein, the immune checkpoint inhibitor may include one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, an anti-LAG-3 antibody, an anti-OX40 antibody, an anti-TIGIT antibody, an anti-B7-H3 antibody, an anti-B7-H4 antibody, or an anti-BTLA antibody.

In some embodiments, the immune checkpoint inhibitor may include pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. In some embodiments, the cancer may be selected from among carcinomas, sarcomas, hematopoietic cancers, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In certain embodiments, the pancreatic cancer comprises pancreatic ductal adenocarcinoma.

An aspect of the present disclosure is directed to a method of identifying a subject responsive to an immunotherapy. In one embodiment, the method comprises determining the level of antigen-specific T cells in a sample of the subject, wherein a significantly expanded level of antigen-specific T cells identifies a subject responsive to the immunotherapy; and recommending, prescribing, or administering the immunotherapy to the subject if the subject is identified as responsive to the immunotherapy. In another embodiment, the antigen-specific T cells comprise CD8+ T cells. In a further embodiment, the antigen-specific T cells comprise CD8+ CD107+ T cells. In some embodiments, the method further comprises isolating a sample from the subject prior to the determining. In some embodiments, the sample comprises a body fluid. In other embodiments, the body fluid comprises peripheral blood. In further embodiments, the body fluid comprises blood plasma, blood serum, or whole blood. In yet further embodiments, the sample comprises peripheral blood mononuclear cells (PBMCs). In one embodiment, the immunotherapy comprises a vaccine. In other embodiments, the vaccine comprises an anti-cancer vaccine. In some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. In yet other embodiments, the immunotherapy comprises an antibody, fragment, or derivative thereof. In some embodiments, the antibody comprises a checkpoint blockade inhibitor. In other embodiments, the checkpoint blockade inhibitor comprises an anti-PD-L1 antibody, anti-CTLA-4, anti-PD1, anti-LAG3, anti-TIM-3, anti-GITR, anti-OX40, anti-TIGIT, anti-4-1BB, anti-B7-H3, anti-B7-H4, or anti-BTLA. In embodiments, the subject is afflicted with cancer. In some embodiments, the cancer comprises pancreatic cancer, lung cancer, colon cancer, stomach cancer, esophagus cancer, breast cancer, ovary cancer, prostate cancer, or liver cancer. In further embodiments, the pancreatic cancer comprises pancreatic ductal adenocarcinoma. In some embodiments, the level is determined 1 week, 2 weeks, 3 weeks, 4 weeks, or 5 weeks after administration of an anti-cancer vaccine. In other embodiments, the level is compared to a control sample. In further embodiments, the control sample comprises a sample of the subject obtained prior to administration of the anti-cancer vaccine. In yet other embodiments, the level is determined by TCR Vβ sequencing. In one embodiment, the method further comprises comparing the number of expanded T cell clones in a first population with the number of expanded T cell clones in a second population by calculating statistical significance that the first population has a 2-fold increase in the T cell clones compared to the second population. In another embodiment, the statistical significance is determined using a Fisher exact test. In some embodiments, the Fisher exact test is applied to the following contingency/categorical table:

| | # cells ∈ T | Rescaled # cells ∉ | Rescaled # cells |
|---|---|---|---|
| | cell clone x | T cell clone x | in sample |
| Baseline sample | nₓ | [N/2] − nₓ | [N/2] |
| Comparative | mₓ | M − mₓ | M |
| sample | | | |

wherein the repertoire size of the Baseline sample is halved. In other embodiments, clones having a fold change

$$\frac{{m}_{x}}{M} < 2 \times \frac{{n}_{x}}{N}$$

of <2 are assigned a P value of 1; and wherein the P value is adjusted using a Bonferroni correction.

An aspect of the present disclosure is directed to methods for monitoring an immunotherapy being administered to a subject. In one embodiment, the method comprises determining the level of antigen-specific T cells in a sample of the subject at a first time point; determining the level of antigen-specific T cells in a sample of the subject at a second time point, wherein a significantly expanded level of the second time point relative to the first time point identifies a subject responsive to the immunotherapy; and recommending, prescribing, or administering the immunotherapy to the subject if the subject is identified as responsive to the immunotherapy. In another embodiment, a reduced level of the second time point relative to the first time point identifies a subject not responsive to the immunotherapy. In some embodiments, a booster can be administered to the subject not responsive to the immunotherapy. In other embodiments, the first time point is before or after the subject begins the immunotherapy. In further embodiments, the second time point is after the subject begins the immunotherapy. In yet other embodiments, the antigen-specific T cells are specific to the immunotherapy. In another embodiment, the antigen-specific T cells comprise CD8+ T cells. In a further embodiment, the antigen-specific T cells comprise CD8+ CD107+ T cells. In some embodiments, the method further comprises isolating a sample from the subject prior to the determining. In some embodiments, the sample comprises a body fluid. In other embodiments, the body fluid comprises peripheral blood. In further embodiments, the body fluid comprises blood plasma, blood serum, or whole blood. In yet further embodiments, the sample comprises peripheral blood mononuclear cells (PBMCs). In one embodiment, the immunotherapy comprises a vaccine. In other embodiments, the vaccine comprises an anti-cancer vaccine. In some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. In yet other embodiments, the immunotherapy comprises an antibody, fragment, or derivative thereof. In some embodiments, the antibody comprises a checkpoint blockade inhibitor. In other embodiments, the checkpoint blockade inhibitor comprises an anti-PD-L1 antibody, anti-CTLA-4, anti-PD1, anti-LAG3, anti-TIM-3, anti-GITR, anti-OX40, anti-TIGIT, anti-4-1BB, anti-B7-H3, anti-B7-H4, or anti-BTLA. In embodiments, the subject is afflicted with cancer. In some embodiments, the cancer comprises pancreatic cancer, lung cancer, colon cancer, stomach cancer, esophagus cancer, breast cancer, ovary cancer, prostate cancer, or liver cancer. In further embodiments, the pancreatic cancer comprises pancreatic ductal adenocarcinoma. In some embodiments, the level is determined 1 week, 2 weeks, 3 weeks, 4 weeks, or 5 weeks after administration of an anti-cancer vaccine. In other embodiments, the level is compared to a control sample. In further embodiments, the control sample comprises a sample of the subject obtained prior to administration of the anti-cancer vaccine. In yet other embodiments, the level is determined by TCR Vβ sequencing. In one embodiment, the method further comprises comparing the number of expanded T cell clones in a first population with the number of expanded T cell clones in a second population by calculating statistical significance that the first population has a 2-fold increase in the T cell clones compared to the second population. In another embodiment, the statistical significance is determined using a Fisher exact test. In some embodiments, the Fisher exact test is applied to the following contingency/categorical table:

| | # cells ∈ T | Rescaled # cells ∉ | Rescaled # cells |
|---|---|---|---|
| | cell clone x | T cell clone x | in sample |
| Baseline sample | nₓ | [N/2] − nₓ | [N/2] |
| Comparative | mₓ | M − mₓ | M |
| sample | | | |

wherein the repertoire size of the Baseline sample is halved. In other embodiments, clones having a fold change

$$\frac{{m}_{x}}{M} < 2 \times \frac{{n}_{x}}{N}$$

of <2 are assigned a P value of 1; and wherein the P value is adjusted using a Bonferroni correction.

An aspect of the present disclosure is directed to methods of identifying antigen-specific T cells responsive to an immunotherapy. In one embodiment, the method comprises determining the level of antigen-specific T cells in a sample of the subject, wherein a significantly expanded level identifies a subject responsive to the immunotherapy; and recommending, prescribing, or administering the immunotherapy to the subject if the subject is identified as responsive to the immunotherapy. In another embodiment, the antigen-specific T cells comprise CD8+ T cells. In a further embodiment, the antigen-specific T cells comprise CD8+ CD107+ T cells. In some embodiments, the method further comprises isolating a sample from the subject prior to the determining. In some embodiments, the sample comprises a body fluid. In other embodiments, the body fluid comprises peripheral blood. In further embodiments, the body fluid comprises blood plasma, blood serum, or whole blood. In yet further embodiments, the sample comprises peripheral blood mononuclear cells (PBMCs). In one embodiment, the immunotherapy comprises a vaccine. In other embodiments, the vaccine comprises an anti-cancer vaccine. In some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. In yet other embodiments, the immunotherapy comprises an antibody, fragment, or derivative thereof. In some embodiments, the antibody comprises a checkpoint blockade inhibitor. In other embodiments, the checkpoint blockade inhibitor comprises an anti-PD-L1 antibody, anti-CTLA-4, anti-PD1, anti-LAG3, anti-TIM-3, anti-GITR, anti-OX40, anti-TIGIT, anti-4-1BB, anti-B7-H3, anti-B7-H4, or anti-BTLA. In embodiments, the subject is afflicted with cancer. In some embodiments, the cancer comprises pancreatic cancer, lung cancer, colon cancer, stomach cancer, esophagus cancer, breast cancer, ovary cancer, prostate cancer, or liver cancer. In further embodiments, the pancreatic cancer comprises pancreatic ductal adenocarcinoma. In some embodiments, the level is determined 1 week, 2 weeks, 3 weeks, 4 weeks, or 5 weeks after administration of an anti-cancer vaccine. In other embodiments, the level is compared to a control sample. In further embodiments, the control sample comprises a sample of the subject obtained prior to administration of the anti-cancer vaccine. In yet other embodiments, the level is determined by TCR Vβ sequencing. In one embodiment, the method further comprises comparing the number of expanded T cell clones in a first population with the number of expanded T cell clones in a second population by calculating statistical significance that the first population has a 2-fold increase in the T cell clones compared to the second population. In another embodiment, the statistical significance is determined using a Fisher exact test. In some embodiments, the Fisher exact test is applied to the following contingency/categorical table:

| | # cells ∈ T | Rescaled # cells ∉ | Rescaled # cells |
|---|---|---|---|
| | cell clone x | T cell clone x | in sample |
| Baseline sample | nₓ | [N/2] − nₓ | [N/2] |
| Comparative | mₓ | M − mₓ | M |
| sample | | | |

wherein the repertoire size of the Baseline sample is halved. In other embodiments, clones having a fold change

$$\frac{{m}_{x}}{M} < 2 \times \frac{{n}_{x}}{N}$$

of <2 are assigned a P value of 1; and wherein the P value is adjusted using a Bonferroni correction.

An aspect of the present disclosure is directed to methods of tracking TCR Vβ clones over a designated time period in a subject. In one embodiment, the method comprises determining the level of antigen-specific T cells in a sample of the subject at a first time point followed by assessing the CDR3 nucleotide sequence of T cell clones by their β chain sequence (TRB); determining the level of antigen-specific T cells in a sample of the subject at a second time point followed by assessing the CDR3 nucleotide sequence of T cell clones by their β chain sequence (TRB), wherein a significantly expanded level of the second time point relative to the first time point identifies a subject responsive to the immunotherapy; and recommending, prescribing, or administering the immunotherapy to the subject if the subject is identified as responsive to the immunotherapy. In another embodiment, the antigen-specific T cells comprise CD8+ T cells. In a further embodiment, the antigen-specific T cells comprise CD8+ CD107+ T cells. In some embodiments, the method further comprises isolating a sample from the subject prior to the determining. In some embodiments, the sample comprises a body fluid. In other embodiments, the body fluid comprises peripheral blood. In further embodiments, the body fluid comprises blood plasma, blood serum, or whole blood. In yet further embodiments, the sample comprises peripheral blood mononuclear cells (PBMCs). In one embodiment, the immunotherapy comprises a vaccine. In other embodiments, the vaccine comprises an anti-cancer vaccine. In some embodiments, the anti-cancer vaccine comprises a nucleic acid immunotherapy. In other embodiments, the nucleic acid immunotherapy comprises Individual Neoantigen-Specific Immunotherapy (iNeST). In further embodiments, the iNeST comprises autogene cevumeran. In yet other embodiments, the immunotherapy comprises an antibody, fragment, or derivative thereof. In some embodiments, the antibody comprises a checkpoint blockade inhibitor. In other embodiments, the checkpoint blockade inhibitor comprises an anti-PD-L1 antibody, anti-CTLA-4, anti-PD1, anti-LAG3, anti-TIM-3, anti-GITR, anti-OX40, anti-TIGIT, anti-4-1BB, anti-B7-H3, anti-B7-H4, or anti-BTLA. In embodiments, the subject is afflicted with cancer. In some embodiments, the cancer comprises pancreatic cancer, lung cancer, colon cancer, stomach cancer, esophagus cancer, breast cancer, ovary cancer, prostate cancer, or liver cancer. In further embodiments, the pancreatic cancer comprises pancreatic ductal adenocarcinoma. In some embodiments, the level is determined 1 week, 2 weeks, 3 weeks, 4 weeks, or 5 weeks after administration of an anti-cancer vaccine. In other embodiments, the level is compared to a control sample. In further embodiments, the control sample comprises a sample of the subject obtained prior to administration of the anti-cancer vaccine. In yet other embodiments, the level is determined by TCR Vβ sequencing. In one embodiment, the method further comprises comparing the number of expanded T cell clones in a first population with the number of expanded T cell clones in a second population by calculating statistical significance that the first population has a 2-fold increase in the T cell clones compared to the second population. In another embodiment, the statistical significance is determined using a Fisher exact test. In some embodiments, the Fisher exact test is applied to the following contingency/categorical table:

| | # cells ∈ T | Rescaled # cells ∉ | Rescaled # cells |
|---|---|---|---|
| | cell clone x | T cell clone x | in sample |
| Baseline sample | nₓ | [N/2] − nₓ | [N/2] |
| Comparative | mₓ | M − mₓ | M |
| sample | | | |

wherein the repertoire size of the Baseline sample is halved. In other embodiments, clones

$$\frac{{m}_{x}}{M} < 2 \times \frac{{n}_{x}}{N}$$

having a fold change of <2 are assigned a P value of 1; and wherein the P value is adjusted using a Bonferroni correction.

### BRIEF DESCRIPTION OF THE FIGURES

FIGS. 1A-1D show trial design, safety, and feasibility. (FIG. 1A) Trial design. (FIG. 1B) CONSORT diagram. (FIG. 1C) Percentage of grade 3 adverse events (AEs) attributable to atezolizumab and autogene cevumeran in atezolizumab (n=19) and autogene cevumeran (n=16) safety evaluable patients. Blue line=study-defined safety threshold (25%). (FIG. 1D) Achieved and benchmarked times to atezolizumab (left), and first dose of autogene cevumeran (middle). (Right) Number of autogene cevumeran doses. Red line=median, error bars=95% confidence intervals, dotted lines=zone of clinical indifference. n—individual patients; *=patients treated in protocol-specified sequence. P value by two-tailed unpaired t test.

FIGS. 2A-2D show immune response of autogene cevumeran. (FIG. 2A) Immune responders and non-responders in n=16 biomarker evaluable patients. (FIG. 2B) Cumulative percentage of autogene cevumeran expanded T cells by TCR Vβ clone tracking (left), and IFNγ ELISpot (right) in blood. Colored rectangles indicate protocol-specified treatment sequence. *=altered treatment sequence for patients 3, 4, 14, and 18. Triangles=purification points for single cell sequencing in (FIG. 2D). (FIG. 2C) Proportion of patients with mono- or polytope responses to vaccine neoantigens. (FIG. 2D) Single cell transcriptomes of autogene cevumeran expanded CD8⁺ T cells in 4 immune responders. (Far left) Dots=purified bulk blood CD8⁺ T cells. Colored dots=autogene cevumeran expanded T cell clones as identified by TCR Vβ clone tracking in (FIG. 2B). (Middle, and far right) Transcriptional phenotypes, and lytic granule (perforin 1, and granzyme B) expression by autogene cevumeran expanded and non-expanded CD8⁺ T cells.

FIGS. 3A and 3B show vaccine response and recurrence. Recurrence-free survival (RFS) from resection (FIG. 3A) and from landmark time (upon completion of all 8 autogene cevumeran priming doses); (FIG. 3B) stratified by immune response in n=16 biomarker evaluable patients. n—individual patients; P values by two-tailed log-rank test.

FIGS. 4A and 4B show vaccine response, tumor clonality, and neoantigen quality. (FIG. 4A) Shannon entropy (S) of tumor clones in responders and non-responders. (FIG. 4B) Receiver operating curve indicating the ability of neoantigen quality as a continuous variable to classify neoantigens as inducer or non-inducers of an immune response. Dotted line=all neoantigens included in individualized mRNA vaccines. P values by two-tailed Mann Whitney test.

FIGS. 5A-5C show neoantigen-specific intrahepatic T cells and a micrometastasis. Clinical and immunologic snapshot (FIG. 5A), histology, T cell (FIG. 5B), and oncogene (FIG. 5C) composition of a responder who developed a disappearing post-vaccination intrahepatic lymphoid aggregate.

FIGS. 6A and 6B show patient demographics, clinical characteristics, and toxicity. (FIG. 6A) Demographics and clinical characteristics of evaluable patients (n=19). (FIG. 6B) Frequency of grade 1 and 2 adverse events attributable to atezolizumab (left) and autogene cevumeran (right) in evaluable patients who received each drug. n—individual patients.

FIGS. 7A-7D show assays to detect T cell specificity to autogene cevumeran neoantigens. (FIG. 7A) Schematic of assays to detect T cell specificity to autogene cevumeran neoantigens. (FIG. 7A, left) Assay 1: T cell clonal expansion. To detect if autogene cevumeran expands T cells, TCR Vβ sequencing was used to identify T cell clones that expand in the peripheral blood after autogene cevumeran vaccination. Green line=trajectory of an individual expanded T cell clone. Black line=geometric mean trajectory of all expanded T cell clones. Red line=cumulative percentage of all expanded T cell clones. Black triangles=time of surgery; blue triangle=time of atezolizumab; green triangles=times of autogene cevumeran; yellow bars=mFOLFIRINOX cycles. Dotted black line=detection threshold. P values by two-tailed Fishers exact test. (FIG. 7A, middle) Assay 2: IFNγ ELISpot. To detect if autogene cevumeran expands T cells specific to autogene cevumeran neoantigens, peripheral blood mononuclear cells (PBMCs) were collected post-atezolizumab/preautogene cevumeran vaccination (pre-vaccine), 1-3 weeks post-autogene cevumeran priming doses (postpriming), and 5-6 weeks post-mFOLFIRINOX (post-chemo). PBMCs were then restimulated with autogene cevumeran neopeptides (27 amino acids long), and IFNγ production was measured by ELISpot assays. Controls (4 left to right panels)=IFNγ production following restimulation without neopeptides (media alone), with a 27 peptide pool of HLA class-I restricted pathogen-derived T-cell epitopes (CEF), with a 23-peptide pool of HLA class-II restricted pathogen-derived T-cell epitopes (CEFT), or with anti-CD3 stimulation. P values by distribution free resampling. (FIG. 7A, right) Assay 1 confirmation: In vitro neoantigen-specific activation and TCR cloning. To confirm autogene cevumeran-expanded T cell clones in assay 1 are neoantigen-specific, we stimulate autogene cevumeran expanded PBMCs collected 1-3 weeks post-autogene cevumeran priming doses (post-priming) in vitro with computationally predicted minimal neoepitopes (8-14 amino acids long). CD8+ T cells that either express (CD107a+) or do not express (CD107a−) the degranulation marker CD107a were purified, and identify clones with greater proportion of CD107a+ versus CD107a− cells as in vitro neoepitope-expanded T cell clones. Overlap of in vitro neoepitope-activated T cell clones with in vivo autogene cevumeran-expanded T cell clones from assay 1 were examined. To further confirm neoantigen-specificity, T cell receptors (TCRβ) of select autogene cevumeran-expanded T cell clones from assay 1 were cloned, TCRβ were expressed in donor T cells, TCR transduced donor T cells were cultured in vitro with HLA-matched, neoepitope-pulsed, antigen presenting cells (HLA transduced K562 cells), and examine activation (4-1BB expression) on TCR-transduced T cells. P values by two-tailed Fishers exact test. (FIG. 7B) Representative flow cytometry gating strategies. (FIG. 7C) Representative examples of assay results in n=4 patients (patients 1, 10, 11, and 5). (FIG. 7C, left) Assay 1: T cell clonal expansion in patients 1, 10, 11, and 5. P value by two-tailed Fishers exact test. (FIG. 7C, middle): Assay 2: IFNγ ELISpot in patients 1, 10, 11, and 5. P value by distribution free resampling. (FIG. 7C, right): Assay 1 confirmation: In vitro neoantigen-specific expansion. Flow cytometry plots show CD107a expression on CD8+ T cells with neopeptide or control (DMSO) stimulation. Dot plots show number of cells per T cell clone that are CD107ahigh versus CD107alow. Each circle=individual T cell clone. Green squares=clones also detected in assay 1. Diagonal marks where CD107a+ clone frequency=CD107a− clone frequency. P value by one-tailed binomial test with Bonferroni correction. Venn diagram shows overlap of autogene cevumeran-expanded T cell clones (Assay 1), and in vitro neoantigen-specific (neoantigen-specific) T cell clones. For patient 11, though no overlap of vaccine expanded and in vitro immunodominant neoantigen-specific T cell clones was detected, immunodominant neoantigen-specific T cell clones were shown to have similar in vivo expansion trajectories as vaccine expanded clones in other patients (FIG. 7C, right, bottom) Assay 1 confirmation: TCR cloning. Flow cytometry plots show 4-1BB expression on putative neoantigen-specific TCR-transduced CD8+ T cells cultured with HLA matched, neopeptide pulsed antigen presenting cells. n—number of clones. (FIG. 7D) Autogene cevumeran-expanded T cell clones (n=30 clones in n=8 patients, expansion determined by Assay 1) and fraction identified as neoantigen-specific (determined by Assay 1 confirmation).

FIGS. 8A-8E show autogene cevumeran expands multiple T cell clones specific to vaccinated neoantigens. (FIG. 8A, top rows) T cell clonal expansion in responders and non-responders. Green line=trajectory of an individual expanded T cell clone. Black line=mean trajectory of all expanded T cell clones. Red line=cumulative percentage of all expanded T cell clones. Black triangles=time of surgery; blue triangle=time of atezolizumab; green triangles=times of autogene cevumeran; yellow bars=mFOLFIRINOX cycles 1, 2, 4, 6, 8, 10, and 12. n—clones or individual patients as noted. P values by two-tailed Fishers exact test. (FIGS. 8A-8D, bottom row) T cell IFNγ production by ELISpot in responders and non-responders with individual autogene cevumeran neopeptides (neopeptides) or controls. Controls (4 left to right panels)=IFNγ production following restimulation without neopeptides (media alone), with a 27-peptide pool of HLA class-I restricted pathogen derived T-cell epitopes (CEF), with a 23-peptide pool of HLA class-II restricted pathogen-derived T-cell epitopes (CEFT), or with anti-CD3 stimulation. (FIG. 8E) Median number of autogene cevumeran-expanded T cell clones per patient (left). Maximal percentage of all blood T cells achieved by autogene cevumeran-expanded T cell clones before (pre) and after (post) vaccination (middle left). Number of immunogenic neoantigens per patient (middle right). Autogene cevumeran-induced T cells as a percent of all blood T cells at final available follow-up for each patient (far right). P value by two-tailed paired t-test; n—individual patients.

FIGS. 9A-9E show autogene cevumeran activates neoantigen-specific polyfunctional effector CD8+ T cells. (FIG. 9A) Peripheral blood CD8+ T cell proliferation (Ki67) and activation (PD-1, LAG-3, TIM-3, HLA-DR) pre and serially post atezolizumab, autogene cevumeran, and mFOLFIRINOX in n=7 of 8 responders and n=7 of 8 non-responders with available data. (FIG. 9B) Uniform Manifold Approximation and Projection (UMAP) plots of single peripheral blood T cells in n=4 patients (patients 1, 10, 11, and 29) stratified by lineage (CD8 vs. CD4, left), patient (middle), and autogene cevumeran-expanded clones (right; expanded clones identified in FIGS. 8A-8D). Single cells purified post-autogene cevumeran priming doses at time points indicated in FIG. 2B (arrows). (FIGS. 9C-9D) UMAP plots of single peripheral blood T cells in n=4 patients (patient 1, 10, 11, and 29) stratified by CD8+ T cell naïve, dysfunctional, memory, and effector transcriptional phenotypes (FIG. 9C) and select individual phenotyping defining markers (FIG. 9D).

| Naive | Dysfunctional | Memory | Effector |
|---|---|---|---|
| SELL | TIGIT | EOMES | PRF1 |
| CCR7 | TOX | GZMK | GZMB |
| IL7R | LAG3 | CXCR3 | GNLY |
| BCL2 | ENTPD1 | TCF1 | IFNG |
| PECAM1 | CXCL13 | ID3 | EOMES |
| TCF7 | HAVCR2 | STAT4 | ZEB2 |
| BACH2 | GZMB | CCR7 | E2F7 |
| LEF1 | | SELL | TBX21 |
| | | | PDCD1 |
| | | | CXCR3 |
| | | | FAS |

(FIG. 9E) Flow cytometry plots show polyfunctiona cytokine (IFNγ, TNF-α) production by neopeptide-stimulated CD8+ T cells. Flow cytometry gating strategies are as in FIG. 7B.

FIGS. 10A-10D show autogene cevumeran expanded T cell clones do not overlap with atezolizumab-expanded T cell clones. (FIG. 10A) Cumulative percentage of atezolizumab-induced T cells by TCR Vβ clone tracking. *=altered treatment schedules for patients 3, 4, 14, and 18. (FIGS. 10B-10C) T cell clonal expansion with atezolizumab in autogene cevumeran responders and non-responders. Red box=atezolizumab responder. Blue line=trajectory of an individual atezolizumab expanded T cell clone. Black line=geometric mean trajectory of all atezolizumab expanded T cell clones. Red line=cumulative percentage of all atezolizumab expanded T cell clones. Black triangle=time of surgery; blue triangle=time of atezolizumab; green triangles=times of autogene cevumeran; yellow bars=mFOLFIRINOX cycles 1, 2, 4, 6, 8, 10, and 12. n—clones or individual patients as noted. (FIG. 10D) Venn diagrams show overlap of autogene cevumeran-expanded T cell clones (as identified in FIG. 2A; FIGS. 8A-8E) with atezolizumab-expanded T cell pools in autogene cevumeran responders and non-responders.

FIGS. 11A-11E show overall survival, recurrence-free survival, prognostic, and predictive correlations. (FIG. 11A) Overall survival and recurrence-free survival in all evaluable patients. n—individual patients. (FIG. 11B) Recurrence-free survival in autogene cevumeran-treated biomarker evaluable patients stratified by atezolizumab response. n—individual patients. (FIG. 11C) Number of cycles of mFOLFIRINOX in autogene cevumeran responders and non-responders. n—individual patients. (FIG. 11D) Clinical characteristics of autogene cevumeran responders and non-responders. n—individual patients. (FIG. 11E) Intratumoral T cell infiltration in autogene cevumeran responders and non-responders. n—individual patients.

FIG. 12 shows frequency of immunogenic neoantigens. Pie chart of frequency of immunogenic neoantigens in all patients (left) and autogene cevumeran responders (right) determined by a positive IFNγ ELISpot assay. n—neoantigens or patients as noted.

FIG. 13 shows mutational load and recurrence-free survival. Total number of nonsynonymous mutations in autogene cevumeran responders and non-responders.

FIG. 14 shows digital droplet PCR of TP53R175H in liver lesion and controls. The plots show the number of droplets that contain the TP53 wild type (WT) or the TP53R175H mutation in the intrahepatic liver lesion in patient 29 (FIGS. 5A-5C), and in positive (positive) and negative (gDNA) controls.

FIGS. 15A and 15B show patient treatment characteristics.

FIG. 16 shows individualized mRNA neoantigen vaccines in pancreatic cancer.

Without wishing to be bound by theory, individualized mRNA neoantigen vaccines expand neoantigen-specific T cells and delays recurrence.

FIGS. 17A and 17B show vaccine immunogenicity.

FIG. 18 shows mRNA vaccines expand polyclonal T cells distinct from anti-PDL1.

FIG. 19 shows neoantigen-specificity of vaccine-expanded clones.

FIG. 20 shows vaccine immunogenicity.

FIGS. 21A and 21B show mRNA vaccines induce a substantial, polyclonal, durable T cell response.

FIG. 22 shows mRNA vaccines expand polyfunctional CD8⁺ T cells.

FIG. 23 shows vaccine immunogenicity.

FIGS. 24A-24C show mRNA vaccine response correlates with delayed recurrence.

FIG. 25 depicts a block diagram of a system for evaluating responsiveness to types of immunotherapy in subjects suffering from cancer in accordance with an illustrative embodiment.

FIG. 26A depicts a block diagram of a process for processing gene sequence datasets in the system evaluating responsiveness to types of immunotherapy, in accordance with an illustrative embodiment.

FIG. 26B depicts a block diagram of a process for calculating significance values in the system evaluating responsiveness to types of immunotherapy, in accordance with an illustrative embodiment.

FIG. 26C depicts a block diagram of a process for providing instructions in connection with administration of immunotherapies in the system evaluating responsiveness to types of immunotherapy, in accordance with an illustrative embodiment.

FIG. 27 depicts a flow diagram of a method of determining a likelihood of responsiveness to an immunotherapy in a subject suffering from cancer, in accordance with an illustrative embodiment.

FIGS. 28A-28C depict flow diagrams of a method of monitoring responsiveness to at least one type of immunotherapy in a subject suffering from cancer, in accordance with an illustrative embodiment.

FIG. 29 depicts a block diagram of a server system and a client computer system in accordance with an illustrative embodiment.

### DETAILED DESCRIPTION

It is to be appreciated that certain aspects, modes, embodiments, variations and features of the present methods are described below in various levels of detail in order to provide a substantial understanding of the present technology.

Detailed descriptions of one or more preferred embodiments are provided herein. It is to be understood, however, that the present technology may be embodied in various forms. Therefore, specific details disclosed herein are not to be interpreted as limiting, but rather as a basis for the claims and as a representative basis for teaching one skilled in the art to employ the present technology in any appropriate manner.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

An “antigen” is a molecule or entity to which an antibody or a T cell receptor binds. In some embodiments, an antigen is or comprises a polypeptide or portion thereof. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present technology are provided in a crude form.

Antigens that are expressed in or by tumor cells are referred to as “tumor associated antigens.” A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as “neoepitopes.” Neoepitopes are completely foreign to the body and thus would not produce an immune response against healthy tissue or be masked by the protective components of the immune system.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 104 M⁻¹ greater or at least 105 M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target antigen (e.g., a tumor antigen) will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multi-specific antibodies, bispecific antibodies, etc.,) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CHI, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi-specific antibodies formed from antibody fragments.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein, a “clonotype” refers to a set of adaptive immune cells (e.g., T cells) that are the clonal progeny of a fully recombined, unmutated common ancestor. T cell clonotypes are generally distinguished by the nucleotide sequence of the rearranged TCR, which does not undergo somatic hypermutation (SHM) in the majority of vertebrate species.

“Complementarity-determining regions” or “CDRs” are part of the variable chains in immunoglobulins (antibodies) and T cell receptors, generated by B-cells and T-cells respectively, where these molecules bind to their specific antigen. The main determinants of target recognition by T cell receptors are the complementarity-determining region (CDR) loops. Five of the six TCR CDRs (VαCDR1, VαCDR2, VαCDR3, VβCDR1, and VβCDR2,) adopt a limited number of backbone conformations, known as the “canonical classes”, whereas the remaining Vβ CDR3 of the TCR is structurally diverse.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, “epitope” refers to a portion of an antigen that is recognized by the immune system in the appropriate context, specifically by antibodies, B cells, or T cells. Epitopes may include B cell epitopes (e.g., predicted B cell reactive epitopes) and T cell epitopes (e.g., predicted T cell reactive epitopes). B cell epitopes (e.g., predicted B cell reactive epitopes) refer to a specific region of the antigen that is recognized by an antibody. T-cell epitopes (e.g., predicted T cell reactive epitopes) are peptide sequences which, in association with proteins on APC, are required for recognition by specific T-cells. T cell epitopes (e.g., predicted T cell reactive epitopes) are processed intracellularly and presented on the surface of APCs, where they are bound to MHC molecules including MHC class II and MHC class I molecules. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Conformational epitopes are epitopes that are defined by the conformational structure of the native protein. These epitopes may be continuous or discontinuous (i.e., may be components of the epitope can be situated on disparate parts of the protein, which are brought close to each other in the folded native protein structure).

“Immune checkpoint inhibitor(s)” as used herein refers to molecules that completely or partially reduce, inhibit, interfere with or modulate the activity of one or more checkpoint proteins. Checkpoint proteins regulate T-cell activation or function. Checkpoint proteins include, but are not limited to CTLA-4 and its ligands CD80 and CD86; PD-1 and its ligands PDL1 and PDL2; LAGS, B7-H3, B7-H4, TIM3, ICOS, and BTLA (Pardoll et al. Nature Reviews Cancer 12: 252-264 (2012)).

As used herein, the terms “individual”, “patient”, or “subject” can be an individual organism, a vertebrate, a mammal, or a human. In some embodiments, the individual, patient or subject is a human.

As used herein the term “neoepitope” or “neo antigen” is understood in the art to refer to an epitope that emerges or develops in a subject after exposure to or occurrence of a particular event (e.g., development or progression of a particular disease, disorder or condition, e.g., infection, cancer, stage of cancer, etc). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neoepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope).

As used herein, a “T-cell receptor” or “TCR” refers to an antigen-binding molecule expressed on the surface of T cells, and is a heterodimer consisting of either an α and β chain or a γ and δ chain. The α, and β chains are formed from the somatic rearrangement of the respective V, D, and Jgenes of the TCR loci. The random combination of these genes, alongside further diversification mechanisms (e.g., random nucleotide addition), are estimated to yield trillions of unique TCRβ. TCRα chains are made from the V and J genes, while TCRβ chains are assembled from the V, D, and J genes. In TCRβ, sequence, and structural diversity is concentrated in six hypervariable loops, known as the complementarity determining regions (CDRs). There are three in the TCRα chain (CDRα1-CDRα3) and three in the TCRβ chain (CDRβ1-CDRβ3).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The term “vaccine” as used herein is a preparation used to enhance protective immunity against cancer, or infectious agents such as viruses, fungi, bacteria and other pathogens. A vaccine may be useful as a prophylactic agent or a therapeutic agent. Vaccines contain cells or antigens which, when administered to the body, induce an immune response with the production of antibodies and immune lymphocytes (T-cells and B-cells).

### Anti-Cancer Vaccines

Nucleic acid cancer vaccines (e.g., mRNA vaccines) may encode one or more peptide epitopes (which are portions of personalized cancer antigens). Portions of personalized cancer antigens are segments of personalized cancer antigens that are less than the full-length personalized cancer antigen. In one embodiment, the nucleic acid cancer vaccine is composed of open reading frames that may contain any number of peptide epitopes. In some embodiments the nucleic acid cancer vaccine is composed of open reading frames encoding 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 45 or more, 50 or more, 55 or more, 60 or more, 65 or more, 70 or more, 75 or more, 80 or more, 85 or more, 90 or more, 95 or more, 100 or more, 105 or more, 110 or more, 115 or more, 120 or more, 125 or more, 130 or more, 135 or more, 140 or more, 145 or more, 150 or more, 155 or more, 160 or more, 165 or more, 170 or more, 175 or more, 180 or more, 185 or more, 190 or more, 195 or more, or 200 or more peptide epitopes. In other embodiments the nucleic acid cancer vaccine is composed of open reading frames encoding 200 or less, 195 or less, 190 or less, 185 or less, 180 or less, 175 or less, 170 or less, 165 or less, 160 or less, 155 or less, 150 or less, 145 or less, 140 or less, 135 or less, 130 or less, 125 or less, 120 or less, 115 or less, 110 or less, 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, or 10 or less, or 5 or less peptide epitopes. In other embodiments the nucleic acid cancer vaccine is composed of open reading frames encoding up to 200, up to 195, up to 190, up to 185, up to 180, up to 175, up to 170, up to 165, up to 160, up to 155, up to 150, up to 145, up to 140, up to 135, up to 130, up to 125, up to 120, up to 115, up to 110, up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, up to 10 peptide epitopes, up to 5 peptide epitopes, or up to 4 peptide epitopes. In certain embodiments, the nucleic acid cancer vaccines include open reading frames that encode epitopes or antigens based on specific mutations (neoepitopes) and/or those expressed by cancer-germline genes (antigens common to tumors found in multiple patients).

Each peptide epitope of the vaccines may be any length that is reasonable for an epitope. In certain embodiments, at least two (e.g., at least 3, at least 4, at least 5, at least 6, at least 7, at least 8, at least 9, at least 10, at least 11, at least 12, at least 13, at least 14, at least 15, and up to and including all) of the peptide epitopes in a nucleic acid cancer vaccine are different lengths. In some embodiments, the length of at least one of the peptide epitopes is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100 amino acids. In other embodiments, the length of at least one of the peptide epitopes is 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, 14 or less, 13 or less, 12 or less, 11 or less, 10 or less, 9 or less, 8 or less, 7 or less, 6 or less, 5 or less, 4 or less, 3 or less, or 2 or less amino acids. In other embodiments, the length of at least one of the peptide epitopes is up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, or up to 10 amino acids.

In some embodiments, each of the peptide epitopes encoded by the nucleic acid cancer vaccine may have a different length. In certain embodiments, at least one of the peptide epitopes has a different length than another peptide epitope encoded by the nucleic acid cancer vaccine. Each peptide epitope may be any length that is reasonable for an epitope.

In some embodiments, the at least one peptide epitope recognized by T cell clones is a personalized neoantigen (or neoepitope) specific for a cancer subject. A personalized neoantigen (or neoepitope) is present in a tumor of an individual and is not expressed or expressed at low levels in normal non-cancerous tissue of the individual. The personalized neoantigen may or may not be present in tumors of other individuals.

In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

In some embodiments the nucleic acid cancer vaccines described herein may include peptide epitopes or antigens based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

In some embodiments, the neoepitopes are 13 residues or less in length and may consist of between about 8 and about 11 residues, particularly 9 or 10 residues. In other embodiments the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

Neoepitopes having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Be, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the alpha-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In some embodiments, the at least one peptide epitope recognized by T cell clones is derived from one or more tumor antigens selected from among MAGE, BAGE, GAGE, NY-ESO-1, Tyrosinase, Melan-A, gp100, CEA, MART-1, HER2, WT1, MUC1, ppCT, Beta-catenin, CDK4, LPGAT1, CASP-8, CDKN2A, HLA-A11d, CLPP, GPNMB, RBAF600, SIRT2, SNRPD1, SNRP116, MART2, MUM-If, MUM-2, MUM-3, Myosin class I, N-ras, OS-9, Elongation factor 2, NFYC, Alpha-actinin-4, Malic enzyme, HLA-A2, Hsp70-2, SETDB1, METTL17, ALDH1A1, CDKN2A, TKT, SEC24A, EXOC8, MRPS5, PABPC1, KIF2C, POLA2, CCT6A, TRRAP, DNMT1, PABPC3, MAGE-A10, FMN2, TMEM48, AKAP13, OR8B3, WASL, MAGEA6, PDS5A, MED13, FLNA, KIB1B, KFIIBP, NARFL, PPFIA4, CDC37L1, MLL3, FLNA, DOPEY2, TTBK2, KIF26B, SPOP, RETSAT, CLINTI, COX7A2, FAM3C, CSMD1, PPPIR3B, CDK12, CSNKIA1, GAS7, MATN, HAUS3, MTFR2, CHTF18, MYADM, HERC1, HSDL1, COA-1, ARTC1, CDC27, FN1, LDLR-FUT fusion protein, neo-PAP, PTPRK and Triosephosphate isomerase.

One important aspect of a neoepitope included in a vaccine is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset. A personalized coding genome may be used as a reference for comparison of neoantigen candidates to determine lack of self-reactivity. In some embodiments, a personalized coding genome is generated from an individualized transcriptome and/or exome.

### Computational Systems and Methods of the Present Technology

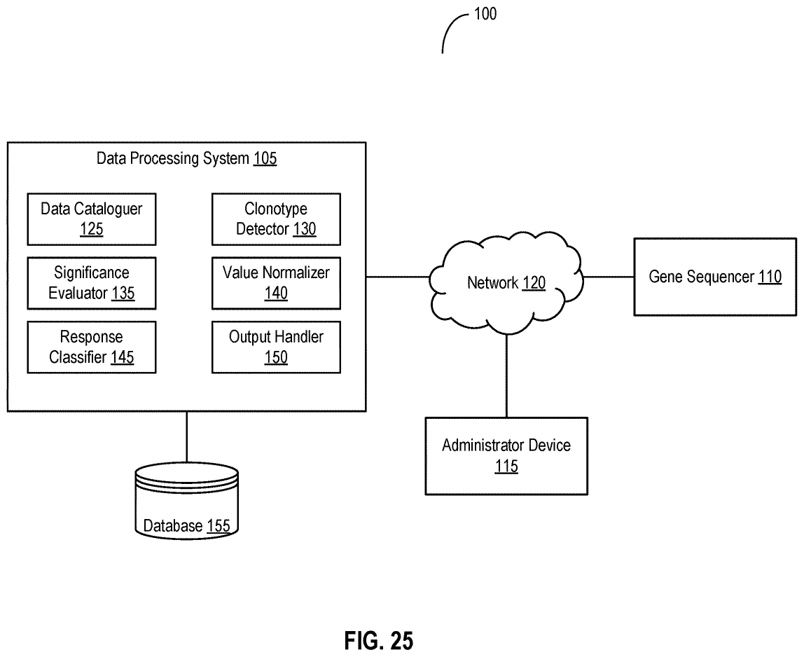

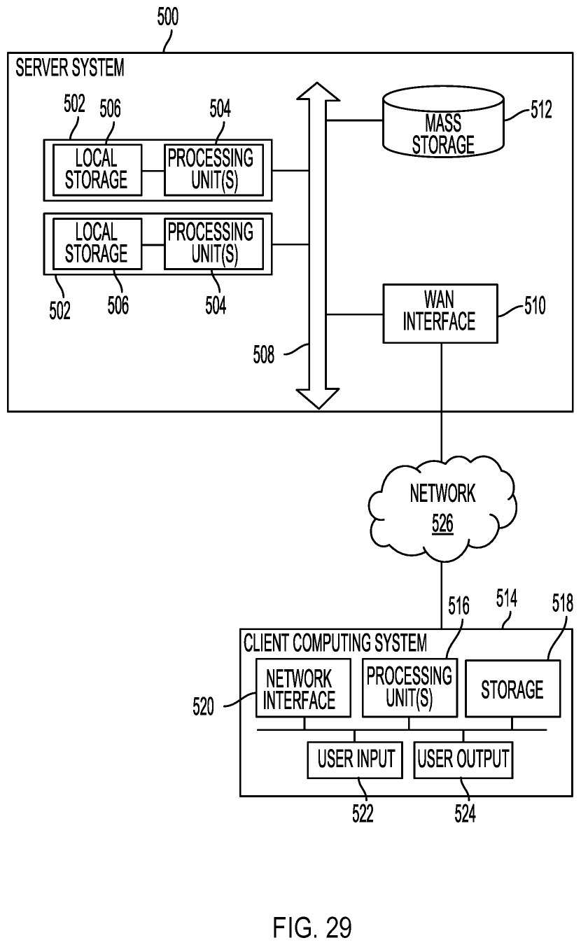

Referring now to FIG. 25, depicted is a block diagram of a system 100 for evaluating responsiveness to types of immunotherapy in subjects suffering from cancer. In brief overview, the system 100 may include at least one data processing system 105, at least one gene sequencer 110, and at least one administrator device 115, communicatively coupled with one another via at least one network 120. The data processing system 105 may include at least one data cataloguer 125, at least one clonotype detector 130, at least one significance evaluator 135, at least one value normalizer 140, at least one response classifier 145, at least one output handler 150, and at least one database 155, among others. Each of the components in the system 100 as detailed herein may be implemented using hardware (e.g., one or more processors coupled with memory), or a combination of hardware and software as detailed herein in conjunction with FIG. 29. Each of the components in the system 100 may implement or execute the functionalities detailed herein, such as those in the Examples.

In further detail, the data processing system 105 can be any computing device comprising one or more processors coupled with memory and software and capable of performing the various processes and tasks described herein. The data processing system 105 may be in communication with the gene sequencer 110 and the administrator device 115, and other devices, via the network 130. The data processing system 105 may be situated, located, or otherwise associated with at least one server group. The server group may correspond to a data center, a branch office, or a site at which one or more servers corresponding to the image processing system 105 is situated.

The gene sequencer 110 can be any computing device comprising one or more processors coupled with memory and software and capable of performing the various processes and tasks described herein. The gene sequencer 110 may be in communication with the data processing system 105 and the administrator device 115, and other devices, via the network 130. The gene sequencer 110 may carry out, execute, or otherwise perform genetic sequencing on biological samples taken from subjects to generate gene sequencing data. The biological samples and the gene sequencing may be performed in connection with administration of an immunotherapy to the subject, such as prior to, middle of, or subsequent to the administration of the immunotherapy. The genetic sequencing carried out by the gene sequencer 110 may be a high throughput, massively parallel sequencing technique (sometimes herein referred to as next generation sequencing), such as pyrosequencing, Reversible dye-terminator sequencing, SOLiD sequencing, Ion semiconductor sequencing, Helioscope single molecule sequencing, among others.

The administrator device 115 can be any computing device comprising one or more processors coupled with memory and software and capable of performing the various processes and tasks described herein. The administrator device 115 may be in communication with the data processing system 105 and gene sequencer 115, and other devices, via the network 130. The administrator device 115 may be associated with an entity (e.g., clinician, doctor, nurse, or hospital staff) managing, handling, or otherwise performing the administration of the immunotherapy on the subject. The administrator device 115 may be situated, located, or otherwise associated with at least one server group. In some embodiments, the administrator device 115 may be part of the data processing system 105. In some embodiments, the administrator device 115 may be separate from the data processing system 105 (e.g., as depicted).

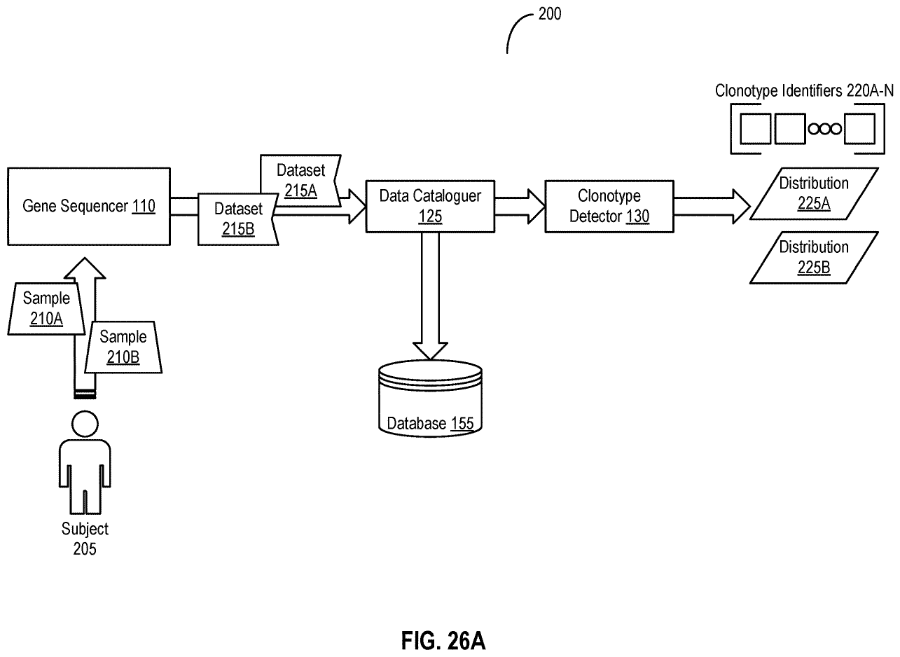

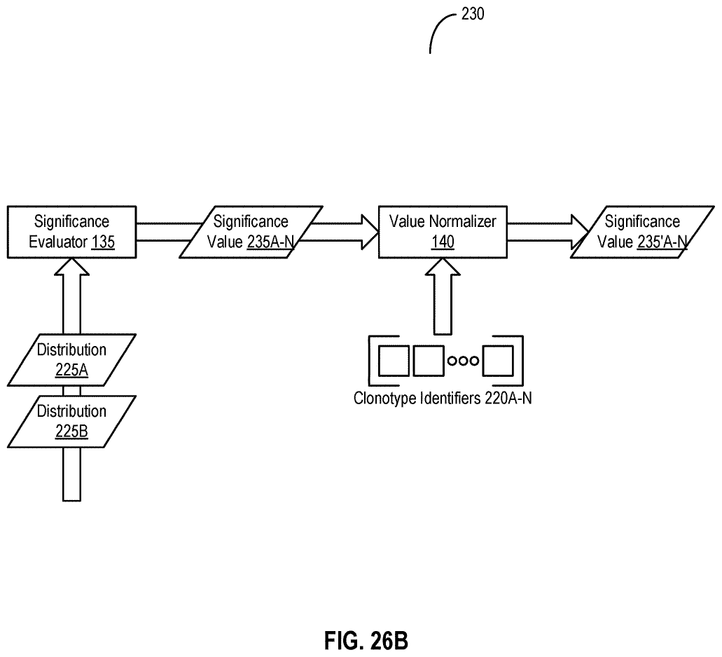

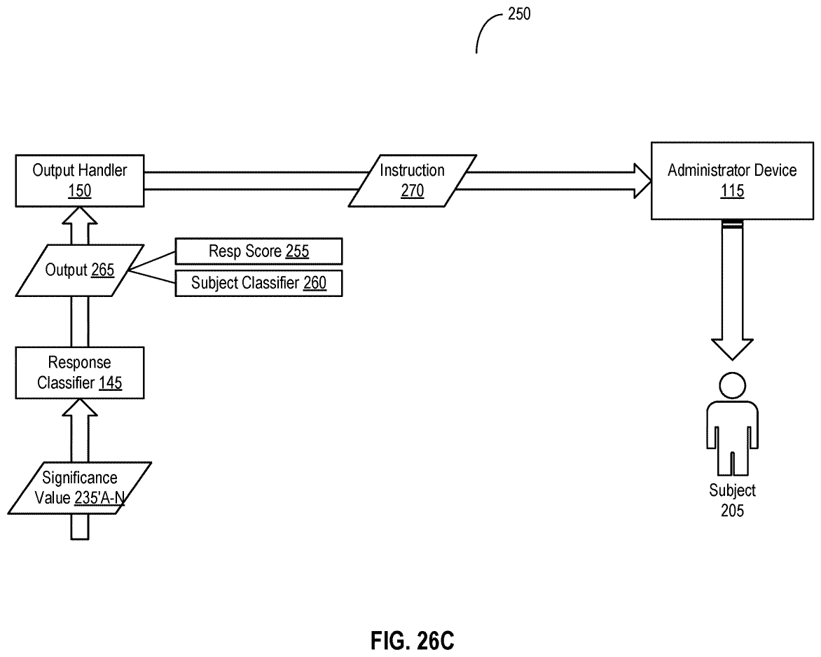

Referring now to FIG. 26A, depicted a block diagram of a process 200 for processing gene sequence datasets in the system evaluating responsiveness to types of immunotherapy. The process 200 may include or correspond to operations performed in the system 100 to acquire and process datasets of sequence reads, in connection with administration of an immunotherapy on a subject 205. The subject 205 may be diagnosed with or suffering from cancer. The cancer affecting the subject 205 may include, for example: carcinomas, sarcomas, hematopoietic cancers. adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

The immunotherapy to administer to the subject 205 may include, for example: anti-cancer vaccine, monoclonal antibody-based immunotherapy, or an immune checkpoint inhibitor, among others. In some embodiments, the anti-cancer vaccine comprises a nucleic acid immunotherapy. The nucleic acid immunotherapy may comprise Individual Neoantigen-Specific Immunotherapy (iNeST). For example, the iNeST may comprise the autogene cevumeran.

In any and all embodiments of the methods disclosed herein, the monoclonal antibody-based immunotherapy comprises an antibody, antigen binding fragment, or a derivative thereof. In some embodiments, the monoclonal antibody-based immunotherapy targets a tumor antigen. Examples of tumor antigens include, but are not limited to, CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

The immune checkpoint inhibitor may include one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, an anti-LAG-3 antibody, an anti-OX40 antibody, an anti-TIGIT antibody, an anti-B7-H3 antibody, an anti-B7-H4 antibody, an anti-BTLA antibody among others. In some embodiments, the immune checkpoint inhibitor may include pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189, among others.

Under the process 200, the gene sequencer 110 may carry out, execute, or otherwise perform gene sequencing on samples taken from at least one subject 205 in connection with an administration of an immunotherapy. The samples may be obtained or taken from a primary site or a metastatic site associated with the cancer in the subject 205. The primary site may correspond to an anatomical location (e.g., an organ, tissue, or other part) in the subject 205 from which the cancer originated. The metastatic site may correspond to another anatomical location to which the cancer spread within the subject 205.

The gene sequencer 110 may perform gene sequencing on at least one first sample 210A (sometimes herein referred to as a reference biological sample) obtained from the subject 205, at a time prior to an administration of an immunotherapy to the subject 205. In other embodiments, the reference biological sample may be obtained from a cancer patient that does not receive the immunotherapy. From performing the gene sequencing, the gene sequencer 110 may output, produce, or otherwise generate at least one first dataset 215A. The first dataset 215A may include or identify a plurality of sequence reads. Each sequence read of the plurality sequence reads may correspond to a respective T-cell receptor beta locus (TRB) sequence or T-cell receptor alpha locus (TRA) sequence derived from T-cell receptor (TCR) encoding polynucleotides present in a plurality of T cell clones in the first sample 210A from the subject 205. With the generation, the gene sequencer 110 may provide, transmit, or otherwise send the first dataset 215A to the data processing system 105. The first dataset 215A may include an indication identifying that the plurality of sequence reads is derived from the first sample 210A at a time prior to the administration of the immunotherapy.

In addition, the gene sequencer 110 may perform gene sequencing on at least one second sample 210B (sometimes referred herein as the comparative sample) obtained from the subject 205, at a time subsequent to the administration of the immunotherapy to the subject 205. The immunotherapy may be of a particular type (e.g., anti-cancer vaccine, monoclonal antibody-based immunotherapy, immune checkpoint inhibitor etc.). The second sample 210B may be taken or obtained from the subject 205 at least 1 week, at least 2 weeks, at least 3 weeks, 4 at least weeks, at least 5 weeks, at least 6 weeks, at least 7 weeks, at least 8 weeks, at least 9 weeks, at least 10 weeks, at least 11 weeks, at least 12 weeks, or at least 13 weeks after the immunotherapy has been administered to the subject 205. The second sample 210B may of the same type of biological sample as the first sample 210A. For example, both the first sample 210A and the second sample 210B may be plasma, serum, whole blood, or peripheral blood mononuclear cells (PBMCs), among others.

From performing the gene sequencing, the gene sequencer 110 may output, produce, or otherwise generate at least one second dataset 215B. The second dataset 215A may include or identify a plurality of sequence reads. Each sequence read of the plurality sequence reads may correspond to a TRB sequence or TRA sequence derived from TCR encoding polynucleotides present in a second plurality of T cell clones the second sample 210B from the subject 205. With the generation, the gene sequencer 110 may transmit, provide, or otherwise send the second dataset 215B to the data processing system 105. The second dataset 215B may include an indication identifying that the plurality of sequence reads is derived form the second sample time 210B at a time subsequent to the administration of the immunotherapy.

The data cataloguer 125 on the data processing system 105 may retrieve, identify, or otherwise receive the first dataset 215A from the gene sequencer 110. Upon receipt, the data cataloguer 125 may store and maintain the first dataset 215A on the database 155. In some embodiments, the data cataloguer 125 may identify the first dataset 215A as derived from the first sample 210A obtained at a time prior to the administration of the immunotherapy based on the indication. The data cataloguer 125 may store and maintain an association between the first dataset 215A and the identification that the first sample 210A was obtained at the time prior to the administration of the immunotherapy, using one or more data structures on the database 155. The data structures may include, for example, a linked list, an array, a table, a matrix, a binary tree, a heap, a stack, or a queue, among others.

In addition, the data cataloguer 125 may retrieve, identify, or otherwise receive the second dataset 215B from the gene sequencer 110. Upon receipt, the data cataloguer 125 may store and maintain the second dataset 215B on the database 155. In some embodiments, the data cataloguer 125 may identify the second dataset 215B as derived from the second sample 210B obtained at a time (e.g., at least 2 weeks) subsequent to the administration of the immunotherapy based on the indication. The data cataloguer 125 may store and maintain an association between the second dataset 215B and the identification that the second sample 210B was obtained at the time prior to the administration of the immunotherapy, using one or more data structures on the database 155. The data structures may include, for example, a linked list, an array, a table, a matrix, a binary tree, a heap, a stack, or a queue, among others.

In some embodiments, the data cataloguer 125 may filter out, delete, or otherwise remove a subset of sequence reads from the plurality of sequence reads in the first dataset 215A and from the plurality of sequence reads in the second dataset 215B. For each sequence read in the first dataset 215A or the second dataset 215B, the data cataloguer 125 may determine whether the sequence read is non-productive based on an identification of recombined TCR alpha CDR3 nucleotide sequences or TCR beta CDR3 nucleotide sequences from silenced alleles. If the sequence read is determined to be non-productive, the data cataloguer 125 may remove the sequence read from the plurality of sequence reads in the first dataset 215A or the second dataset 215B. On the other hand, if the sequence read is not determined to be non-productive, the data cataloguer 125 may maintain the sequence read from the plurality of sequence reads in the first dataset 215A or the second dataset 215B. The data cataloguer 125 may store and maintain the first dataset 215A or the second dataset 215B, with the removal of the subset of sequence reads for additional processing.

The clonotype detector 130 on the data processing system 105 may determine, detect, or otherwise identify the set of clonotypes among the T cell clones in the first sample 210A using the first dataset 215A and the second sample 210B using the second dataset 215B. The T-cell clones in the first sample 210A or the second sample 210B may include one or more of: CD4+ helper T cells, CD8+ cytotoxic T cells, CD8+ CD107+ T cells, central memory T cells, stem-cell-like memory T cells (or stem-like memory T cells), effector memory T cells, Natural killer T cells, Mucosal associated invariant T cells, and TS T cells, among others. The T-cell clones may be associated with or may belong to a set of different clonotypes. Each clonotype may correspond to a respective permutation of variable (V) gene, a joining (J) gene, and a nucleotide CDR3 sequence. In some embodiments, the clonotype detector 130 may use a predefined set of clonotypes to which to search for in the first sample 210A or the second sample 210B.

From the first dataset 215A, the clonotype detector 130 may detect a first set of clonotypes in the first sample 210A. From the second dataset 215B, the clonotype detector 130 may detect a second set of clonotypes in the second sample 210B. The set of clonotypes detected in the first sample 210A and the second sample 210B may at least partially overlap. Upon detecting each clonotype, the clonotype generator 130 may produce, create, or otherwise generate a corresponding clonotype identifier 220A-N (hereinafter generally referred to as clonotypes 220) to include in a set of clonotype identifiers 220. Each clonotype identifier 220 may reference or correspond to a respective clonotype detected in the first sample 210A or the second sample 210B.

For each clonotype detected in the first sample 210A, the clonotype detector 130 may calculate, determine, or otherwise identify a first distribution 225A (sometimes herein referred to as a reference distribution) of the plurality of T cell clones belonging to the clonotype in the first sample 210A using the plurality of sequence reads from the first dataset 210A. The first distribution 225A may define or identify a number or a frequency of the corresponding clonotype among the set of clonotypes in the first sample 210A. The first distribution 225A of the clonotype may function or serve as a reference to which to compare subsequent distribution of the clonotype to determine whether a significance (or effect) of the administration of the immunotherapy on the subject 205. In some embodiments, the clonotype detector 130 may identify the first distribution 225A of the T cell clones belonging to the clonotype based on a respective permutation of variable (V) gene, a joining (J) gene, and a nucleotide CDR3 sequence within the sequence read in the first dataset 210A.

In some embodiments, the clonotype detector 130 may compare the first distribution 225A of the plurality of T cell clones belonging to the clonotype to a threshold value. The threshold value may delineate, specify, or otherwise define a value for the first distribution 225A at which to assign a new value for the first distribution 225A, and may correspond to a lack or minimal observation of the clonotype within the first sample 210A. If the first distribution 225A does not satisfy (e.g., is less than) the threshold, the clonotype detector 130 may set or assign a value to the first distribution 225A. The value may be based on a factor (e.g., 0.25, 0.33, or 0.5) of a number of the plurality of clonotypes in the first sample 210A. The clonotype detector 130 may determine that the clonotype is missing or has a low presence in the first sample 210A. Otherwise, if the first distribution 225A satisfies (e.g., is greater than or equal to) the threshold, the clonotype detector 130 may maintain the value of the first distribution 225A.

Likewise, for each clonotype detected in the second sample 210B, the clonotype detector 130 may calculate, determine, or otherwise identify a second distribution 225B of the plurality of T cell clones belonging to the clonotype in the second sample 210B using the plurality of sequence reads from the second dataset 210B. The second distribution 225B may define or identify a number or a frequency of the corresponding clonotype among the set of clonotypes in the second sample 210B. In some embodiments, the clonotype detector 130 may identify the second distribution 225B of the T cell clones belonging to the clonotype based on a respective permutation of variable (V) gene, a joining (J) gene, and a nucleotide CDR3 sequence within the sequence read in the first dataset 210A.

In some embodiments, the clonotype detector 130 may compare the second distribution 225B of the plurality of T cell clones belonging to the clonotype to a threshold value. The threshold value may delineate, specify, or otherwise define a value for the second distribution 225B at which to assign a new value for the second distribution 225B, and may correspond to a lack or minimal observation of the clonotype within the second sample 210B. If the second distribution 225B does not satisfy (e.g., is less than) the threshold, the clonotype detector 130 may set or assign a value to the second distribution 225B. The value may be based on a factor (e.g., 0.25, 0.33, or 0.5) of a number of the plurality of clonotypes in the second sample 210B. The clonotype detector 130 may identify or determine that the clonotype is missing or has a low presence in the second sample 210B. Otherwise, if the second distribution 225B satisfies (e.g., is greater than or equal to) the threshold, the clonotype detector 130 may maintain the value of the second distribution 225B.

The process 200 may be repeated any number of times in connection with administration of various types of immunotherapies to the subject 205. For example, the subject 205 may be administered with a first immunotherapy of a first type, and then subsequently administered with a second immunotherapy of a second type. The first type of immunotherapy may be different or distinct from the second type of immunotherapy. For example, the first immunotherapy may be a neoantigen vaccine and the second immunotherapy may be an immune checkpoint inhibitor. The second immunotherapy may be administered to the subject 205 at least at least 2 weeks, at least 3 weeks, 4 at least weeks, at least 5 weeks, at least 6 weeks, at least 7 weeks, at least 8 weeks, at least 9 weeks, at least 10 weeks, at least 11 weeks, at least 12 weeks, and at least 13 weeks subsequent to the administration of the first immunotherapy.

Likewise with the second immunotherapy, the gene sequencer 110 may generate the first dataset 215A using the first sample 210A obtained at a time prior to the administration of the second immunotherapy. The gene sequencer 110 may generate the second dataset 215B using the second sample 210B obtained at a time subsequent to the administration of the second immunotherapy. The second sample 210B may be obtained at least 1 week, at least 2 weeks, at least 3 weeks, 4 at least weeks, or at least 5 weeks after the second immunotherapy has been administered to the subject 205. The data cataloguer 125 may receive the first dataset 215A and the second dataset 215B. The clonotype detector 130 may also detect the set of clonotypes within the first sample 210A and the second sample 210B in connection with the second immunotherapy. For each clonotype, the clonotype detector 130 may identify the first distribution 225A of clonotype within the first sample 210A and the second distribution 225B of clonotype within the second sample 210B.

Referring now to FIG. 26B, depicted is a block diagram of a process 230 for calculating significance values in the system 100 evaluating responsiveness to types of immunotherapy. The process 230 may correspond to or include operations performed in the system 100 to determine effect of the administration of the immunotherapy to the subject 205. Under the process 230, the significance evaluator 135 executing on the data processing system 105 may compare the first distribution 225A of T cell clones belonging to the clonotype with the second distribution 225B of T cell clones belonging to the clonotype. The significance evaluator 135 may perform the comparison for each clonotype of the set of clonotypes in the first sample 210A or the second sample 210B, or a predefined set. The comparison may be in accordance with a statistical significance test, such as a Fisher's exact test, a student's t-test, analysis of variance (ANOVA), chi-squared test, or Spearman's rank coefficient test, among others. In some embodiments, the significance evaluator 135 may perform the Fisher's exact test may be for a N-fold (e.g., two-fold) increase in the T-cell clone of the clonotype in the set of T cell clones.

In some embodiments, the significance evaluator 135 may adjust, modify, or otherwise rescale the first distribution 225A for each clonotype of the set of clonotypes in the first sample 210A in performing the comparison. The rescaling may be performed to evaluate for the N-fold (e.g., two-fold) expansion or increase in the T-cell clone of the clonotype in the set of T cell clones. To rescale, the significance evaluator 135 may identify a number of different clonotypes in first sample 210A and a number of T cell clones belonging to the clonotype. The number of different clonotypes in first sample 210A may be modified by a multiplicative factor (e.g., 0.25, 0.33, or 0.5). In some embodiments, the significance evaluator 135 may calculate or determine a number of cells in the first sample 210A. With the identification, the significance evaluator 135 may calculate or determine a difference between the number of different clonotypes in the first sample 210A and the number of T cell clones belonging to the clonotype. In some embodiments, the significance evaluator 135 may calculate or determine a difference between the number of different clonotypes in the first sample 210A and the number of cells in the first sample 210A. The significance evaluator 135 may use the difference as the rescaled first distribution 225A for each clonotype of the set of clonotypes in the first sample 210A. The rescaled first distribution 225A may be used to compare against the second distribution 225B of T cell clones of the same clonotype.

In some embodiments, the significance evaluator 135 may adjust, modify, or otherwise rescale the second distribution 225B for each clonotype of the set of clonotypes in the second sample 210B in performing the comparison. The rescaling may be performed to evaluate for the N-fold (e.g., two-fold) expansion or increase in the T-cell clone of the clonotype in the set of T cell clones. To rescale, the significance evaluator 135 may identify a number of different clonotypes in the second sample 210B and a number of T cell clones belonging to the clonotype. The number of different clonotypes in second sample 210B may be modified by a multiplicative factor (e.g., 0.25, 0.33, or 0.5). In some embodiments, the significance evaluator 135 may calculate or determine a number of cells in the second sample 210B. With the identification, the significance evaluator 135 may calculate or determine a difference between the number of different clonotypes in second sample 210B and the number of T cell clones belonging to the clonotype. The rescaled second distribution 225B may be used to compare against the rescaled distribution 225A of T cell clones of the same clonotype.

Based on the comparison, the significance evaluator 135 may calculate, determine, or otherwise generate at least one significance value 235A-N (hereinafter generally referred to the significance value 235) for each clonotype in the set of clonotypes. The significance value 235 may identify or indicate an increase or expansion of T cell clones of the clonotype in the second sample 210B. The significance value 235 may be, for example, a numerical value measuring a statistical significance (e.g., a p-value) for the expansion of T cell clones of the clonotype. By extension, the significance value 235 may correspond to a degree of effect of the administration of the immunotherapy on the subject 205. In some embodiments, the significance evaluator 135 may determine the significance value 235 based on the comparison between the distributions 225A and 225B in accordance with the statistical significance test (e.g., Fisher's exact test). In some embodiments, the significance evaluator 135 may determine the significance value 235 based on a comparison of the rescaled distributions 225A and 225B. In some embodiments, the significance value 235 may be determined to indicate a N-fold (e.g., a two-fold) expansion of T cell clones for the clonotype. The determination of the significance value 235 can be repeated across the different clonotypes to generate a set of significance values 235.

The value normalizer 140 executing on the data processing system 105 may modify, set, or otherwise adjust the significance value 235 for each clonotype to output, produce, or otherwise generate at least one significance value 235′A-N (hereinafter generally referred to the significance value 235′). The adjustment of the significance value 235 may be based on a number of clonotypes in the first sample 210A or the second sample 210B. The value normalizer 140 may determine or identify the number of different clonotypes across both the first sample 210A and the second sample 210B. In performing the adjustment, the value normalizer 140 may identify the number of clonotypes based on different number of clonotype identifiers 220 detected across both the first sample 210A and the second sample 210B using the first dataset 215A and the second dataset 215B. In some embodiments, the value normalizer 140 may adjust the significance value 235 in accordance with a statistical control function using the number of different clonotypes across the first sample 210A and the second sample 210B. The statistical correction function may include, for example: Bonferroni correction, Sidak correction, or Holm-Bonferroni correction, among others. The adjustment of the significance value 235 can be repeated across the different clonotypes to generate a set of adjusted significance values 235′.

The process 230 may be repeated any number of times in connection with administration of various types of immunotherapies to the subject 205. For example, the first distribution 225A and the second distribution 225B may be identified for the administration of the second immunotherapy of the second type to the subject 205 as discussed above. The significance value 135 may generate the significance value 235 for each clonotype of the set of clonotypes detected across the first sample 210A and the second sample 210B from the subject 205, in a similar manner as above. The significance value 235 may indicate an expansion of T cell clones of the clonotype as a result of the second immunotherapy. The value normalizer 140 may adjust the significance value 235 for each clonotype to output a corresponding adjusted significance value 235′, again as a similar manner as described above.

Referring now to FIG. 26C, depicted is a block diagram of a process 250 for providing instructions in connection with administration of immunotherapies in the system 100 evaluating responsiveness to types of immunotherapy. The process 250 may correspond to or include operations performed in the system 100 to classify the subject 205 and to provide instructions regarding the administration of the immunotherapy. Under the process 250, the response classifier 145 executing on the data processing system 105 may calculate, generate, or otherwise determine at least one responsiveness score 255 based on the at least one of the significance values 235 for at least one of the set of clonotypes. The responsiveness score 255 may be a numerical value identifying or indicating a degree of responsiveness by the subject 205 to the administered immunotherapy over the time at which the first sample 210A was obtained and the time at which the second sample 210B was obtained. In some embodiments, the response classifier 145 may determine the responsiveness score 255 based on the set of significance values 235 for the set of clonotypes.

Based on the responsiveness score 255, the response classifier 145 may classify, determine, or otherwise identify the subject 205 as one of a responder or a non-responder to the immunotherapy. The subject 205 may be classified as a responder when the significance value 235 and by extension the responsiveness score 255 indicates a substantial (e.g., more than or equal to two-fold) expansion in the T cell clones in response to the administration of the immunotherapy. Conversely, the subject 205 may be classified as a non-responder when the significance value 235 and by extension the responsiveness score 255 indicates a non-substantial (e.g., less than two-fold) expansion in the T cell clones in response to the administration of the immunotherapy.

To identify, the response classifier 145 may compare the responsiveness score 255 to a threshold. The threshold may delineate, specify, or otherwise define a value for the responsiveness score 255 at which to identify the subject 205 as the responder or the non-responder. In some embodiments, the threshold may be a fixed value (e.g., a p-value for statistical significance) against which to compare the responsiveness score 255. In some embodiments, the response classifier 145 may calculate, generate, or otherwise determine the threshold. The determination of the threshold may be based on a frequency of the T cell clones for each clonotype in the first sample 210A. For example, the response classifier 145 may determine the threshold as a factor (e.g., N for an N-fold expansion) of the frequency of the T cell clones for a given clonotype in the first sample 210A.

Based on the comparison, the response classifier 145 may determine whether the responsiveness score 255 satisfies the threshold. If the responsiveness score 255 does not satisfy (e.g., is less than) the threshold, the response classifier 145 may determine or identify the subject 205 as a non-responder to the immunotherapy. On the other hand, if the responsive score 255 satisfies (e.g., is greater than or equal to) the threshold, the response classifier 145 may determine or identify the subject 205 as a responder to the immunotherapy. With the identification, the response classifier 145 may create, produce, or otherwise generate at least one subject classifier 260 indicating the subject 205 as the responder or the non-responder to the immunotherapy. The subject classifier 260 may also identify the immunotherapy that was administered to the subject 205. The response classifier 145 may write, create, or otherwise generate at least one output 265. The output 265 may identify or include the responsiveness score 255 and the subject classifier 260 for the subject 205 in connection with the administration of the immunotherapy.

The output handler 150 executing on the data processing system 105 may create, produce, or otherwise generate at least one instruction 170 based on the identification of the 205 as one of the responder or the non-responder to the immunotherapy. The instruction 170 may be to continue or discontinue the administration of the immunotherapy to the subject 205 based on the subject classifier 260. The instruction 170 may be, for example, a message or notification (e.g., in short message service (SMS) format, Hypertext Markup Language (HTML) format, or Extensible Markup Language (XML) format) with an indication of continuation or discontinuation of the administration of the immunotherapy. When the subject 205 is identified as the responder, the output handler 150 may generate the output 170 to continue the administration of the immunotherapy to the subject 205. When the subject 205 is identified as the non-responder, the output handler 150 may generate the output 170 to discontinue the administration of the immunotherapy to the subject 205.

In some embodiments, the output handler 150 may determine, identify, or otherwise select the instruction 170 to provide from a set of instructions 170 based on the identification of the subject 205 as the responder or the non-responder. The set of instructions 170 may include, for example: a first instruction to continue the administration of the immunotherapy to the subject 205, when the subject 205 is identified as the responder; and a second instruction to discontinue the administration of the immunotherapy to the subject 205, when the subject 205 is identified as the non-responder. With the generation or selection, the output handler 150 may send, transmit, or otherwise provide the instruction 170 to the administrator device 115. The instruction 170 may also include or identify the type of immunotherapy that was administered to the subject 205, the responsiveness score 255, and the subject classifier 260, among others.

The administrator device 115 may retrieve, identify, or otherwise receive the instruction 170 from the data processing system 105. Upon receipt, the administrator device 115 (or an application running on the administrator device 115) may display, render, or otherwise present the information identified in the instruction 170. For example, when the subject 205 is identified as the responder to the immunotherapy, the administrator device 115 may display the instruction 170 to continue the administration of the immunotherapy. A user of the administrator device 115 may be a clinician examining the subject 205, and may administer the immunotherapy of the same type to the subject 205. Conversely, when the subject 205 is identified as the non-responder to the immunotherapy, the administrator device 115 may display the instruction 170 to discontinue the administration of the immunotherapy. The user of the administrator device 115 may cease administration of the immunotherapy to the subject 205.

The process 230 may be repeated any number of times in connection with administration of various types of immunotherapies to the subject 205. For instance, the determination of the responsiveness score 255 and the identification of the subject 205 as the responder or non-responder may be repeated for the second immunotherapy of the second type. In addition, the instruction 270 may be generated with respect to the multiple types of immunotherapy administered to the subject 205. When the responsiveness score 255 for the first immunotherapy satisfies a first threshold and the responsiveness score 255 for the second immunotherapy satisfies a second threshold, the response classifier 145 may identify the subject 205 as a responder to both the first immunotherapy and the second immunotherapy. The output handler 150 may generate the instruction 270 to continue the administration of at least one of the first immunotherapy or the second immunotherapy, or both. When the responsiveness score 255 for the first immunotherapy satisfies the first threshold and the responsiveness score 255 for the second immunotherapy does not satisfies the second threshold, the response classifier 145 may identify the subject 205 as a responder to the first immunotherapy but a non-responder to the second immunotherapy. The output handler 150 may generate the instruction 270 to continue the administration of the first immunotherapy and discontinue the administration of the second immunotherapy

Continuing on, when the responsiveness score 255 for the first immunotherapy does not satisfy the first threshold and the responsiveness score 255 for the second immunotherapy satisfies the second threshold, the response classifier 145 may identify the subject 205 as a non-responder to the first immunotherapy but a responder to the second immunotherapy. The output handler 150 may generate the instruction 270 to discontinue the administration of the first immunotherapy and continue the administration of the second immunotherapy. When the responsiveness score 255 for the first immunotherapy does not satisfy the first threshold and the responsiveness score 255 for the second immunotherapy does not satisfy the second threshold, the response classifier 145 may identify the subject 205 as a non-responder to both the first immunotherapy and the second immunotherapy. The output handler 150 may generate the instruction 270 to discontinue the administration of both the first immunotherapy and the second immunotherapy

In this manner, the data processing system 105 may process sequencing data in accordance with a set of rules and formulas to objectively determine a significance measure of the administration of the immunotherapy in expanding T cell clones across samples taken from a given subject suffering from cancer. Using the significance measure, the data processing system 105 may more quickly and more accurately identify the subject 205 as responsive or non-responsive to the immunotherapy. With the identification, the data processing system 105 may determine whether the subject 205 is to be administered with additional immunotherapy, and provide an output with the determination. The data processing system 105 can thus provide clinicians with insight as to whether the immunotherapy is effective against the cancer in the subject 205. The output provided by the data processing system 105 may also allow clinicians to readily determine whether to administer additional immunotherapy to the subject 205, thereby reducing the instances of providing ineffective treatment to the subject 205 or improve their health from providing additional immunotherapy treatment. In addition, the data processing system 105 may save consumption of computing resources (e.g., processor and memory) that would have been otherwise wasted in providing less accurate estimates of the impact of such immunotherapies.

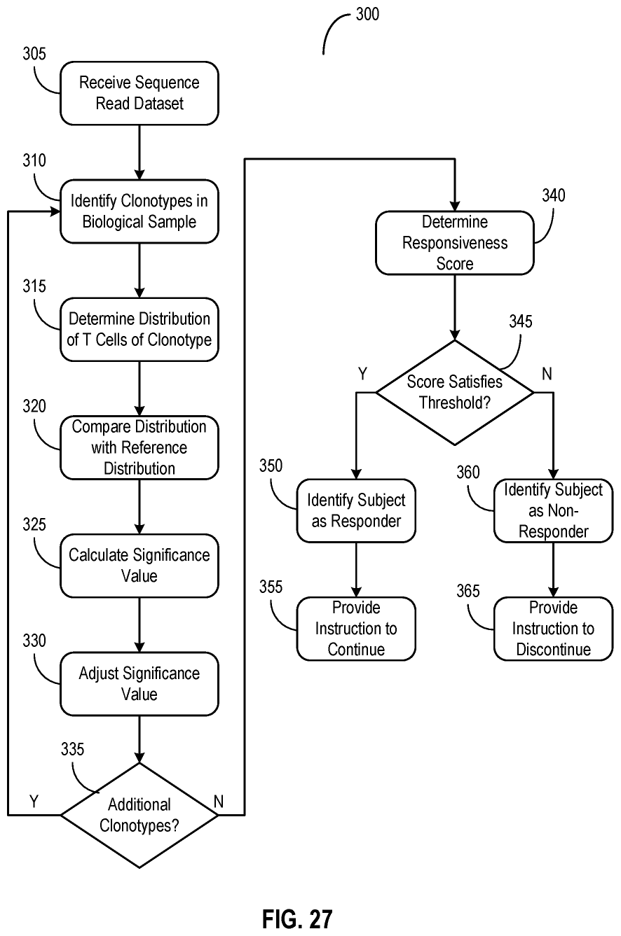

Referring now to FIG. 27, depicted is a flow diagram of a method 300 of determining a likelihood of responsiveness to an immunotherapy in a subject suffering from cancer. The method 300 may be implemented or performed using any of the components described herein, such as the data processing system 100 detailed herein conjunction with FIG. 25 or the server system 500 detailed herein in conjunction with FIG. 29. Under the method 300, a computing system may receive a sequence read dataset of a biological sample from a subject administered with an immunotherapy (305). The computing system may identify a clonotype in the biological sample from the sequence read dataset (310). The computing system may determine a distribution of T cells of the clonotype (315). The computing system may compare the distribution of T cells of the clonotype with a reference distribution of T cells of the same clonotype in a reference biological sample (320). The computing system may calculate a significance value based on the comparison (325). The computing system may adjust the significance value (330).

The computing system may determine whether there are any additional clonotypes to evaluate (335). If there are additional clonotypes, the computing system may identify the next clonotype in the biological sample to repeat from step (310). If there are no more clonotypes to evaluate, the computing system may determine a responsiveness score for the subject (340). The computing system may determine whether the responsiveness score satisfies a threshold (345). If the responsiveness score satisfies the threshold, the computing system may identify the subject as a responder to the immunotherapy (350). The computing system may also provide an instruction to continue administration of the immunotherapy (355). Otherwise, if the responsiveness score does not satisfy the threshold, the computing system may identify the subject as a non-responder to the immunotherapy (360). The computing system may provide an instruction to discontinue the administration of the immunotherapy (365).

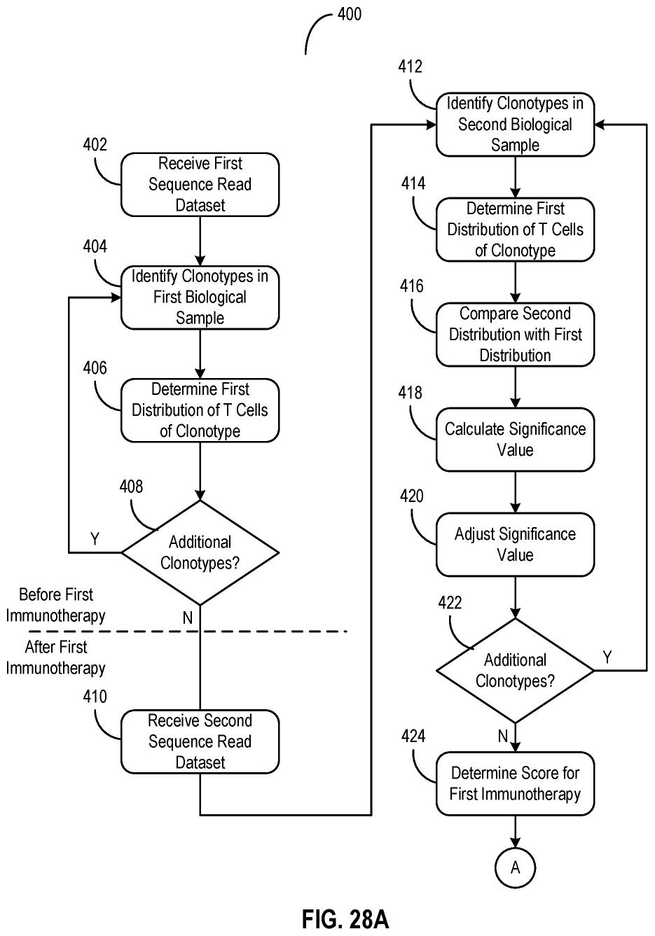

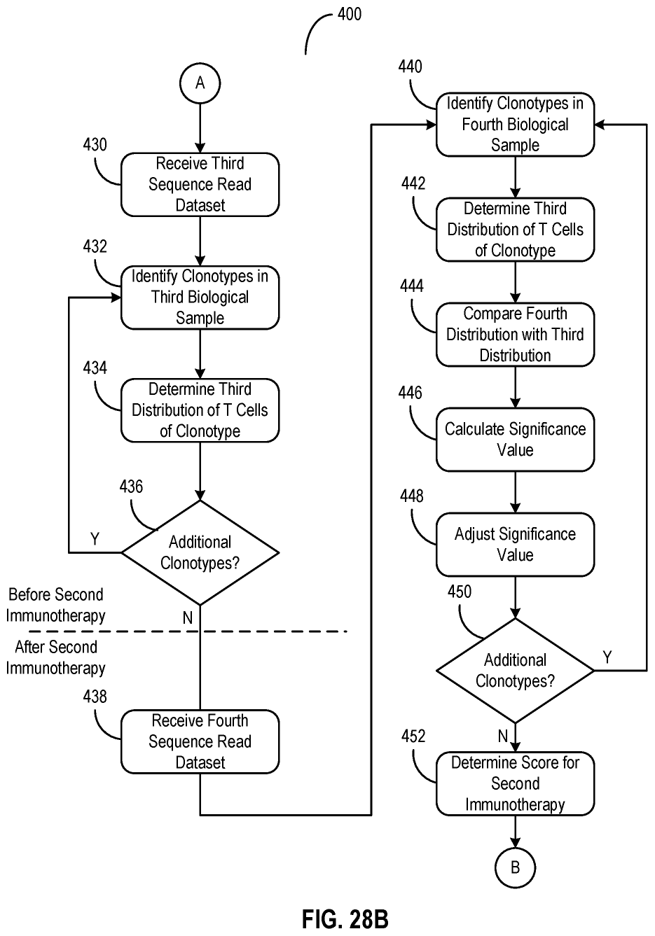

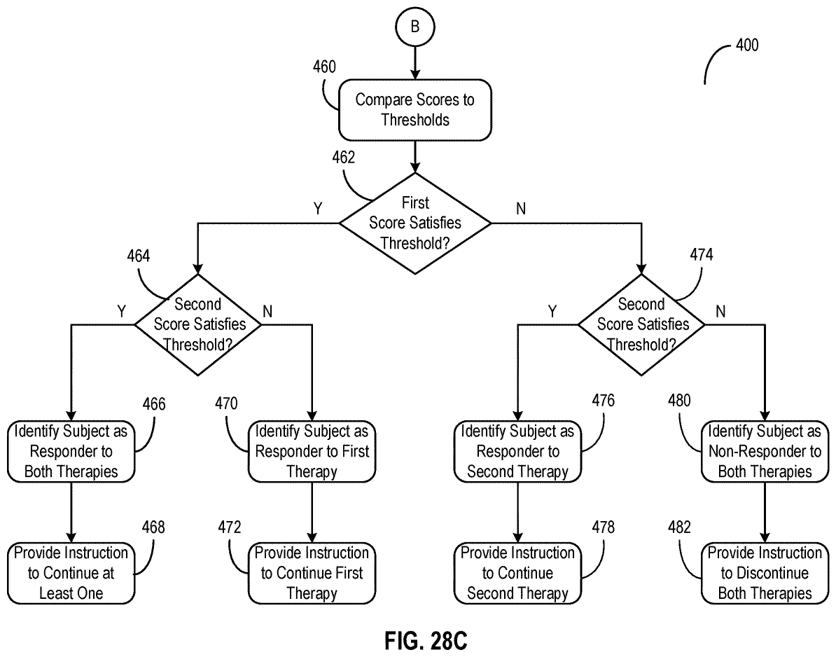

Referring now to FIGS. 28 A-28C, depicted are flow diagrams of a method 400 of monitoring responsiveness to at least one type of immunotherapy in a subject suffering from cancer. The method 400 may be implemented or performed using any of the components described herein, such as the data processing system 100 detailed herein conjunction with FIG. 25 or the server system 500 detailed herein in conjunction with FIG. 29. Starting from FIG. 28A, under the method 400, a computing system may receive a first sequence read dataset of a first biological sample from a subject prior to administration of a first type of immunotherapy (402). The computing system may identify a clonotype in the first biological sample from the sequence read dataset (404). The computing system may determine a distribution of T cells of the clonotype (406). The computing system may determine whether there are any additional clonotypes to evaluate (408). If there are additional clonotypes, the computing system may identify the next clonotype in the first biological sample to repeat from step (404).

If there are no more clonotypes to evaluate, the computing system may wait and receive a second sequence read dataset of a second biological sample from the subject subsequent to administration of the first type of immunotherapy (410). The computing system may identify a clonotype in the biological sample from the second sequence read dataset (412). The computing system may determine a distribution of T cells of the clonotype (414). The computing system may compare the distribution of T cells of the clonotype with the distribution of T cells of the same clonotype in the second biological sample (416). The computing system may calculate a significance value based on the comparison (418). The computing system may adjust the significance value (420). The computing system may determine whether there are any additional clonotypes to evaluate in the second biological sample (422). If there are additional clonotypes, the computing system may identify the next clonotype in the second biological sample to repeat from step (412). If there are no more clonotypes to evaluate, the computing system may determine a responsiveness score for the subject to the first type of immunotherapy (424).

Moving onto FIG. 28B, the computing system may receive a first sequence read dataset of a third biological sample from the subject prior to administration of a second type of immunotherapy (430). The computing system may identify a clonotype in the third biological sample from the sequence read dataset (432). The computing system may determine a distribution of T cells of the clonotype (434). The computing system may determine whether there are any additional clonotypes to evaluate (436). If there are additional clonotypes, the computing system may identify the next clonotype in the third biological sample to repeat from step (432).

If there are no more clonotypes to evaluate, the computing system may wait and receive a fourth sequence read dataset of a fourth biological sample from the subject subsequent to administration of the second type of immunotherapy (438). The computing system may identify a clonotype in the biological sample from the fourth sequence read dataset (440). The computing system may determine a distribution of T cells of the clonotype (442). The computing system may compare the distribution of T cells of the clonotype with the distribution of T cells of the same clonotype in the fourth biological sample (444). The computing system may calculate a significance value based on the comparison (446). The computing system may adjust the significance value (448). The computing system may determine whether there are any additional clonotypes to evaluate in the fourth biological sample (450). If there are additional clonotypes, the computing system may identify the next clonotype in the fourth biological sample to repeat from step (440). If there are no more clonotypes to evaluate, the computing system may determine a second responsiveness score for the subject to the second type of immunotherapy (452).

Continuing onto FIG. 28C, the computing system may compare the responsiveness scores to threshold (460). The computing system may determine whether the first responsiveness score satisfies the threshold (462). When the first responsiveness score satisfies the threshold, the computing system may determine whether the second responsiveness satisfies the threshold (464). If the second responsiveness score also satisfies the threshold, the computing system may identify the subject as a responder to both the first type of immunotherapy and the second type of immunotherapy (466). The computing system may provide an instruction to continue administration of at least one of the first type or the second type of immunotherapy (468). If the second responsiveness score does not satisfy the threshold, the computing system may identify the subject as a responder to the first type of immunotherapy but not the second type of immunotherapy (470). The computing system may provide an instruction to continue administration of the first type of immunotherapy (472). The computing system may also provide an instruction to discontinue administration of the second type of immunotherapy.

When the first responsiveness score does not satisfy the threshold, the computing system may determine whether the second responsiveness satisfies the threshold (474). If the second responsiveness score also satisfies the threshold, the computing system may identify the subject as a responder to the second type of immunotherapy but not the first type of immunotherapy (476). The computing system may provide an instruction to continue administration of the second type of immunotherapy (478). The computing system may also provide an instruction to discontinue administration of the first type of immunotherapy. If the second responsiveness score also does not satisfy the threshold, the computing system may identify the subject as a non-responder to both the first type of immunotherapy and the second type of immunotherapy (480). The computing system may provide an instruction to discontinue administration of both the first type and the second type of immunotherapy (482).

### Computing and Network Environment

Various operations described herein can be implemented on computer systems. FIG. 29 shows a simplified block diagram of a representative server system 500, client computing system 514, and network 526 usable to implement certain embodiments of the present disclosure. In various embodiments, server system 500 or similar systems can implement services or servers described herein or portions thereof. Client computing system 514 or similar systems can implement clients described herein. The system 600 described herein can be similar to the server system 500. Server system 500 can have a modular design that incorporates a number of modules 502 (e.g., blades in a blade server embodiment); while two modules 502 are shown, any number can be provided. Each module 502 can include processing unit(s) 504 and local storage 506.

Processing unit(s) 504 can include a single processor, which can have one or more cores, or multiple processors. In some embodiments, processing unit(s) 504 can include a general-purpose primary processor as well as one or more special-purpose co-processors such as graphics processors, digital signal processors, or the like. In some embodiments, some or all processing units 504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 504 can execute instructions stored in local storage 506. Any type of processors in any combination can be included in processing unit(s) 504.

Local storage 506 can include volatile storage media (e.g., DRAM, SRAM, SDRAM, or the like) and/or non-volatile storage media (e.g., magnetic or optical disk, flash memory, or the like). Storage media incorporated in local storage 506 can be fixed, removable or upgradeable as desired. Local storage 506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 504 need at runtime. The ROM can store static data and instructions that are needed by processing unit(s) 504. The permanent storage device can be a non-volatile read-and-write memory device that can store instructions and data even when module 502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

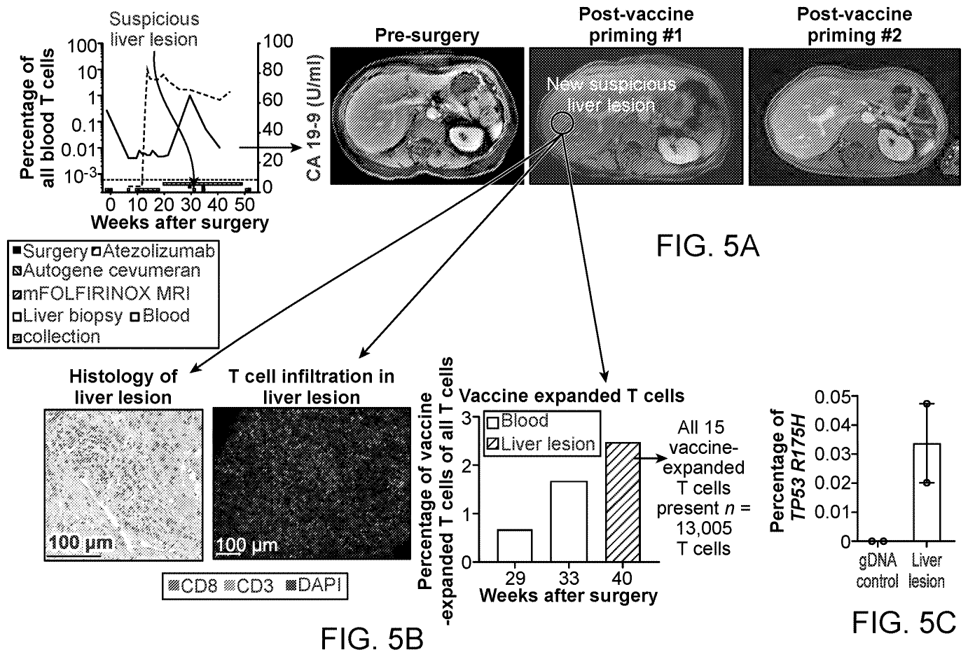

In some embodiments, local storage 506 can store one or more software programs to be executed by processing unit(s) 504, such as an operating system and/or programs implementing various server functions such as functions of the system 500 of FIG. 5 or any other system described herein, or any other server(s) associated with system 500 or any other system described herein.

“Software” refers generally to sequences of instructions that, when executed by processing unit(s) 504 cause server system 500 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 504. Software can be implemented as a single program or a collection of separate programs or program modules that interact as desired. From local storage 506 (or non-local storage described below), processing unit(s) 504 can retrieve program instructions to execute and data to process in order to execute various operations described above.

In some server systems 500, multiple modules 502 can be interconnected via a bus or other interconnect 508, forming a local area network that supports communication between modules 502 and other components of server system 500. Interconnect 508 can be implemented using various technologies including server racks, hubs, routers, etc.

A wide area network (WAN) interface 510 can provide data communication capability between the local area network (interconnect 508) and the network 526, such as the Internet. Technologies can be used, including wired (e.g., Ethernet, IEEE 502.3 standards) and/or wireless technologies (e.g., Wi-Fi, IEEE 502.11 standards).

In some embodiments, local storage 506 is intended to provide working memory for processing unit(s) 504, providing fast access to programs and/or data to be processed while reducing traffic on interconnect 508. Storage for larger quantities of data can be provided on the local area network by one or more mass storage subsystems 512 that can be connected to interconnect 508. Mass storage subsystem 512 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 512. In some embodiments, additional data storage resources may be accessible via WAN interface 510 (potentially with increased latency).

Server system 500 can operate in response to requests received via WAN interface 510. For example, one of modules 502 can implement a supervisory function and assign discrete tasks to other modules 502 in response to received requests. Work allocation techniques can be used. As requests are processed, results can be returned to the requester via WAN interface 510. Such operation can generally be automated. Further, in some embodiments, WAN interface 510 can connect multiple server systems 500 to each other, providing scalable systems capable of managing high volumes of activity. Other techniques for managing server systems and server farms (collections of server systems that cooperate) can be used, including dynamic resource allocation and reallocation.

Server system 500 can interact with various user-owned or user-operated devices via a wide-area network such as the Internet. An example of a user-operated device is shown in FIG. 5 as client computing system 514. Client computing system 514 can be implemented, for example, as a consumer device such as a smartphone, other mobile phone, tablet computer, wearable computing device (e.g., smart watch, eyeglasses), desktop computer, laptop computer, and so on.

For example, client computing system 514 can communicate via WAN interface 510. Client computing system 514 can include computer components such as processing unit(s) 516, storage device 518, network interface 520, user input device 522, and user output device 524. Client computing system 514 can be a computing device implemented in a variety of form factors, such as a desktop computer, laptop computer, tablet computer, smartphone, other mobile computing device, wearable computing device, or the like.

Processing unit(s) 516 and storage device 518 can be similar to processing unit(s) 504 and local storage 506 described above. Suitable devices can be selected based on the demands to be placed on client computing system 514; for example, client computing system 514 can be implemented as a “thin” client with limited processing capability or as a high-powered computing device. Client computing system 514 can be provisioned with program code executable by processing unit(s) 516 to enable various interactions with server system 500.

Network interface 520 can provide a connection to the network 526, such as a wide area network (e.g., the Internet) to which WAN interface 510 of server system 500 is also connected. In various embodiments, network interface 520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 522 can include any device (or devices) via which a user can provide signals to client computing system 514; client computing system 514 can interpret the signals as indicative of particular user requests or information. In various embodiments, user input device 522 can include any or all of a keyboard, touch pad, touch screen, mouse or other pointing device, scroll wheel, click wheel, dial, button, switch, keypad, microphone, and so on.

User output device 524 can include any device via which client computing system 514 can provide information to a user. For example, user output device 524 can include a display to display images generated by or delivered to client computing system 514. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 524 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 504 and 516 can provide various functionality for server system 500 and client computing system 514, including any of the functionality described herein as being performed by a server or client, or other functionality.

It will be appreciated that server system 500 and client computing system 514 are illustrative and that variations and modifications are possible. Computer systems used in connection with embodiments of the present disclosure can have other capabilities not specifically described here. Further, while server system 500 and client computing system 514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### EXAMPLES

Examples are provided below to facilitate a more complete understanding of the present technology. The following examples illustrate the exemplary modes of making and practicing the present technology. However, the scope of the present technology is not limited to specific embodiments disclosed in these Examples, which are for purposes of illustration only, since alternative methods can be utilized to obtain similar results.

Aspects of the present technology are drawn to a new method to identify T cell clones that expand in response to immunotherapies. Recent advances in next generation sequencing allow recognition of T cell clones by sequencing a T cell's unique antigen recognition domain in its receptor. Though this sequencing methodology is widely used, specific methods to accurately identify clones that expand or decline with specific therapies, to thereby identify specific clones that change with treatment, remains unknown.

Embodiments as described herein identifies T cell clones that expand in response to immunotherapies (for example, mRNA vaccines, checkpoint blockade immunotherapies). We further validate the specificity of these clones to the specific antigens in vaccines, further confirming the validity of this method.

Without wishing to be bound by theory, embodiments as described herein can be broadly adopted to identify clonal change across a variety of medical and scientific applications.

### Example 1: Materials and Methods

### Mutation Identification and Neoantigen Selection

DNA was extracted from normal peripheral blood mononuclear cells. DNA and RNA were extracted from tumors. Expressed non-synonymous mutations and HLA type were identified by whole-exome sequencing of patient specific tumor/normal pairs and tumor RNA-sequencing. Neoantigens were bioinformatically predicted and ranked by immunogenicity using the autogene cevumeran Genentech Recurrent Attention Framework (GRAF) deep learning model.

### Production Benchmarks

We set a priori benchmarks from surgery to vaccine manufacture:
(1) transport specimen from operating room to pathology in ≤5 minutes
(2) fix specimen in formalin and embed in paraffin in ≤15 minutes
(3) select blocks for vaccine production in ≤2 days
(4) ship in ≤72 hours
(5) produce vaccines in ≤6 weeks
(6) administer first dose of the vaccine in ≤9 weeks

### Autogene Cevumeran Manufacturing

For every patient, individualized mRNA neoantigen vaccines were manufactured under GMP conditions containing 2 mRNA strands, each strand encoding up to 10 MHC-I and MHC-II neoepitopes, formulated in ˜400 nm diameter lipoplex nanoparticles 1 comprised of the synthetic cationic lipid (R)-N,N,N-trimethyl-2,3-dioleyloxy-1-propanaminium chloride (DOTMA) and the phospholipid 1,2-dioleoyl-sn-glycero-3-phosphatidylethanolamine (DOPE) to enable intravenous (IV) delivery.

### Immune Response

### TCR Vβ Sequencing

We prepared genomic DNA from bulk PBMCs, or purified T cells using a Qiagen® DNA extraction kit according to the manufacturer's instructions. We quantified samples using the DropsenseR 96 and diluted to standard concentrations for library preparation. We generated sample data using the immunoSEQR Assay (Adaptive Biotechnologies, Seattle, WA). Briefly, the somatically rearranged TCRβ CDR3 was amplified from^{2,3} genomic DNA using a two-step, amplification bias-controlled multiplex PCR approach. The first PCR consists of forward and reverse amplification primers specific for every known V and J gene segment, and amplifies the hypervariable CDR3 of the immune receptor locus. The second PCR adds a proprietary barcode sequence and Illumina® adapter sequences.4 In addition, reference gene primers are included in the PCR reaction to quantify total nucleated cells that can be sequenced, and accurately measure the fraction of T cells in each sample. CDR3 and reference gene libraries were sequenced on an Illumina® instrument according to the manufacturer's instructions. Raw sequence reads were demultiplexed according to Adaptive's proprietary barcode sequences. Demultiplexed reads were further processed to remove adapter and primer sequences; identify and remove primer dimer, germline, and other contaminant sequences. The filtered data is clustered using both the relative frequency ratio between similar clones and a modified nearest-neighbor algorithm, to merge closely related sequences to correct for technical errors introduced through PCR and sequencing. The resulting sequences were sufficient to annotate the V, D, and J genes and the N1, N2 regions constituting each unique CDR3 and the translation of the encoded CDR3 amino acid sequence. Gene definitions were based on annotation in accordance with the IMGT database (www.imgt.org). The set of observed biological TCRB CDR3 sequences were normalized to correct for residual multiplex PCR amplification bias and quantified against a set of synthetic TCRB CDR3 sequence analogues.³

### T Cell Clone Definition

We identified and tracked T cell clones by their β chain sequence (TRB), defined as the nucleotide CDR3 sequence (including the conserved C and F residues) and a deterministic V and J gene alignment. For T cells identified by single cell sequencing, we similarly defined clones by the TRB sequence to map clones to paired TCR Vβ sequencing. Due to the higher entropy of the nucleotide CDR3 sequence generation probability distribution,⁵ we used nucleotide instead of amino acid CDR3 sequences to minimize the chance of conflating two different T cell clones (different original VDJ recombination events). This becomes critical to differentiate clones that may have different α chain (TRA) sequences which are unobserved in the bulk TCR Vβ sequencing.

We used the provided deterministic V and J alignments from Adaptive Biotechnologies (for bulk TCR Vβ sequencing) and 10× (for single cell sequencing).

### T Cell Clone Frequency Estimation

For a given sample of bulk TRB sequences, we estimated the total number of effective cells sequenced, N, as the summation of all productive (in-frame, no stop codons) reads. We excluded non-productive reads, as they must necessarily be recombined CDR3s from silenced alleles (we do not model the fraction of productive reads from silenced alleles here and assume them to be a small correction). We estimated a T cell clone x's cell count nₓ in a sample as the number of reads corresponding to the clone as defined above (V/J gene and nucleotide CDR3 sequence). We thus estimated the frequency of clone x as

$${f}_{x} = \frac{{n}_{x}}{N} .$$

For the purpose of visualization, if we did not observe a clone in a sample, we use a pseudo-frequency of

$$\frac{1}{3 N}$$

if plotting a trajectory with multiple samples, we use the largest N over the samples) and indicated this observation threshold as dotted black lines. We computed the aggregate frequency of several clones x∈X in a similar fashion using an aggregate count nₓ=Σ_{x∈X} nₓ and used the same convention for a pseudo-frequency if nₓ=0.

### T Cell Clone Significance Determination:

We took a statistically conservative approach to minimize false positive identification of expanded T cell clones. To this end, to calculate treatment expanded T cell clones, we used a statistically significance threshold of p_{adj}<0.001, where the P value is adjusted by the Bonferroni correction (p_{adj}=p×#T cell clones) to account for the large number of T cell clones that are screened.

### T Cell Clone Expansion Criteria

To identify treatment expanded T cell clones, we used an adapted Fisher exact test, and computed P values for expanded T cell clones using a two-tailed adapted exact Fisher test for a 2-fold increase in a T cell clone between any two samples.

We implemented this by rescaling the repertoire size of the initial sample by half, to effectively reduce the sample size and the number of cells not belonging to the clone in question. We computed this as a Fisher exact test (implemented from scipy.stats.fisher_exact) on the following categorical table:

| | # cells ∈ T | Rescaled # cells ∉ | Rescaled # cells |
|---|---|---|---|
| | cell clone x | T cell clone x | in sample |
| Baseline sample | nₓ | [N/2] − nₓ | [N/2] |
| Comparative | mₓ | M − mₓ | M |
| sample | | | |

Clones which had a fold change <2

$$( i . e . \frac{{m}_{x}}{M} < 2 \times \frac{{n}_{x}}{N} )$$

were assigned a P value of 1. These P values were then adjusted using the Bonferroni correction: p_{adj}=p×|N∩∪M| where |N∪M| designates the number of unique clones in the union of the two samples.

We applied this T cell clone expansion P value in the following two contexts to determine if either atezolizumab or autogene cevumeran induced an immunologic response.

### Atezolizumab Response:

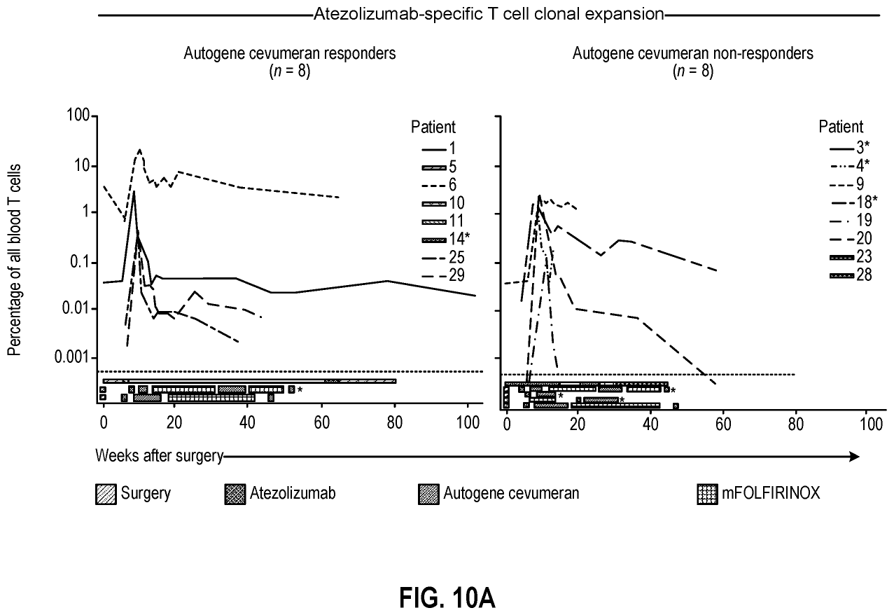

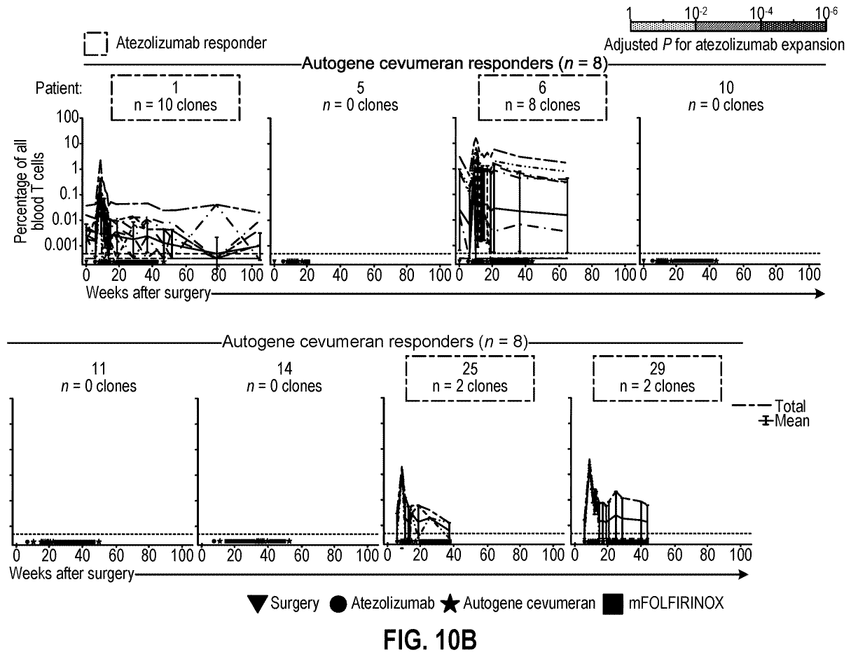

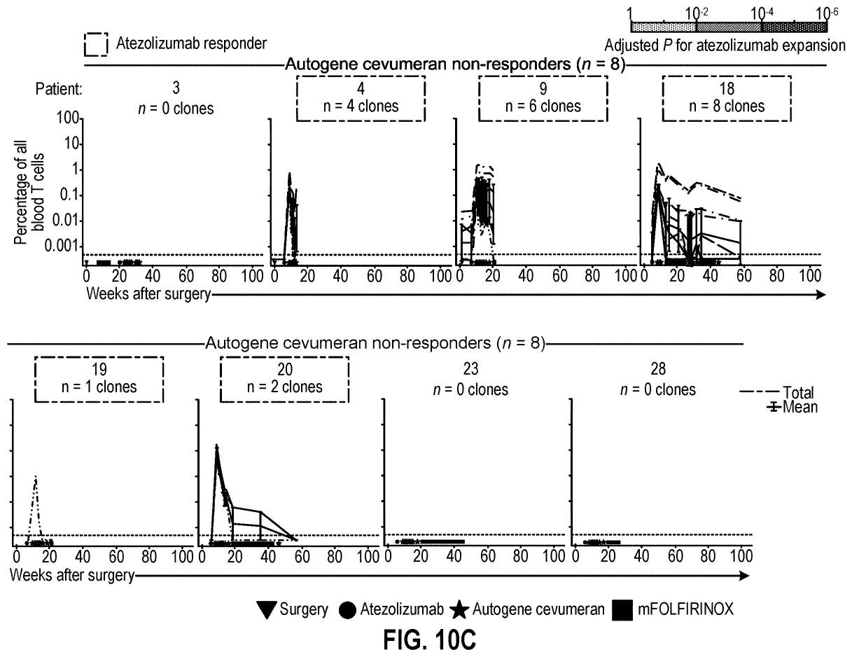

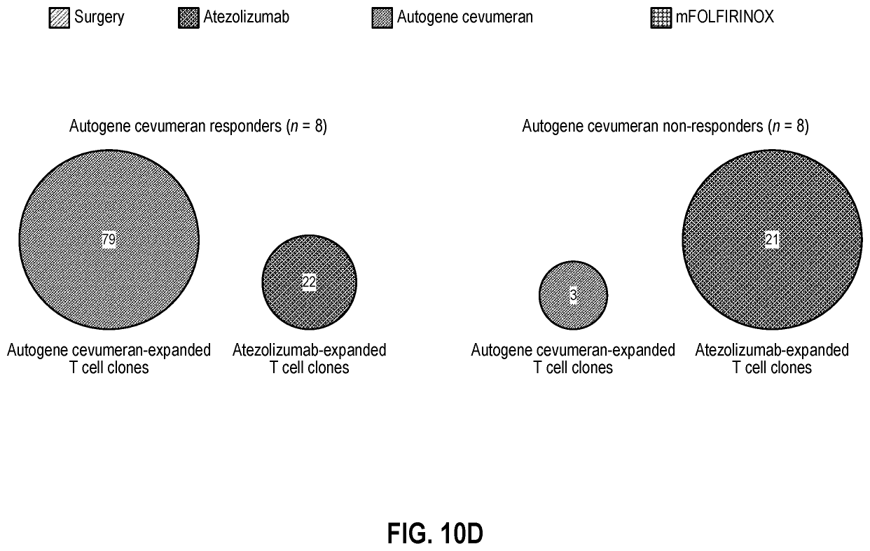

To determine if atezolizumab expanded T cell clones (FIGS. 10A-10D), we compared the number of cells of a particular T cell clone in a blood sample taken on the day of but prior to atezolizumab administration, to the number of cells of that T cell clone in a blood sample taken on the day of but prior to the first dose of autogene cevumeran.

We then adjudicated a patient to have a response to atezolizumab if any T cell clone was found to be significantly expanded (p_{adj}<0.001) according to the above outlined expansion criterion.

### Autogene Cevumeran Response:

We assessed if autogene cevumeran expanded T cells that recognize neoantigens in patients' individualized mRNA vaccines by two independent assays.

### Assay 1: TCR Vβ Clone Tracking:

To determine if autogene cevumeran expanded T cell clones, we further imposed two criteria:
a) a quality control requirement that a T cell clone must have a minimum of 3 reads in at least two samples, and
b) the T cell clone must not be observed pre-vaccination (0 cells in all samples taken through the day of, but prior to, the first dose of autogene cevumeran).

Then, for clones that passed this criteria (as defined above), we compared the number of cells of a particular T cell clone in a blood sample taken post-atezolizumab and pre-vaccination, to the number of cells of that T cell clone in any blood sample taken until the day of, but prior to, the first dose of mFOLFIRINOX. We further assigned an expansion P value as defined as the minimum adjusted expansion P value for all samples, further adjusted by a Bonferroni correction for the number of samples the expansion P values were computed for.

We then adjudicated a patient to have a response to autogene cerumevan by assay 1 if any T cell clone significantly expanded (p_{adj}<0.001) according to the above outlined expansion criteria.

### Assay 2: IFNγ ELISPOT.

Multiscreen filter plates (Merck Millipore), precoated with antibodies specific for IFNγ (Mabtech), were washed with phosphate-buffered saline (PBS) and blocked with X-VIVO 15 (Lonza) containing 2% human serum albumin (CSL-Behring) for 1-5 h. Next, 3×105 effector cells per well were stimulated for 16-20 h with peptide pools per target. Cryopreserved PBMCs were subjected to ELISpot after a resting period of 2-5 h at 37° C. All tests were performed in duplicate and included anti-CD3 (Mabctech) as a positive control. Bound IFNγ was visualized using a secondary antibody directly conjugated with alkaline phosphatase (ELISpotPro kit, Mabtech). Next, plates were incubated with BCIP/NBT (5-bromo-4-chloro-3′-indolyl phosphate and nitro blue tetrazolium) substrate (ELISpotPro kit, Mabtech. Plates were scanned using an AID Classic Robot ELISPOT Reader and analyzed by AID ELISPOT 7.0 software (AID Autoimmun Diagnostika). Peptide-stimulated spot counts were compared to effectors incubated with medium only as negative control using an in-house ELISpot data analysis tool, based on two statistical tests (distribution-free resampling). To account for varying sample quality reflected in the number of spots in response to anti-CD3 antibody stimulation, we applied a normalization method that enabled direct comparison of spot counts and strength of response between individuals, as described previously.⁶⁻⁸

### Neoantigen Quality and Vaccine Response

To model neoantigen quality, we adapt our previously described model^{9,10} that identified spontaneously targeted neoantigens in tumors, to now identify optimally immunogenic neoantigens suited for vaccines. Specifically, without wishing to be bound by theory, the immunogenicity (or quality) of a neoantigen is the product of two components. The first component—the non-self recognition potential G of a neoantigen—is the inherent immunogenicity of the neopeptide. The second component—the self-discrimination potential H—models whether a neoantigen's cognate T cells avoid negative thymic selection, to thus render neoantigen recognition less constrained by self toleration. Previous versions of our quality model estimated the non-self recognition potential G of a neopeptide using sequence homology (as determined by soft max rescaling of BLAST alignment) to the immunogenic infectious disease-derived epitopes in the Immune Epitope Data Base (IEDB). Self-discrimination was estimated as a sum of two free discrimination energies between the neoantigen and its wildtype peptide, one for differential MHC presentation, the other for differential T cell cross reactivity:

$$D = log ( \frac{{k}_{D}^{WT}}{{k}_{D}^{MT}} ) + \vert log ( \frac{{EC}_{50}^{MT}}{{EC}_{50}^{WT}} ) \vert ,$$

where k_{D} is the HLA specific peptide-MHC affinity (as estimated by netMHC 3.4), and EC₅₀ is the concentration for 50% activation for an avidity curve with the neopeptide and its cognate T cell clone.^{9,10} Furthermore, in previous studies, we restricted our definition of minimal epitopes to consider to only 9-mers, the most common length of MHC-I bound peptides, predicted to bind to the HLA of the patient with a cutoff of 500 nM.

To now extend the notion of the cross-reactivity or epitope distance beyond the single substitution case as previously described,^{9,10} we now make an independent site approximation by modeling the cross-reactivity distance, d_{c}, between two 9-mer epitopes, p^{A} and p^{B} as:

$${d}_{c} ( {p}^{A} , {p}^{B} ) = {\sum }_{i = 1}^{9} {d}_{i} {M}_{{p}_{i}^{A} , {p}_{i}^{B}} \approx \vert log ( \frac{{EC}_{50} ( {p}^{A} )}{{EC}_{50} ( {p}^{B} )} ) \vert$$

where dᵢ is a scaling weight for position i and M is the substitution matrix inferred from.¹⁰ This extension allows us to replace the estimation of the non-self recognition potential R of a neopeptide from sequence homology using BLAST with epitope distance.

We now take as our two components, in the context of vaccination, how far a neopeptide is from the germline and how close it is to known antigenic IEDB epitopes. For a given 9-mer minimal neoepitope, p^{MT} we define the quality of the 9-mer as:

$$Q ( {p}^{MT} ) = {d}_{c} ( {p}^{MT} , {p}^{WT} ) - \begin{matrix}min \\ {p}^{a} ∈ {P}_{IEDB}\end{matrix} {d}_{c} ( {p}^{M T} , {p}^{a} )$$

where P_{IEDB} is the collection of all 9-mers sequences and sub-sequences of IEDB epitopes.

We define the quality of a neopeptide as the average quality over the two highest quality 9-mer sub-sequences that include the substituted residue and are predicted binders (threshold of 4000 nM) to the individual's HLA type. As a vaccine can induce neoantigen expression well in excess of endogenous expression in a tumor, we thus drop the differential MHC presentation term, and relax our MHC binding cutoff.

To check if this neoantigen quality model has any predictive power to determine the immunogenicity of neoantigenic peptides included in the vaccines used in this study, we classify the neopeptides from the n=8 immune responders as derived from immunogenic or non-immunogenic neoantigens according to the ELISpot assay. Immunogenicity was unable to be established for 7 of the neoantigens from CTMS-25 and are excluded from the analysis. We use neoantigens only from immune responding patients to ensure that lack of an immunologic response to a neoantigen reflects non-immunogenicity and not general vaccine failure. This generates 23 immunogenic neoantigens out of a total of 99 screened neoantigens from n=8 immune responders. After excluding neoantigens with no predicted minimal epitope binders, we have a final cohort of 22 reactive neopeptides out of a total of 79.

Cell culture

We purified patient peripheral blood mononuclear cells (PBMCs) from blood samples by density centrifugation over Ficoll-Paque Plus (GE Healthcare, IL, USA). We purified healthy donor PBMCs from buffy coats (New York Blood Center, NY, USA) and isolated T cells using a Pan-T cell isolation kit (Miltenyi Biotech, Germany). We activated T cells with CD3/CD28 beads (Thermo Fisher, MA, USA) with IL-7 (3000 IU/ml) and IL-15 (100 IU/ml) (Miltenyi Biotec, Germany), and transduced T cells on day 2 post activation. Virus-producing cell lines (H29 and RD114-envelope producers) were previously described.^{11,12} We cultured T cells and K562 cells in RPMI media supplemented with 10% fetal bovine serum (FBS, Nucleus Biologics, CA, USA), 100 U/ml Penicillin/Streptomycin (Thermo Fisher Gibco, MA, USA), and 2 mM L-Glutamine (Thermo Fisher Gibco, MA, USA). We cultured patient PBMCs with RPMI media supplemented with 10% FBS, 1 mM Sodium pyruvate, 2 mM L-Glutamine, non-essential amino acids, 2-mercaptoethanol (MSK media preparation core facility). We cultured H29, RD114-envelope producers, and Phoenix-AMPHO in DMEM media supplemented with 10% FBS (Nucleus Biologics, CA, USA), 100 U/ml Penicillin/Streptomycin (Thermo Fisher Gibco, MA, USA), and 2 mM L-Glutamine (Thermo Fisher Gibco, MA, USA).

### In Vitro Stimulation and T Cell Clone Specificity to Peptides

We resuspended peptides (Genscript, NJ, USA) in DMSO at 10 mg/ml and stored at −80° C. We restimulated peptides in vitro as previously described with minor modifications.¹³ In brief, we cultured 1×10⁶ PBMCs in a 48-well plate with individual (10 mg/ml) or pooled (1-5 mg/ml per peptide) peptides on day 1. We added IL-2 (100 U/ml) and IL-15 (10 ng/ml) on day 2 and every subsequent 2-3 days. On day 7, we restimulated cells with peptides and incubated with CD107a antibody (clone H4A3, PE, BD Biosciences, CA, USA) for 1 h at 37° C. After 1 h, we added protein transport inhibitor containing Monensin (BD Biosciences, CA, USA) and incubated for 4 h at 37° C. We then stained the cells for additional surface or intracellular markers as per the manufacturer guidelines, and either analyzed or purified cells based on CD107a surface expression, or analyzed cells based on intracellular cytokine expression.

### T Cell Clone Specificity to Stimulated Peptides

To determine if a T cell clone is specifically stimulated by the peptide pool, we sorted and identified T cell clones in CD107a⁻ and CD107a⁺ fractions post-peptide stimulation as described above. We then determined a peptide specificity stimulation P value for each T cell clone using a one-tailed binomial test P value (implementing the scipy.stats.binom test) with a 0.2 threshold (specifically, significance with respect to at least 20% of a clone being CD107a⁺ as opposed to CD107a⁻). We adjusted P values using a Bonferroni correction and determined significance at a p_{adj}<0.001 threshold. We included a DMSO control to identify nonspecifically stimulated T cell clones. Of all patients tested, only one nonspecific clone was identified (CTMS-10) as nonspecifically stimulated in DMSO and both screened peptide pools. This clone was thus excluded as a peptide-specific clone.

### HLA Cloning and Transduction

We cloned the HLA alleles into an SFG g-retroviral vector¹⁴, and sequence-verified all plasmids (Genewiz, NJ, USA). We transfected Phoenix-AMPHO cells with the plasmids using MegaTran 2.0 (OriGene, MD, USA). We collected virus-containing supernatants 48 h after transfection, added Polybrene (EMD Millipore, MA, USA), and spinoculated K562 cells for 2 h at 2400 rpm at 33° C. Seventy-two hours post-transduction, we sorted HLA⁺ K562 cells using an Aria Cell sorter (BD Biosciences, CA, USA).

### TCR Cloning, Transduction, and Peptide Stimulations

We constructed TCR fragments as previously described.¹⁵ Briefly, we isolated TRB V-D-J and TRA V-J sequences from purified, sequenced single T cells, and fused the TRB V-D-J and TRA V-J sequences to modified mouse constant TRB and TRA chain sequences,¹⁵ respectively, to prevent mispairing of transduced TCRβ with the endogenous TCRs.¹⁶ Briefly, we joined TRB and TRA chains with a furin SGSG P2A linker, cloned the TCR constructs into an SFG g-retroviral vector¹⁴, and sequence-verified all plasmids (Genewiz, NJ, USA). We transfected H29 cells (gpg29 fibroblasts) with retrovirus vectors using calcium phosphate, and produced VSV-G pseudo-typed retroviruses.11 We next used Polybrene (Sigma, MO, USA) and viral-containing supernatants to generate stable RD114-enveloped producer cell lines.¹² We collected and concentrated virus-containing supernatants using Retro-X™ Concentrator (Takara, Japan). We then coated non-tissue culture treated 6-well plates with Retronectin (Takara, Japan), plated a titrated viral quantity to 3×10⁶ activated T cells per well, centrifuged cells for 1 h at room temperature at 300 g, and used transduced T cells between day 7-14 post-transduction or cryopreserved them for future use. To stimulate TCR-transduced T cells with peptides, we pulsed 2.5×10⁵ HLA-transduced K562 cells (antigen presenting cells, APCs) in a 96-well U-bottom plate for 1 h at 37° C. with the indicated peptides at the indicated concentrations. After 1 h, we washed the peptide by centrifugation, and added 5×10⁴ TCR or mock (control) transduced T cells per well. We then measured CD137 (4-1BB) expression on CD8⁺ mTCR⁺ T cells 24 h later.

### Flow Cytometry

We let patient PBMCs rest overnight at 37° C. and 5% CO2 before16 staining. We defined TCR-transduced CD8⁺ T cells as live, CD3⁺, CD8⁺, mTCR⁺ cells. We stained cells with the following antibodies: from Biolegend (CA, USA)—CD62L (clone DREG-56, BV510), CD56 (clone HCD56, BV605), CD4 (clone OKT4, BV650), CD19 (clone HIB19, BV711), FoxP3 (clone 206D, PE), CD3 (clone SK-7, PE-Cy7), CD8 (clone SK1, FITC or Alexa Fluor 700), CD45RA (clone HI100, APC), CD45 (clone 2D1, Alexa Fluor 700), CD39 (clone A1, BV421), LAG-3 (clone 11C3C65, PerCP-Cy5.5), CD366 (clone F38-2E2, APC-Cy7) CD11c (clone S-HCL-3, BV421), HLA-DR (clone L243, BV785), CD14 (clone HCD14, PE), CD11b (clone ICRF44, APC), IFNγ (clone 4S.B3, BV421), mTRB (clone H57-597, PE-Cy5), CD137 (clone 4B4-1, PE), HLA-A,B,C (clone W6/23, APC), and Zombie Red Fixable Viability Kit (catalog #423110); from BD Biosciences (CA, USA)—PD-1 (clone EH12.1, BV786), TNFα (clone MAb11, APC), CD107a (clone H4A3, PE), CD56 (clone NCAM16.2, BV786), and DAPI (catalog #564907); from ThermoFisher Scientific (MA, USA)—Ki-67 (clone SolA15, PE-Cy5). We stained cells using antibody cocktails in the dark at 4° C., washed, and analyzed on a FACS LSR Fortessa (BD Biosciences, CA, USA). To examine expression of intracellular markers, we surface-stained, fixed, permeabilized, and stained the cells for intracellular proteins using the Fixation and Permeabilization Buffer Kit as per the manufacturer's recommendations (Invitrogen, MA, USA). We used appropriate FMO controls as indicated. We analyzed the data using Flowjo (version 10, Tree Star).

### T Cell Sorting

We sorted bulk T cells from patient PBMC samples immediately after thawing on a BD FACS Aria flow cytometer (BD Biosciences, CA, USA). We sorted CD107a⁻ and CD107a⁺ CD8⁺ T cells after 7 days of peptide stimulation. We submitted the sorted T cell samples to TCR Vb sequencing, single cell RNA/TCR sequencing, or single cell TCR sequencing as indicated.

### Single Cell RNA/TCR Sequencing

Library preparations for single-cell immune profiling, sequencing, and post-processing of the raw data were performed at the Epigenomics Core at Weill Cornell Medicine.

### Sample Preparation

Single-cell RNA-seq libraries were prepared according to 10× Genomics specifications (Chromium Single Cell V(D)J User Guide PN-1000006, 10× Genomics, Pleasanton, CA, USA). Each cellular suspension (>90% viability) at a concentration between 700-1000 cells/μl, were loaded onto to the 10× Genomics Chromium platform to generate Gel Beads-in-Emulsion (GEM), targeting about 10000 single cells per sample. After GEM generation, polyA cDNA barcoded at the 5′end by the addition of a template switch oligo (TSO) linked to a cell barcode and Unique Molecular Identifiers (UMIs), was generated by an incubation at 53° C. for 45 min in a C1000 Touch Thermal cycler with 96-Deep Well Reaction Module (Bio-Rad, Hercules). GEMs were broken and the single strand cDNA was cleaned up with DynaBeads MyOne Silane Beads (Thermo Fisher Scientific, Waltham, MA). The cDNA was amplified for 13 cycles (98° C. for 45 sec; 98° C. for 20 s, 67° C. for 30 s, 72° C. for 1 h). Quality and quantity of the cDNA was assessed using an Agilent Bioanalyzer 2100 (Santa Clara, CA), obtaining a product of about 1600 bp. For generation of 5P expression libraries, an aliquot of the cDNA (about 50ng) was enzymatically fragmented, end repaired, A-tailed, subjected to a double-sided size selection with SPRI select beads (Beckman Coulter, IN, USA) and ligated to adaptors provided in the kit. A unique sample index for each library was introduced through 14 cycles of PCR amplification using the indexes provided in the kit (98° C. for 45 s; 98° C. for 20 s, 54° C. for 30 s, and 72° C. for 20 s×14 cycles; 72° C. for 1 min; held at 4° C.). Indexed libraries were subjected to a second double-sided size selection, and libraries were then quantified using Qubit fluorometric quantification (Thermo Fisher Scientific, Waltham, MA). The quality was assessed on an Agilent Bioanalyzer 2100, obtaining an average library size of 430 bp. For generation of full-length T-Cell-Receptor VDJ regions, an aliquot of the cDNA (about 5ng) was subjected to nested PCR amplification with specific VDJ outer and inner primer pairs (98° C. for 45 s; 98° C. for 20 s, 67° C. for 30 s, and 72° C. for 20 s x 8 cycles; 72° C. for 1 min; held at 4° C.), and one-sided size selection using SPRI select beads. Quality and quantity of the VDJ region was assessed using an Agilent Bioanalyzer 2100 (Santa Clara, CA). Average library size was 620 bp.

### Sequencing and Post-Processing of Data

5P expression and TCR libraries were clustered on an Illumina NovaSeq pair end read flow cell and sequenced for 28 cycles on R1 (10× barcode and the UMIs), followed by 8 cycles of 17 Index (sample Index), and 91 bases on R2 (transcript), obtaining about 250M clusters for 5P expression and 50M for TCR libraries. Primary processing of sequencing images was done using Illumina's Real Time Analysis software (RTA). 10× Genomics Cell Ranger Single Cell Software suite (support.10× genomics.com/single-cell-geneexpression/software/pipelines/latest/what-is-cell-ranger was used to perform sample) was used for demultiplexing, alignment (hg19), filtering, UMI counting, single-cell 5′end gene counting, TCR assembly, annotation of paired VDJ and performing quality control using the manufacturer parameters.

### Analysis

Filtered gene expression matrices generated from 10× CellRanger for five samples were matched to paired TCR sequences using the python package Scirpy¹⁷. All five samples were aggregated into a single unnormalized counts matrix and all downstream analysis was performed using GeneVector18. Batch effect correction was applied over all cells using the samples as batch labels. Cells were first classified as either CD4 or CD8 T cells using the respective gene marker. CD8+ T cells were further classified into four phenotypes (Effector, Memory, Naive, and Dysfunctional) using previously published gene markers^{19,20}. A probability distribution over phenotypes was generated for each cell and phenotype assignment corresponded to the maximum probability. Vaccine specific T cells were identified by exact match of the associated nucleotide sequence. UMAP visualizations were constructed using the python library Scanpy²¹.

### Clonality

Whole exome sequence reads of tumor-normal paired samples of patients were aligned to the reference human genome (hg19) using the Burrows-Wheeler Alignment tool (bwa mem v0.7.17) and samtools (v1.6). Duplicates were marked with picard-2.11.0 MarkDuplicates (broadinstitute.github.io/picard). Indel realignments were done with the Genome Analysis toolkit (GenomeAnalysisTK-3.8-1-0-gf15c1c3ef) RealignerTargetCreator and IndelRealigner22 using 1000 genome phase1 indel (1000G_phase1.indels.b37.vcf) and Mills indel calls (Mills_and_1000G_gold_standard.indels.b37.vcf) as references. Base calls were recalibrated with BaseRecalibrator and dbSNP version 138. Both tumor samples were covered at 378× and normal samples at 346× on average on its target regions. MuTect 1.1.7 and Strelka 1.0.15 were used to call SNVs and indels on pre-processed sequencing data. For the MuTect calls, dbSNP 138 and CosmicCodingMuts.vcf version 8623 were used as reference files. For the Strelka calls, we set “isSkipDepthFilters=1” to prevent filtering-out of mutation calls from exome sequencing due to exome-sequencing mapping breadth. Unbiased normal/tumor read counts for each SNV and indel call were then assigned with the bam-readcount software 0.8.0-unstable-6-963acab-dirty (commit 963acab-dirty)) (github.com/genome/bam-readcount). A minimum base quality filter was set with the “-b 15” flag. The reads were counted in an insertion-centric way with the “-i” flag, so that reads overlapping with insertions were not included in the per-base read counts. We then use the normal/tumor read counts to filter mutations. Filtering criteria are 1) total coverage for tumor ≥10, 2) variant allele frequency (VAF) for tumor ≥2%, 3) number of reads with alternative allele ≥5 for tumor, 4) total coverage for normal ≥7, and 5) VAF for normal ≤1% at a given mutation. Filtered mutation sets were annotated by SnpEff (v4.3t). In addition to point mutations, we called tumor somatic copy number variations in tumor using FACETS (github.com/mskcc/facets). Dbsnp138 (b37) was used for snp-pileup.

To infer clonality of vaccine targets, we extracted missense and frameshift mutations from the filtered VCF files and these mutations were put into PhyloWGS software package (github.com/morrislab/phylowgs) along with CNV calls for phylogeny reconstruction. Among 10,000 trees from PhyloWGS, we take top five trees based on the likelihood and compute average entropy level to measure tumor heterogeneity. For a given tree, we compute exclusive clone frequencies such as

$${x}^{a} = {x}^{a} - {\sum }_{β ∈ D ( α )} {x}^{β}$$

where D(a) is the set of clones that are direct descendants of clone w in the given tree and −u is cellular cancer fraction (CCF) of clone w. Based on exclusive clone frequencies, we computed Shannon's entropy as a measure of tumor heterogeneity as below:

$$S = {〈 - {\sum }_{α ∈ τ} {x}^{a} log ( {x}^{a} 〉}_{τ}$$

where [symbol] _{τ} is the arithmetic average operator from top 5 trees (τ).

[symbol]

### Immunofluorescence

Automated double immunofluorescence was conducted using the Leica Bond BX staining system. Paraffin embedded tissues were sectioned at 5 μm and baked at 58° C. for 1 h. Slides were loaded in Leica Bond and staining was performed as follows. Samples were pretreated with EDTA-based epitope retrieval ER2 solution (Leica, AR9640) for 20 min at 95° C. The double antibody staining and detection were conducted sequentially. Primary antibodies against CD3 (0.6 μg/ml, Rabbit, Dako #A0452) and CD8 (1/10, Rabbit, Ventana (Roche) #790-4460) were used. The Leica Bond Polymer anti-rabbit HRP secondary antibody was applied followed by Alexa Fluor tyramide signal amplification reagents (Life Technologies, B40953) or CFR dye tyramide conjugates (Biotium, 92174) for detection. After CD3 staining, epitope retrieval was performed for denaturation of primary and secondary antibodies before CD8 antibody was applied. After the run was finished, slides were washed in PBS and incubated in 5 μg/ml 4′,6-diamidino-2-phenylindole (DAPI) (Sigma Aldrich) in PBS for 5 min, rinsed in PBS, and mounted in Mowiol 4-88 (Calbiochem). Slides were kept overnight at −20° C. before imaging. Slides were scanned on a Pannoramic Scanner (3DHistech, Budapest, Hungary) using a 20×/0.8NA objective. Whole tissues were annotated in CaseViewer (3DHistech) and converted to Tiff images. ImageJ was used to segment cells based on DAPI, and to quantify whether a given cell is single, double, or null positive.

Digital Droplet PCR (ddPCR)

DNA extraction and detection of TP53 R175H mutation by ddPCR were performed by the Integrated Genomics Operation Core at MSKCC.

### DNA Extraction

FFPE curls collected in AutoLys M tubes (Thermo Fisher catalog #A38738) were digested with Protease Solution. DNA was extracted using the MagMAX FFPE DNA/RNA Ultra Kit (Thermo Fisher catalog #A31881) on the KingFisher Flex Purification System (Thermo Fisher) according to the manufacturer's protocol. Samples were eluted in 55 μl elution solution.

### Detection of TP53 R175H Mutation by Digital Droplet PCR

TP53 assays were ordered through Bio-Rad (assay IDs: dHsaCP2000105—TP53 p.R175H c.524G>A; dHsaCP2000106—TP53 WT). Cycling conditions were tested to ensure optimal annealing/extension temperature as well as optimal separation of positive from empty droplets. Optimization was done with a known positive control.

After PicoGreen quantification, 9 ng gDNA were combined with locus-specific primers, FAM- and HEX-labeled probes, HaeIIL, and digital PCR Supermix for probes (no dUTP). All reactions were performed on a QX200 ddPCR system (Bio-Rad catalog #1864001) and each sample was evaluated in technical duplicates. Reactions were partitioned into a median of ˜22,000 droplets per well using the QX200 droplet generator. Emulsified PCRs were run on a 96-well thermal cycler using cycling conditions identified during the optimization step (95° C. 10′; 40 cycles of 94° C. 30′ and 55° C. 1′; 98° C. 10′; 4° C. hold). Plates were read and analyzed with QuantaSoft software to assess the number of droplets positive for mutant DNA, wild-type DNA, both, or neither.

### REFERENCES CITED IN THIS EXAMPLE

1. Kranz, L. M. et al. Systemic RNA delivery to dendritic cells exploits antiviral defence for cancer immunotherapy. Nature 534, 396-401 (2016).
2. Robins, H. S. et al. Digital Genomic Quantification of Tumor-Infiltrating Lymphocytes. Science Translational Medicine 5, 214ra169-214ra169 (2013).
3. Carlson, C. S. et al. Using synthetic templates to design an unbiased multiplex PCR assay. Nat Commun 4, 2680 (2013).
4. Robins, H. et al. Ultra-sensitive detection of rare T cell clones. J Immunol Methods 375, 14-19 (2012).
5. Sethna, Z., Elhanati, Y., Callan, C. G., Walczak, A. M. & Mora, T. OLGA: fast computation of generation probabilities of B- and T-cell receptor amino acid sequences and motifs. Bioinformatics 35, btz035— (2019).
6. Sahin, U. et al. BNT162b2 vaccine induces neutralizing antibodies and poly-specific T cells in humans. Nature 595, 572-577 (2021).
7. Moodie, Z., Huang, Y., Gu, L., Hural, J. & Self, S. G. Statistical positivity criteria for the analysis of ELISpot assay data in HIV-1 vaccine trials. J Immunol Methods 315, 121-32 (2006).

8. Moodie, Z. et al. Response definition criteria for ELISPOT assays revisited. Cancer Immunol Immunother 59, 1489-1501 (2010).
9. Balachandran, V. P. et al. Identification of unique neoantigen qualities in long-term survivors of pancreatic cancer. Nature 551, 512-516 (2017).
10. Luksza, M. et al. Neoantigen quality predicts immunoediting in survivors of pancreatic cancer. Nature 606, 389-395 (2022).
11. Gallardo, H. F., Tan, C., Ory, D. & Sadelain, M. Recombinant Retroviruses Pseudotyped With the Vesicular Stomatitis Virus G Glycoprotein Mediate Both Stable Gene Transfer and Pseudotransduction in Human Peripheral Blood Lymphocytes. Blood 90, 952-957 (1997).
12. Ghani, K. et al. Efficient Human Hematopoietic Cell Transduction Using RD114- and GALV-Pseudotyped Retroviral Vectors Produced in Suspension and Serum-Free Media. Hum Gene Ther 20, 966-974 (2009).
13. Balachandran, V. P. et al. Identification of unique neoantigen qualities in long-term survivors of pancreatic cancer. Nature 551, 512-516 (2017).
14. Riviere, I., Brose, K. & Mulligan, R. C. Effects of retroviral vector design on expression of human adenosine deaminase in murine bone marrow transplant recipients engrafted with genetically modified cells. Proc National Acad Sci 92, 6733-6737 (1995).
15. Gros, A. et al. Prospective identification of neoantigen-specific lymphocytes in the peripheral blood of melanoma patients. Nat Med 22, 433-438 (2016).
16. Cohen, C. J., Zhao, Y., Zheng, Z., Rosenberg, S. A. & Morgan, R. A.

Enhanced Antitumor Activity of Murine-Human Hybrid T-Cell Receptor (TCR) in Human Lymphocytes Is Associated with Improved Pairing and TCR/CD3 Stability. Cancer Res 66, 8878-8886 (2006).
17. Sturm, G. et al. Scirpy: A Scanpy extension for analyzing single-cell T-cell receptor sequencing data. Bioinformatics 36, btaa611—(2020).
18. Ceglia, N. et al. GeneVector: Identification of transcriptional programs using dense vector representations defined by mutual information. Biorxiv 2022.04.22.487554 (2022) doi:10.1101/2022.04.22.487554.
19. Akondy, R. S. et al. Origin and differentiation of human memory CD8 T cells after vaccination. Nature 552, 362-367 (2017).
20. Leun, A. M. van der, Thommen, D. S. & Schumacher, T. N. CD8+ T cell states in human cancer: insights from single-cell analysis. Nat Rev Cancer 20, 218-232 (2020).
21. Wolf, F. A., Angerer, P. & Theis, F. J. SCANPY: large-scale single-cell gene expression data analysis. Genome Biol 19, 15 (2018).
22. McKenna, A. et al. The Genome Analysis Toolkit: A MapReduce framework for analyzing next-generation DNA sequencing data. Genome Res 20, 1297-1303 (2010).
23. Tate, J. G. et al. COSMIC: the Catalogue Of Somatic Mutations In Cancer. Nucleic Acids Res 47, gky1015-(2018).

### Example 2: Statistical Method for Identifying T Cell Clones Responsive to Immunotherapy

The statistical method described herein was designed to determine whether two-fold expansion between a baseline and comparative distribution of T cell receptor sequence counts in a sample is significant in a manner less sensitive to particulars of the entire distribution, where the number of T cells with a given T cell receptor sequence can vary over many orders of magnitude. To determine which clones in a comparative sample are significantly 2-fold expanded, we utilized a modified form of the Fisher exact test. A clonotype can refer to T cells with the same T cell receptor CDR3 nucleotide sequence within a sample. The baseline sample size is rescaled to assign significance to a two-fold clonal expansion (that is the total number of T cells sequenced in the baseline sample, N, is rescaled to N/2), to compare to the comparative sample with M cells sequenced. This leads to a categorical table where in Category 1 the number of T cells with clonotype, x, in the baseline sample is nₓ, and in the comparative sample the number of cells with the same clonotypes is mₓ. In Category 2 are the number of T cells not having clonotype, x. There are N/2-nₓ and M-mₓ members of Category 2 in the rescaled baseline and comparative sample respectively. P-values are then determined by Fisher's exact test with a Bonferroni correction for the total number of p values generated. That p-value for whether clonotype x had a significantly expanded number of clonotypes in the comparative sample, pₓ, is multiplied by the total number of unique clonotypes in either the baseline or comparative sample, |NUM|. This new statistical method allows for precise determination of clones that expand between two given samples by accurately distinguishing clones that expand from clones that otherwise vary stochastically.

### Example 3: Immune Activity. Feasibility. And Safety of Individualized mRNA Neoantigen Vaccines in Pancreatic Ductal Adenocarcinoma

### Abstract

Pancreatic ductal adenocarcinoma (PDAC) is lethal in 90% of patients. However, recent discoveries have revealed PDACs harbor mutation-derived neoantigens suited for vaccines.

We conducted an investigator-initiated, phase-I trial to use adjuvant autogene cevumeran, a per-patient mRNA neoantigen vaccine, to activate T cells in PDAC. From surgically resected PDACs, we custom synthesized mRNA neoantigen vaccines in real-time. After surgery, we sequentially administered atezolizumab (PDL-1 inhibitory antibody), autogene cevumeran, and modified (m) FOLFIRINOX. The primary endpoint was safety. Other endpoints included immune response, 18-month recurrence-free survival (RFS), and feasibility of individualized cancer vaccination.

We treated 16 patients with atezolizumab, and autogene cevumeran, and 15 with mFOLFIRINOX. Autogene cevumeran induced substantial immune responses in 8 of 16 patients, expanding circulating T cells from undetectable levels to up to 10% of all blood T cells. Vaccine-expanded T cells included polyfunctional neoantigen-specific effector CD8⁺ T cells expressing lytic granules. Vaccine-expanded T cells durably persisted despite mFOLFIRINOX at up to 2.5% of all blood T cells even 2 years after surgery. At a median follow-up of 18 months, immune responders with vaccine-induced T cells had a median RFS that was not reached (vs. 13.4 months in immune non-responders; P=0.003). Individualized vaccines were administered within 3 days of benchmarked times. One patient had grade 3 fever and hypertension.

Adjuvant atezolizumab, autogene cevumeran, and mFOLFIRINOX provokes substantial and durable T cell activity that correlates with delayed PDAC recurrence.

### Introduction

Pancreas ductal adenocarcinoma (PDAC) is the third leading cause of cancer death in the United States,¹ and the seventh leading cause of cancer death worldwide.² Though mortality has decreased for nearly all other common cancers, survival rates for PDAC have stagnated for over 60 years.³ Five-year overall survival (OS) for patients with PDAC remains dismal at <10%.³ Multi-agent chemotherapy is the standard of care for the 85% of patients who present with distant metastases or surgically unresectable tumors, but confers a median survival of only <18 months.⁴⁻⁶ Surgery and adjuvant combination chemotherapy is the standard in the 15% of patients with surgically resectable tumors. However, nearly 80% of these patients recur at ˜14 months, and their 5-year OS is only <300.⁷ Radiation, biologic, and targeted therapies are also ineffective.¹

PDACs are also near completely insensitive to immunotherapies, with a <5%^{8,9} response rate (RR) to immune checkpoint inhibitors. This low RR is partially attributed to PDACs harboring a low mutation rate that generates few neoantigens⁹—mutation-generated proteins absent from normal tissues that can identify cancers as foreign to T cells—possibly rendering PDACs weakly antigenic, with fewer infiltrating T cells. However, recent studies have observed that most PDACs in fact harbor more neoantigens¹⁰⁻¹² than previously predicted.¹³ Furthermore, studies of rare long-term survivors of PDAC^{11,12} have revealed that neoantigens can stimulate T cells in PDAC, as primary tumors enriched in immunogenic neoantigens also harbor ˜12-fold higher densities of activated T cells, exhibit delayed recurrence, and longer patient survival. Thus, as most PDACs harbor neoantigens that can stimulate T cells, strategies to deliver neoantigens can induce neoantigen-specific T cells and impact PDAC outcomes.

Based on observations in long-term PDAC survivors,^{11,12} we validated whether adjuvant individualized vaccines can stimulate neoantigen-specific T cells and provide similar clinical benefit in surgically resected PDAC patients. We sought a platform to rapidly deliver individualized neoantigen vaccines fully integrated into a routine oncologic workflow. Therapeutic messenger RNA (mRNA) vaccine technology can enable such integration.¹⁴ As mRNA can be rapidly manufactured as individualized vaccines with multiple neoantigens,¹⁵ and can activate antigen presenting cells,¹⁶⁻²⁰ without wishing to be bound by theory, an effective individualized mRNA vaccine would elicit neoantigen-specific T cells in PDACs, eliminate micrometastases, and delay recurrence.

### Trial Design, Rationale, Treatments, Oversight, and Conduct

We conducted an investigator-initiated, phase-I clinical trial of sequential adjuvant atezolizumab (αPD-L1, Genentech, USA), autogene cevumeran^{21,22} (an individualized mRNA neoantigen vaccine; Individualized NeoAntigen Specific Therapy [iNeST], BioNTech, Germany and Genentech), and modified FOLFIRINOX (mFOLFIRINOX) in patients with surgically resectable PDAC to: (1) amplify neoantigen-specific T cells that are inhibited by PD-1 signaling, and (2) prime naïve T cells to vaccine-delivered neoantigens.

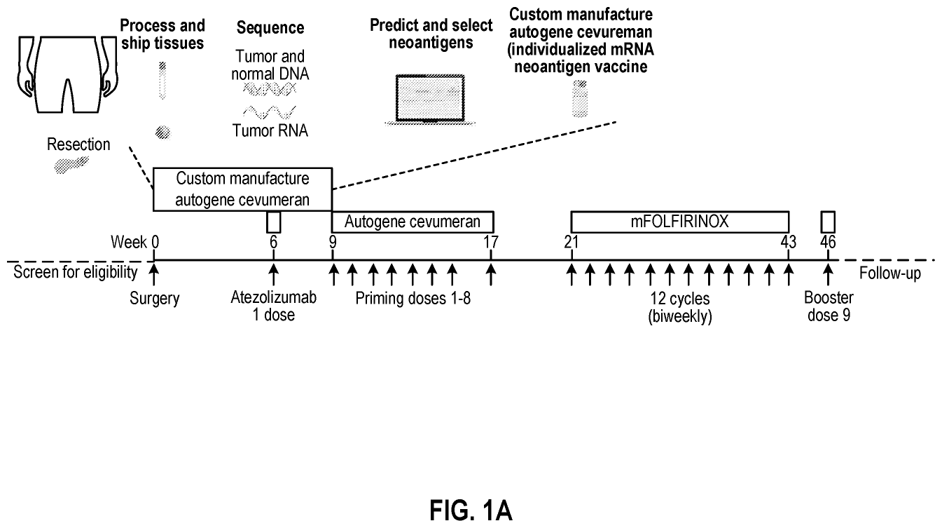

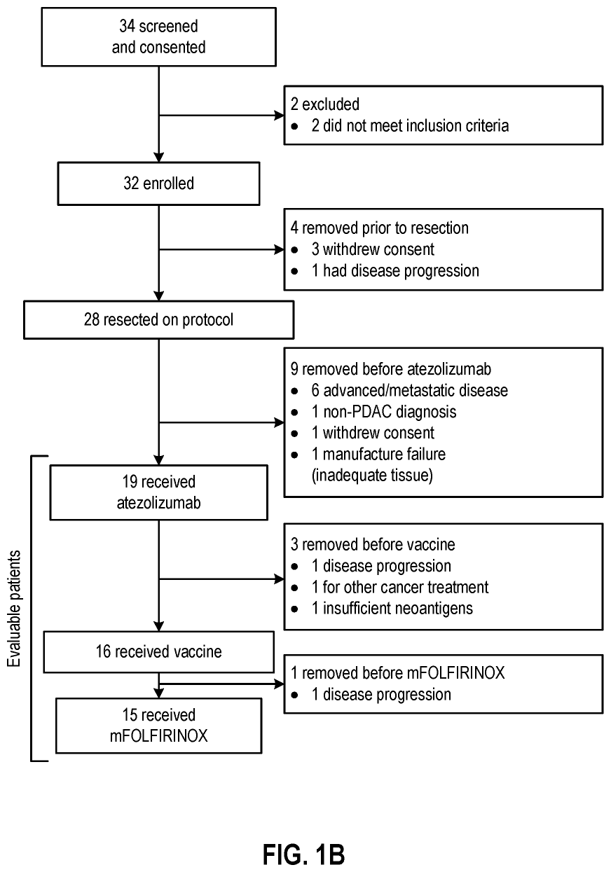

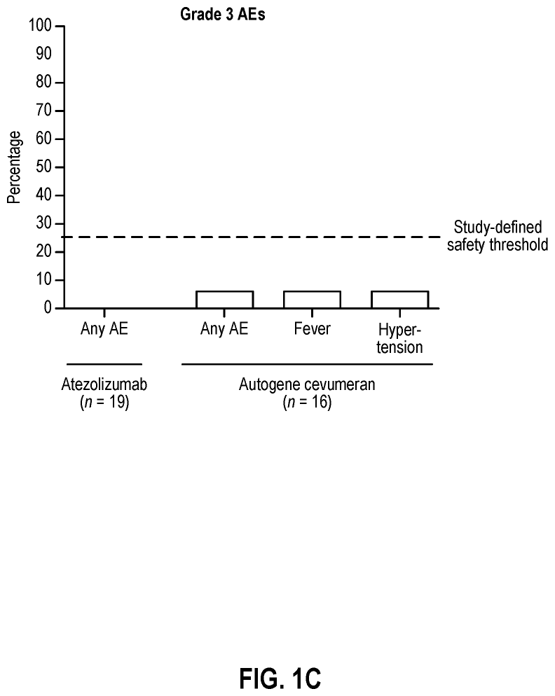

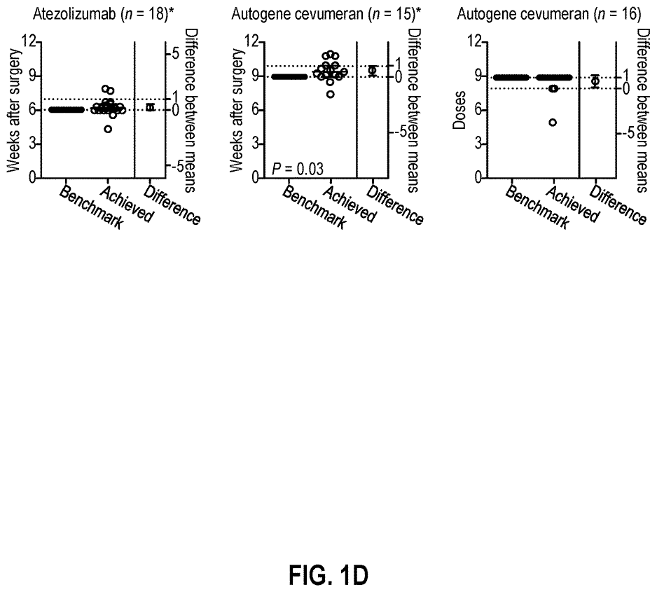

We dosed atezolizumab, autogene cevumeran, and mFOLFIRINOX sequentially to measure how each drug modulated neoantigen-specific T cells and set the following benchmarked times to treatment after surgery (FIG. 1A): (1) one 1200-mg IV dose of atezolizumab on week 6; (2) nine 25-μg IV doses of autogene cevumeran given as 7 weekly priming doses beginning on week 9, an 8^{th} dose at week 17, and a 9^{th} booster dose at week 46; (3) 12 cycles of mFOLFIRINOX beginning on week 21.

We enrolled patients of ECOG performance status 0-1 with single, radiographically suspicious, surgically resectable PDACs, no distant metastases, and ≥5 neoantigens. We excluded patients with metastatic, borderline, or locally unresectable PDACs, and patients who received neoadjuvant therapy.

After surgery, we included patients with pathologically confirmed PDAC with R0/R1 margins, and excluded patients with non-PDAC tumors, unresolved Clavien-Dindo grade ≥3 postoperative complications,²³ and patients needing concurrent treatment for other malignancies. Additional eligibility criteria and ethical study conduct information are in the protocol. We targeted to accrue 20 evaluable patients.

### Surgery

Patients underwent open pancreaticoduodenectomy, or either open or laparoscopic distal pancreatectomy and splenectomy. We then shipped tumor blocks with the most (minimum >10%) histologic tumor content with matched blood to BioNTech (Mainz, Germany).

### Mutation Identification and Neoantigen Selection

DNA was extracted from normal peripheral blood mononuclear cells. DNA and RNA were extracted from tumors. Expressed non-synonymous mutations and HLA type were identified by whole-exome sequencing of patient-specific tumor/normal pairs and tumor RNA-sequencing. Neoantigens were bioinformatically predicted and ranked by immunogenicity using the autogene cevumeran Genentech Recurrent Attention Framework (GRAF) deep learning model.

### Autogene Cevumeran Manufacturing

For every patient, individualized mRNA neoantigen vaccines were manufactured under GMP conditions containing 2 mRNA strands, each strand encoding up to 10 MHC-I and MHC-II neoepitopes, formulated in ˜400 nm diameter lipoplex nanoparticles²⁴ to enable IV delivery.

### Endpoints

The primary endpoint was safety (Table 1). Secondary endpoints were 18-month recurrence-free survival (RFS) and 18-month OS. We defined recurrence as new lesions by RECIST 1.1, and RFS from either the date of surgery (RFS), or from the date of the last autogene cevumeran priming dose (landmark RFS) to the date of recurrence or death, whichever occurred earliest. We censored patients without events at the last known date they were recurrence-free. We defined OS from the date of surgery to the date of death. As exploratory endpoints, we measured immune response and feasibility as actual vs. benchmarked treatment times. Data cut-off was Apr. 1, 2022, extending the median follow-up beyond the prespecified 18-month RFS secondary endpoint.

TABLE 1
Primary Endpoint.

| Number of | Number of grade 3 or higher adverse events (AEs) due |
|---|---|
| patients | to atezolizumab and autogene cevumeran to stop trial |
| 3-10 | ≥3 |
| 11-16 | ≥4 |
| 17-20 | ≥5 |

### Immune Response

### Atezolizumab:

If atezolizumab expanded peripheral blood T cell clones (measured by TCR VD sequencing), we classified a patient as an atezolizumab responder.

### Autogene Cevumeran:

If autogene cevumeran expanded peripheral blood T cell clones that recognized neoantigens in patient's individualized mRNA vaccines by two independent assays (Assay 1:≥2-fold post-vaccination T cell clonal expansion by TCR Vβ sequencing; Assay 2: T cell IFNγ production by ex vivo ELISPot), we classified a patient as an autogene cevumeran responder. We adjudicated each assay as positive or negative blinded to all other assay results and endpoints.

### Statistical Analyses

Safety endpoints are presented descriptively as percentages. Sample sizes (n) represent the number of patients, tumors, T cell clones, or neoantigens. We analyzed feasibility as the statistical equivalence between benchmarked and achieved treatment times. Here, we define a delay of <1 week as the zone of clinical indifference, and define the achieved time to be statistically equivalent to the benchmarked time if the 90% confidence interval of the achieved time was within the zone of clinical indifference. We analyzed survival curves by log rank (Mantel Cox) test, and compared two groups using unpaired two-tailed Mann-Whitney test. P<0.05 was considered statistically significant. All analyses were two-tailed and performed using GraphPad Prism (version 9.3.1).

### Results

### Patients and Treatments

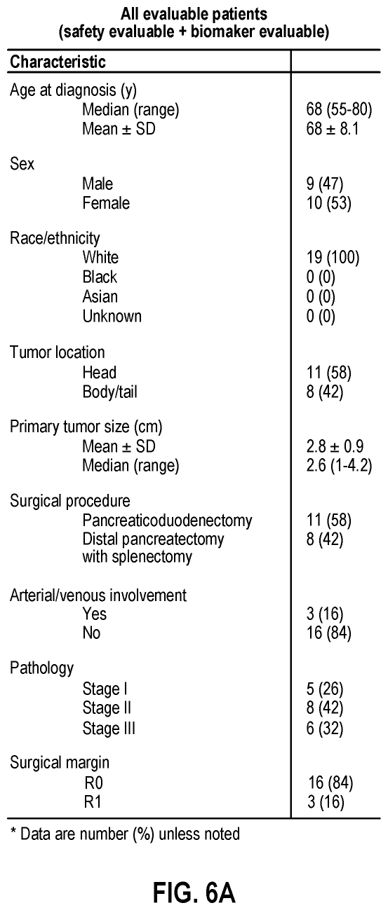

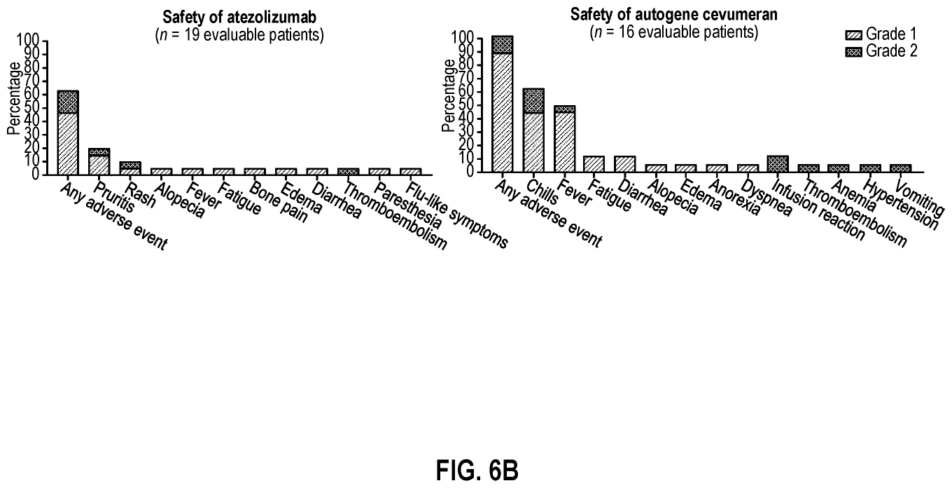

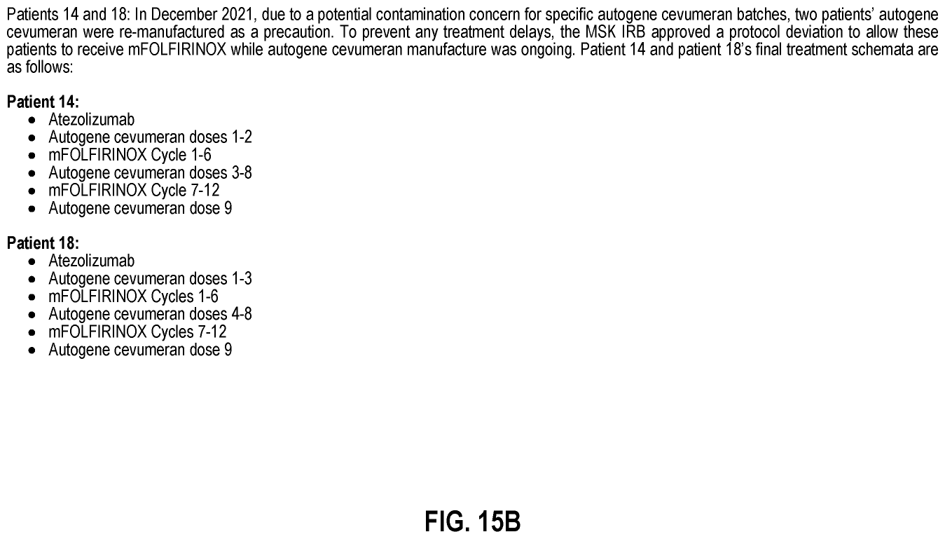

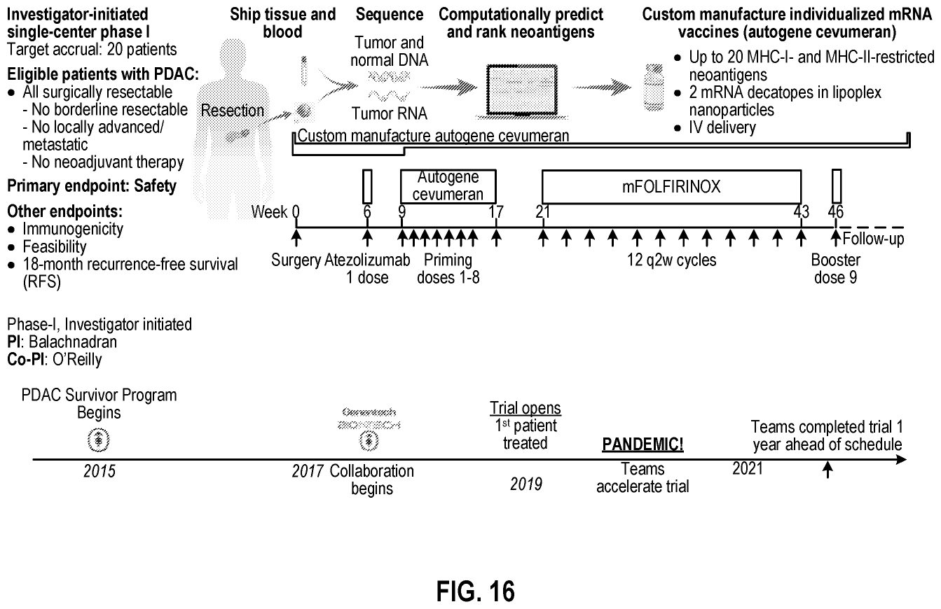

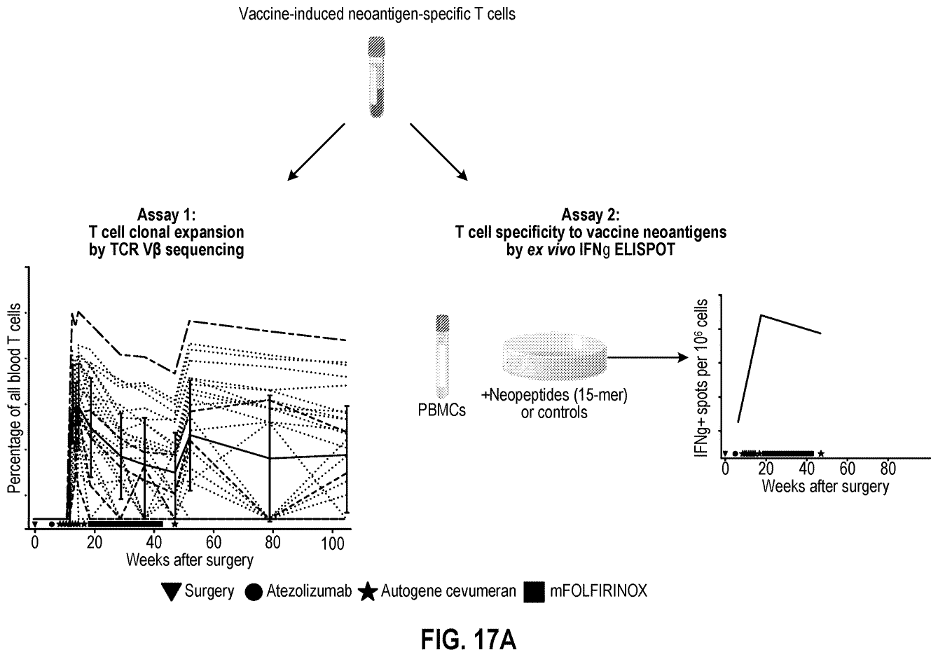

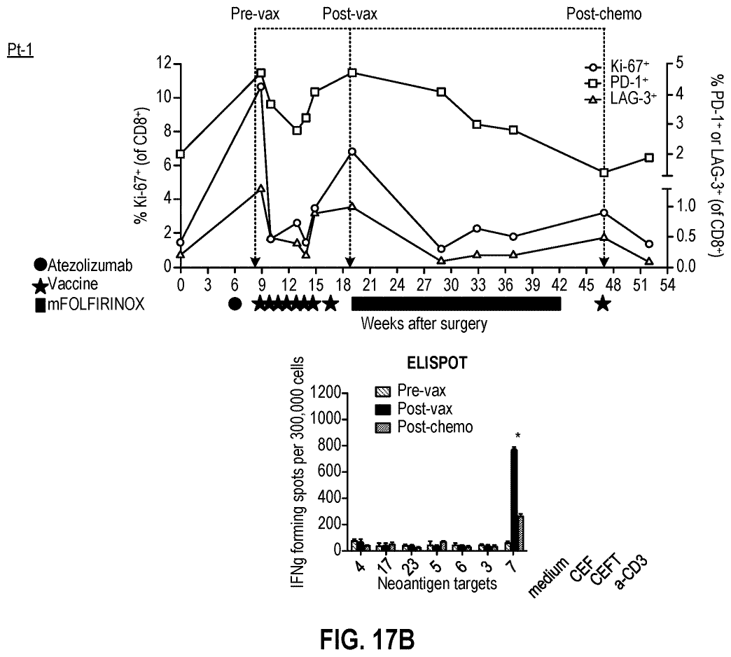

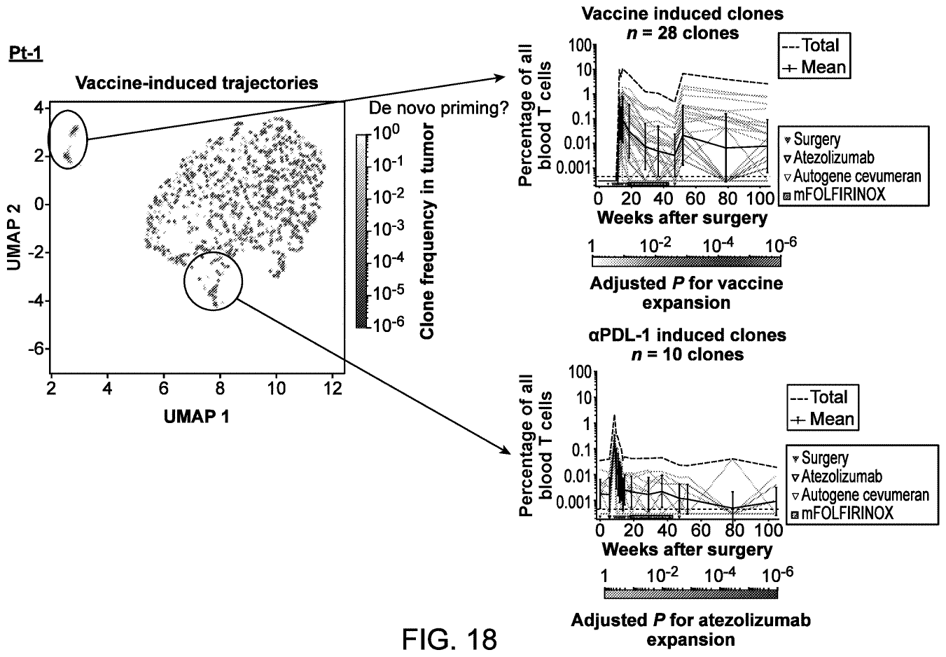

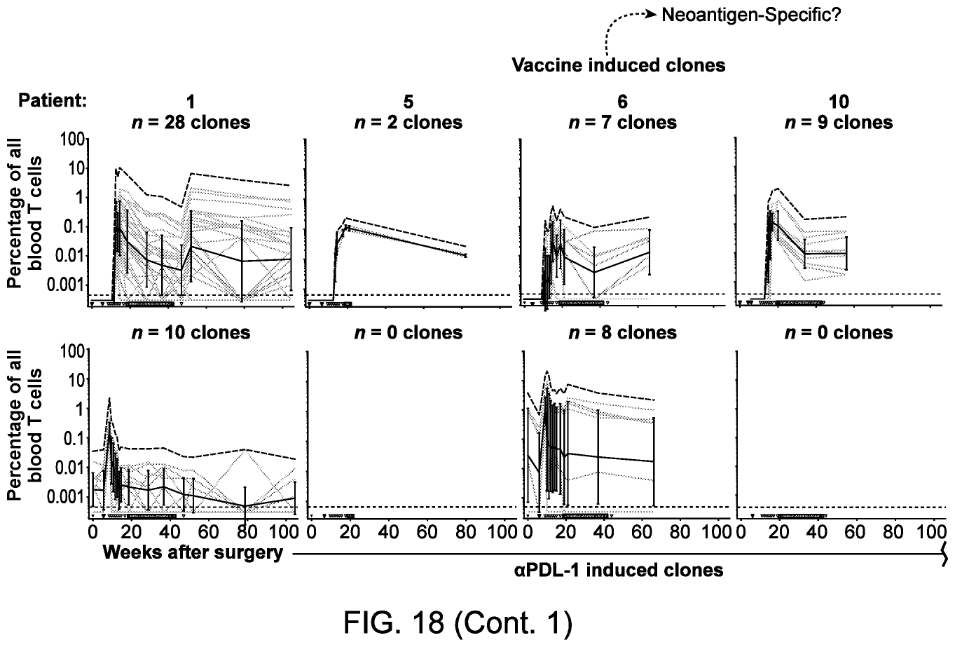

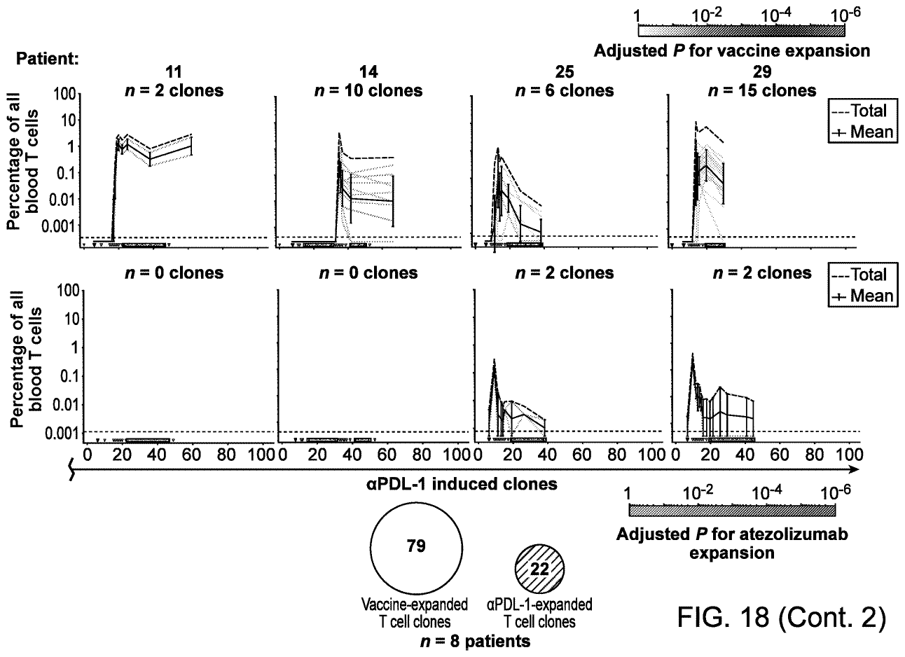

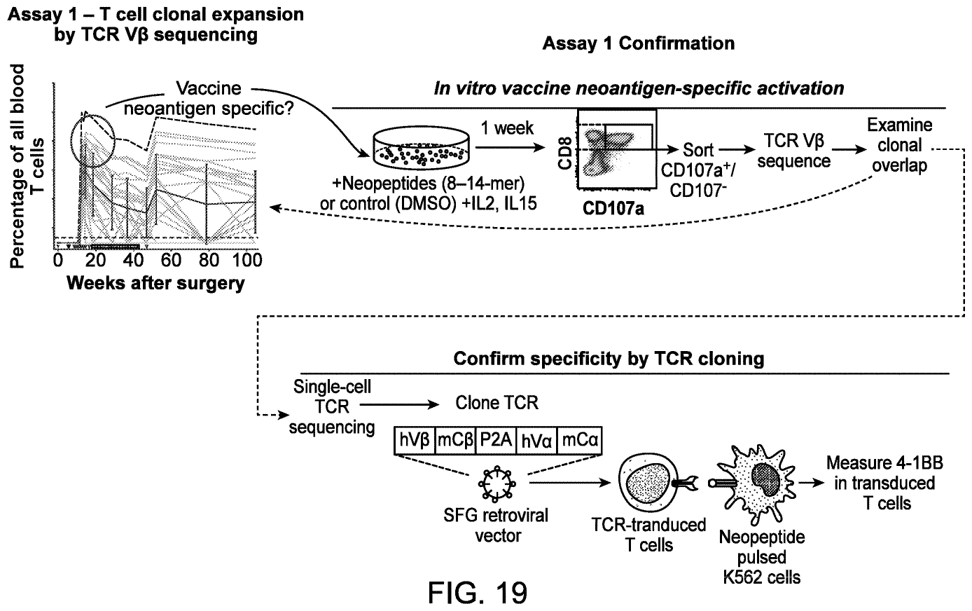

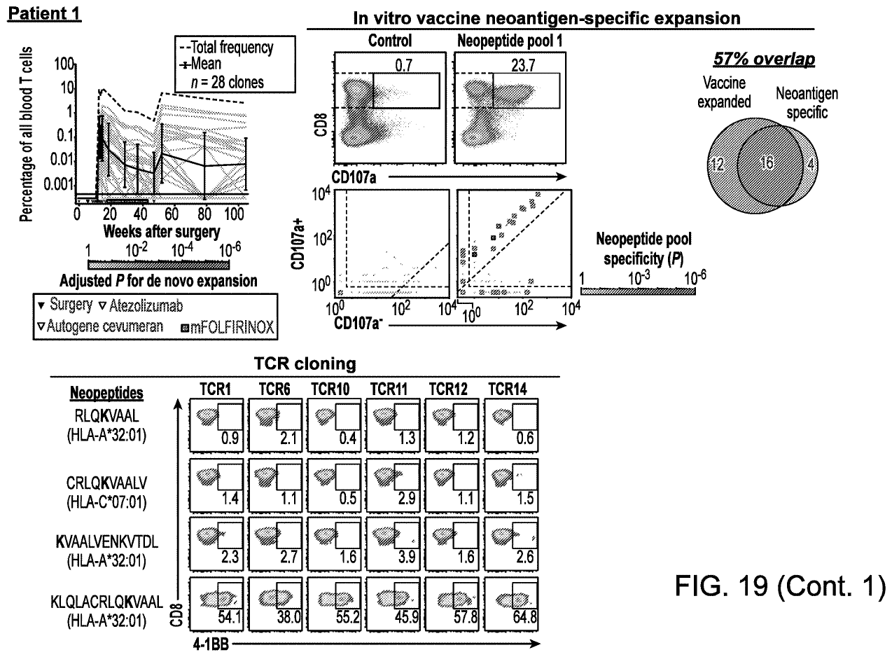

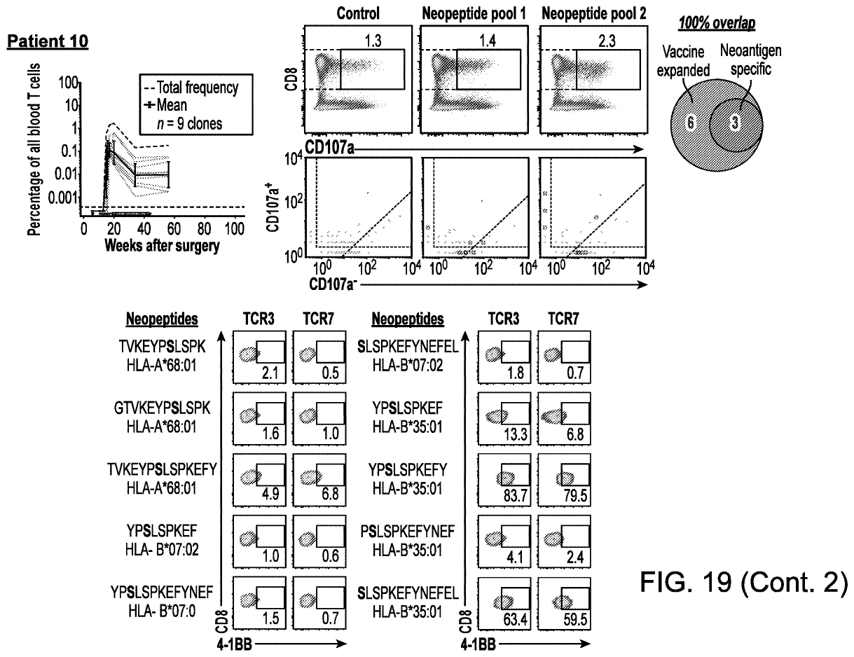

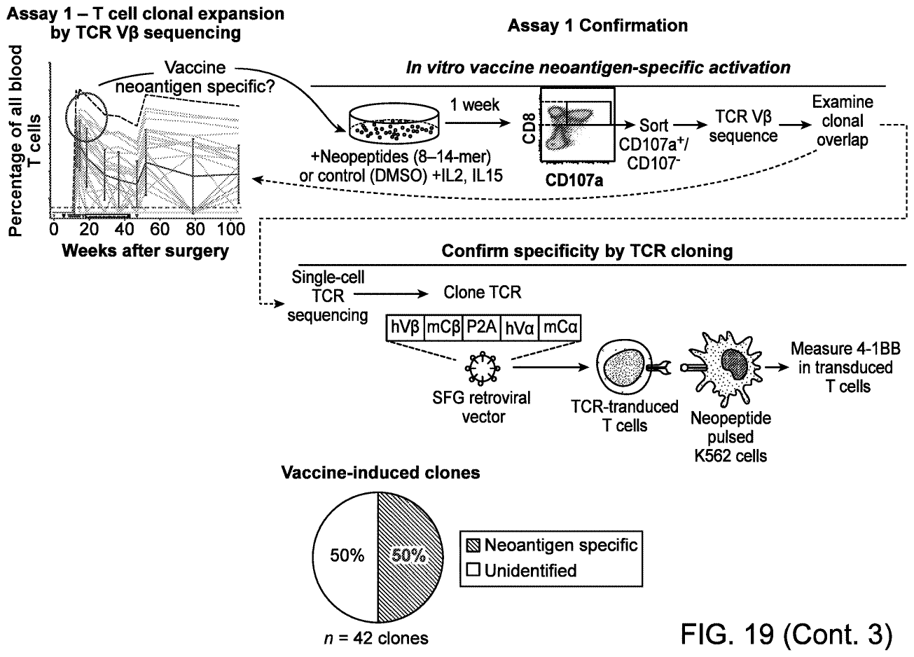

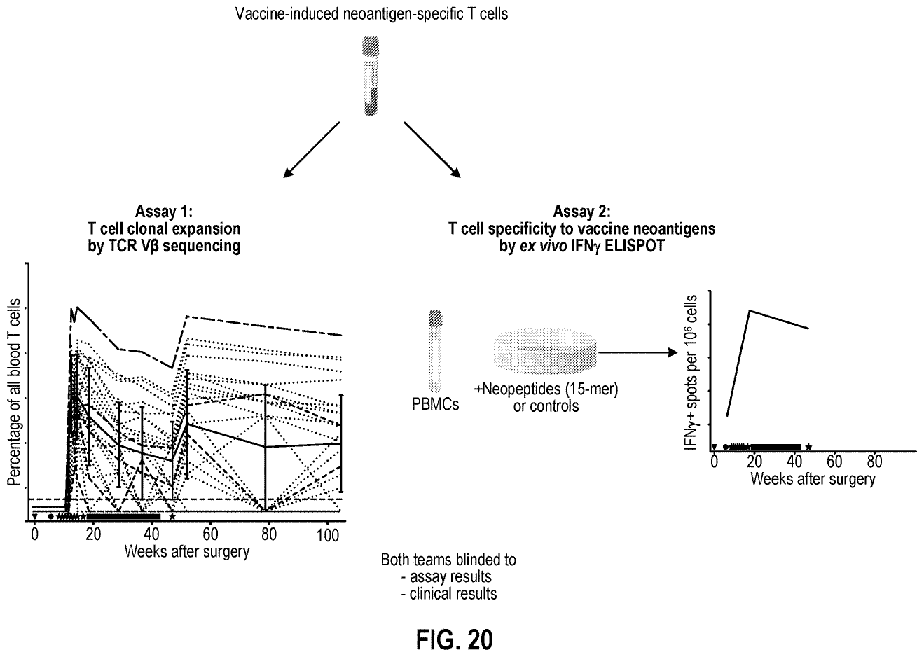

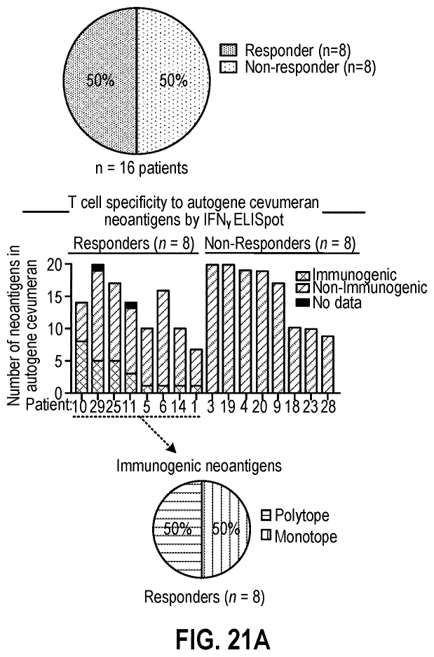

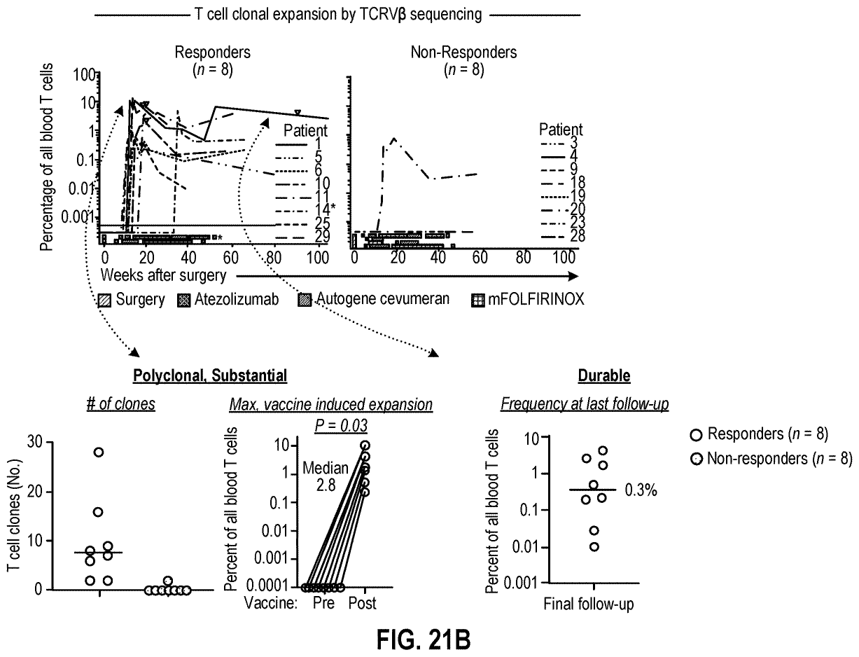

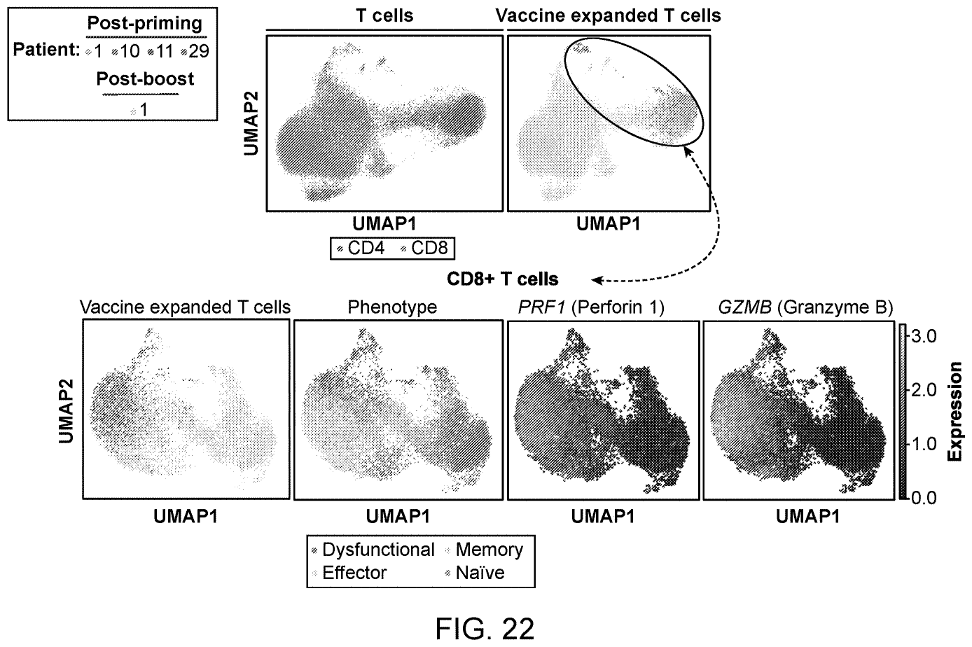

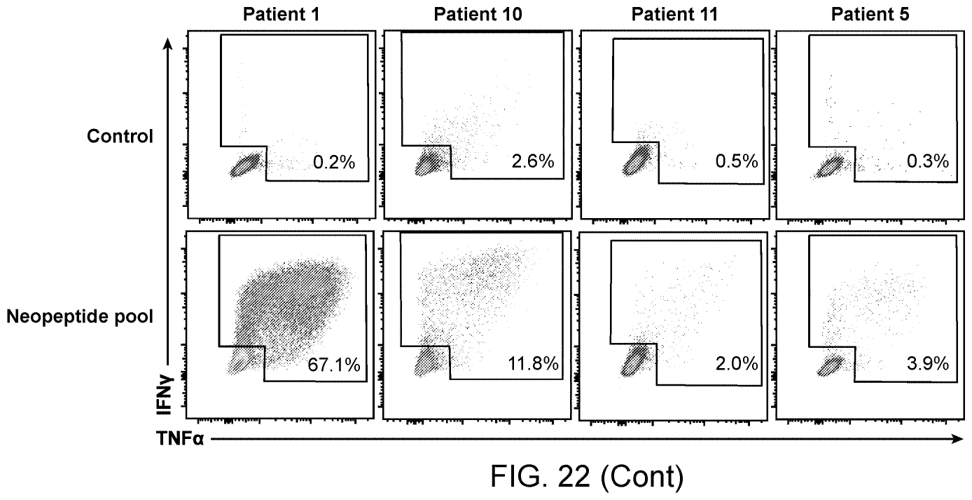

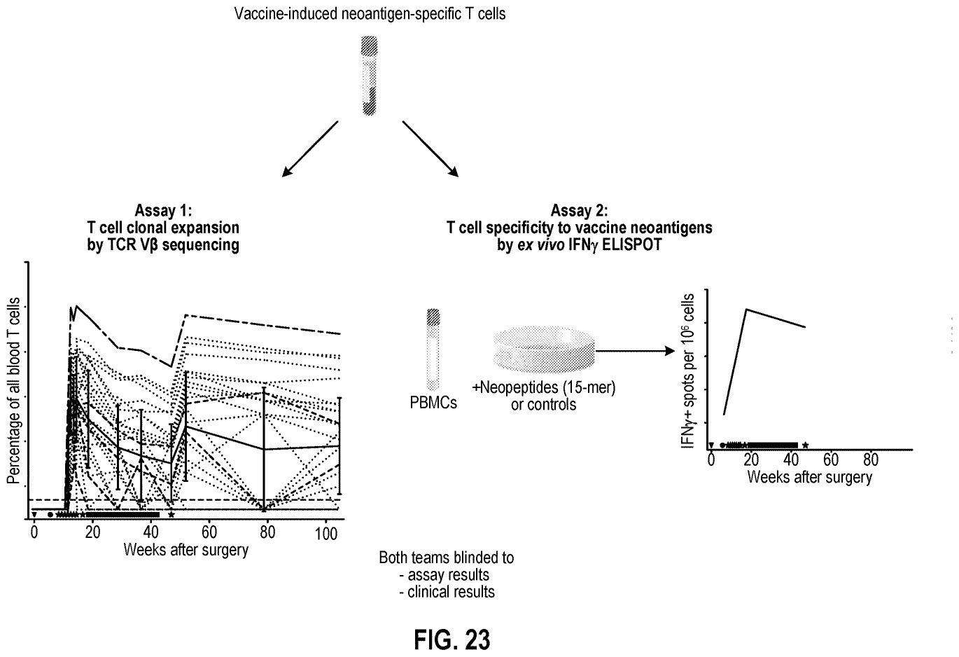

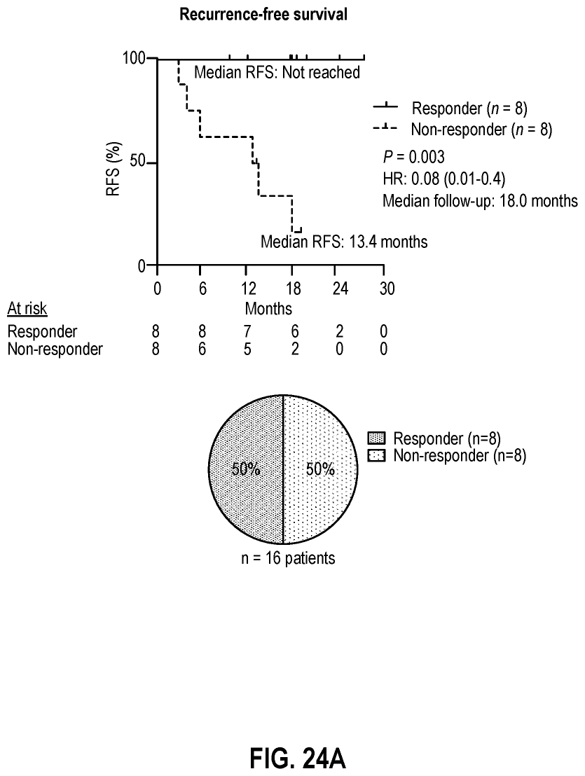

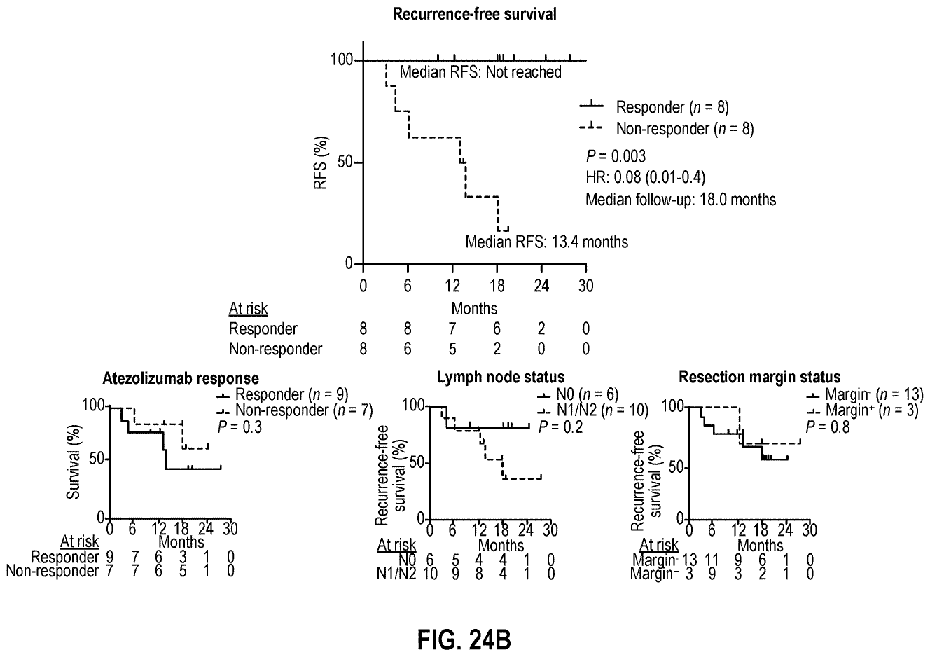

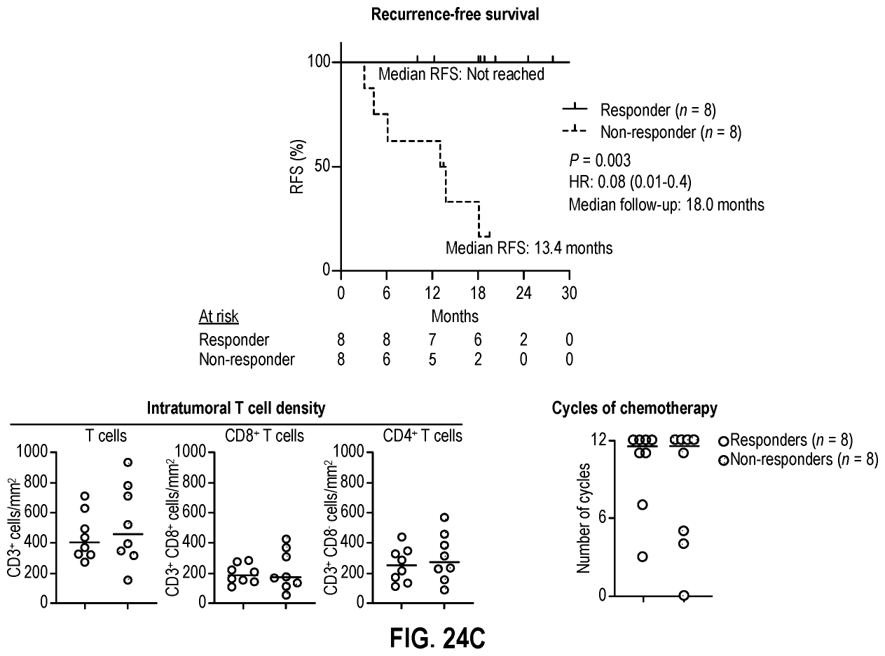

From December 2019 to August 2021, we enrolled 34 patients, of which 28 patients (FIG. 1B) underwent surgery. We then treated 19 patients with atezolizumab, 16 patients with autogene cevumeran, and 15 patients with mFOLFIRINOX (FIG. 1B). We analyzed safety in safety-evaluable cohorts (19 treated with atezolizumab, 16 with autogene cevumeran), and correlated immune response to RFS in a biomarker-evaluable cohort (16 treated with atezolizumab and autogene cevumeran). The 19 patients included in all evaluable cohorts had typical clinical characteristics (FIG. 6A; FIG. 15).

### Safety and Feasibility

None of the 19 atezolizumab safety-evaluable patients had grade 3 or higher AEs (FIG. 1C). Only one of 16 autogene cevumeran safety-evaluable patients (6%) had grade 3 AEs (fever and hypertension, FIG. 1C. All 16 patients (100%) had grade 1 and 2 AEs (FIG. 6B).

We administered atezolizumab and autogene cevumeran at median times within 1 and 3 days of respective benchmarked times (FIG. 1D). Only 1 of 19 (5%) patients had vaccine non-manufacture due to insufficient neoantigens (FIG. 1B). Three of 16 patients (19%) did not receive all 9 vaccine doses (FIG. 1D), due to progression, death, and mFOLFIRINOX toxicity.

### Vaccine Response

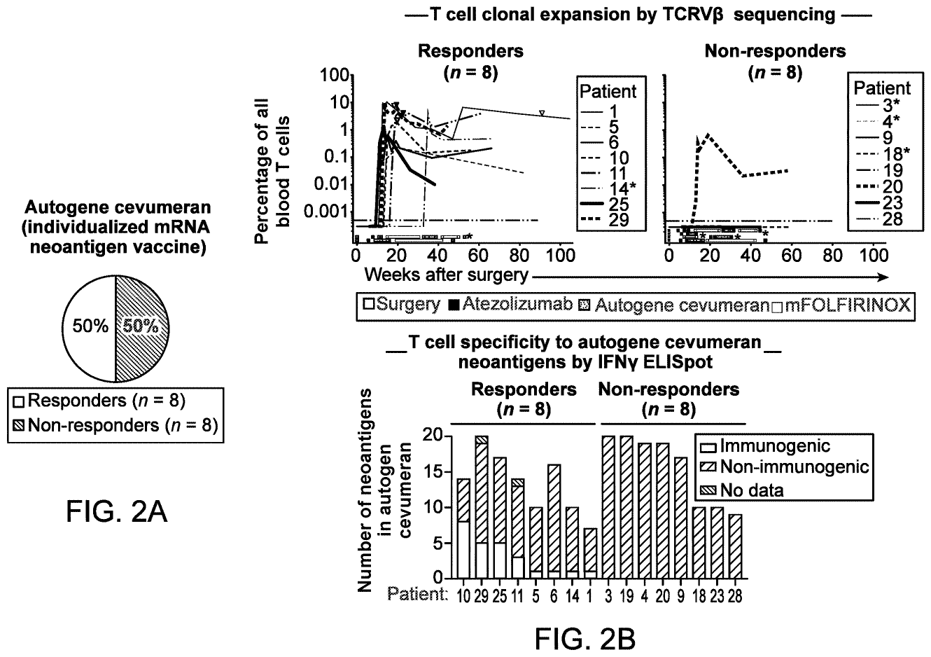

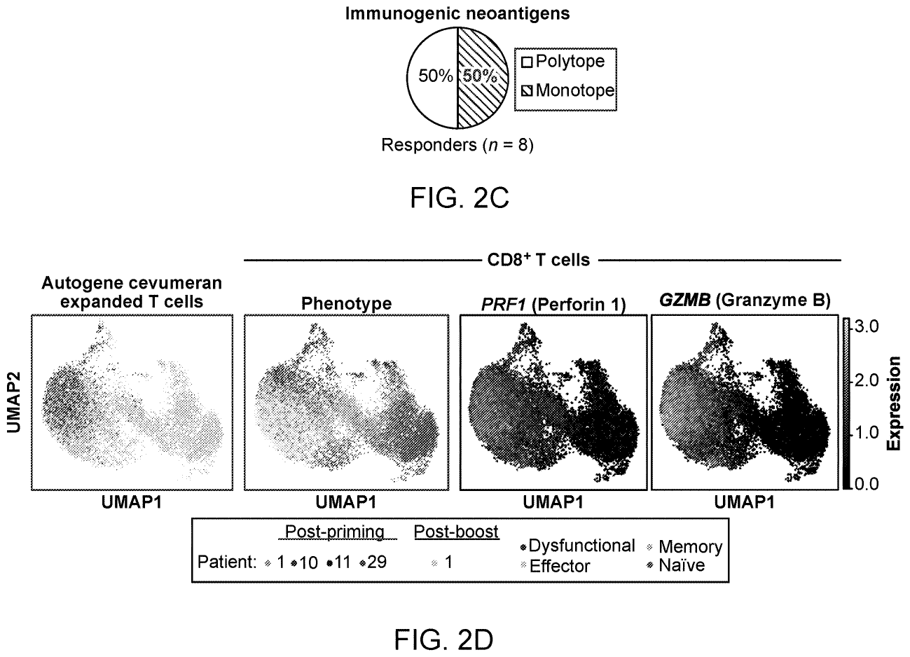

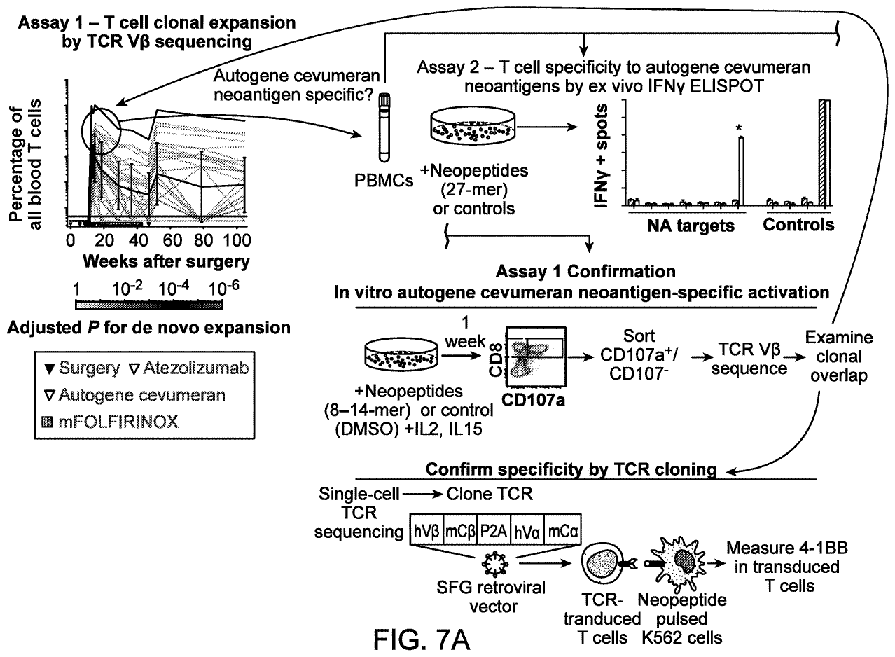

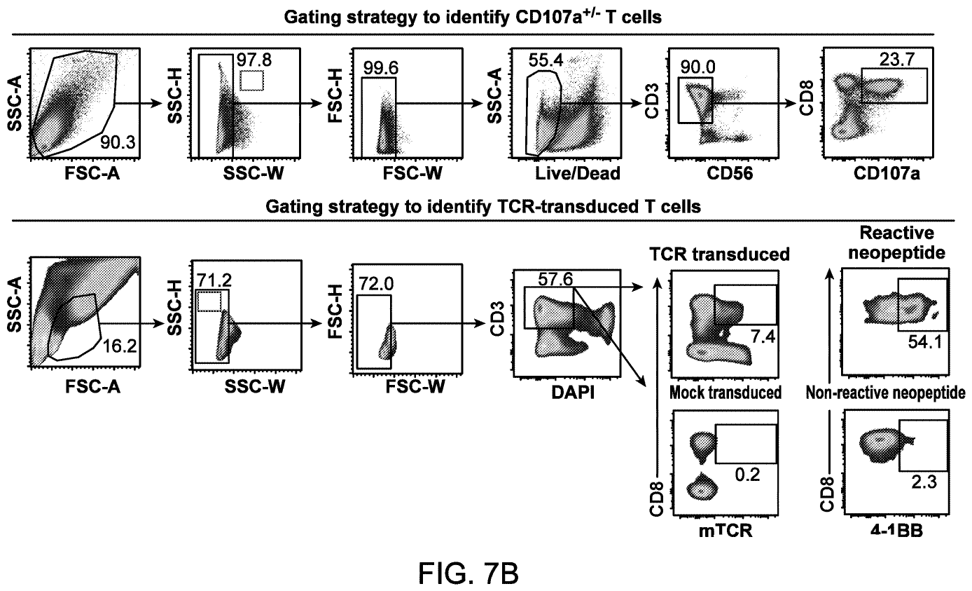

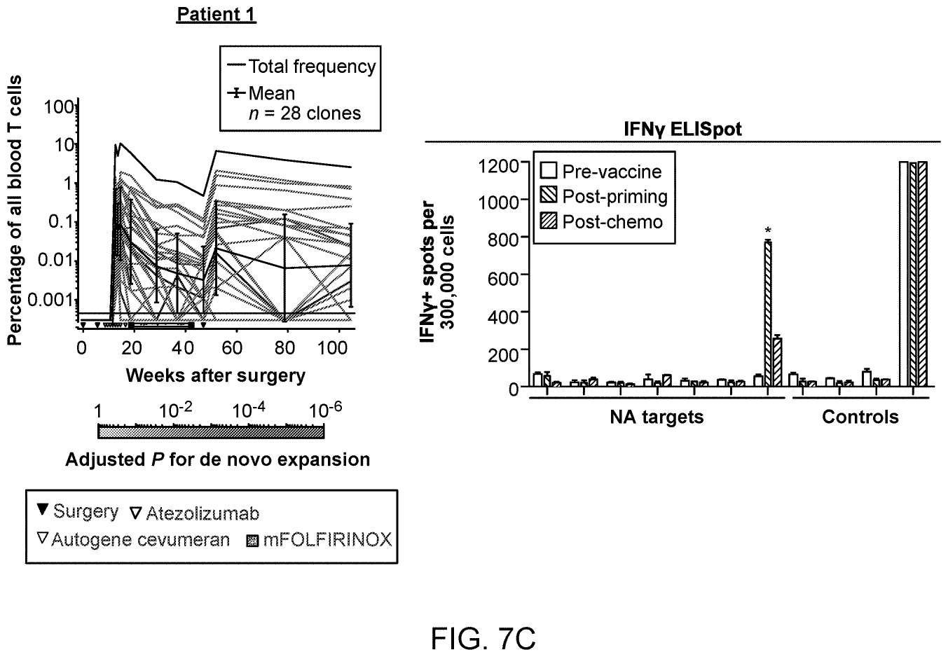

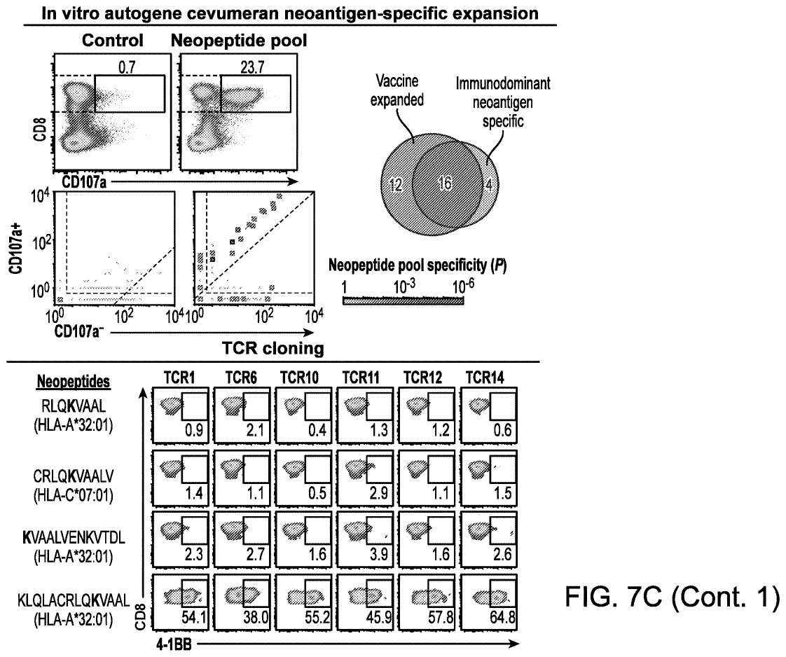

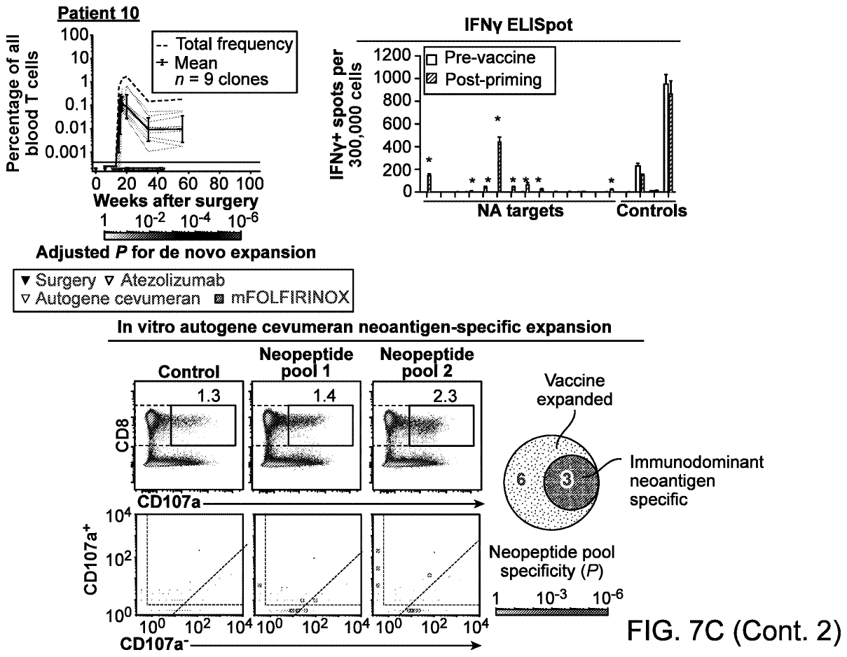

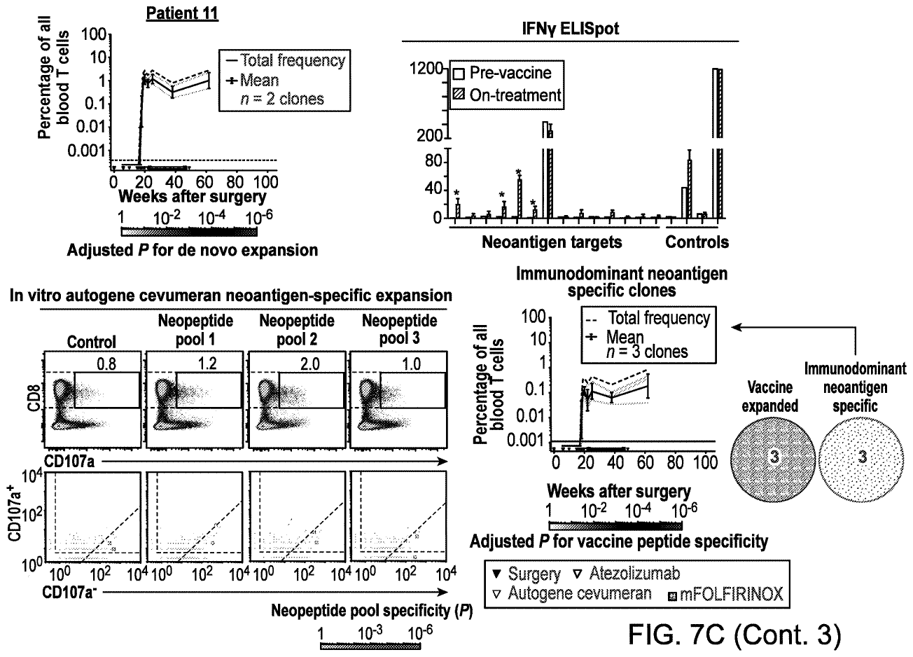

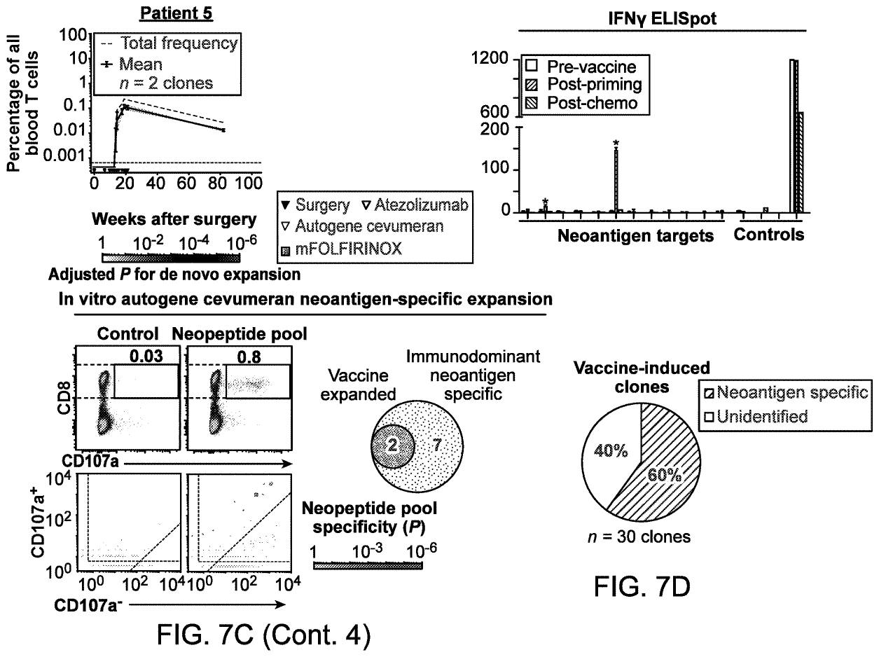

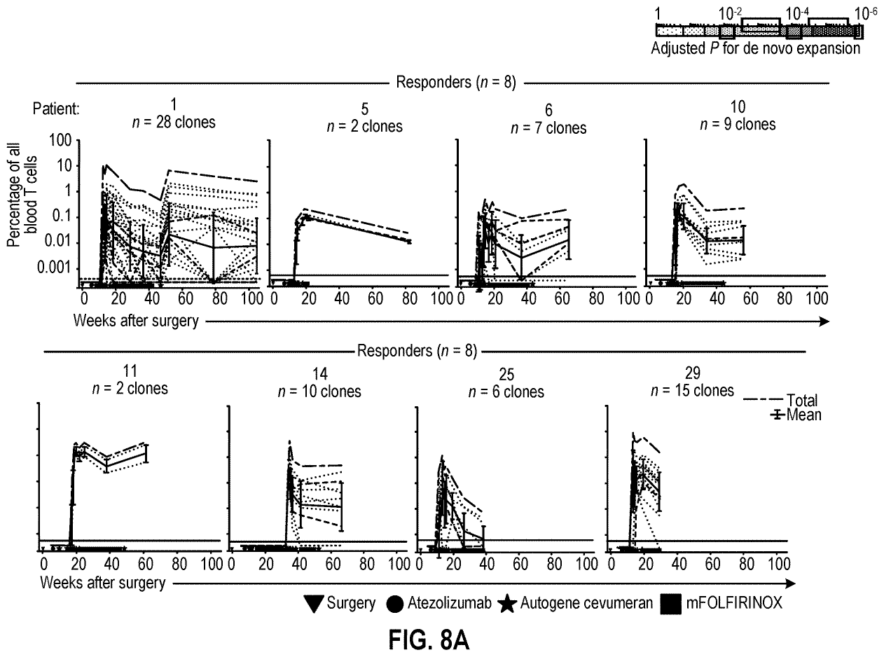

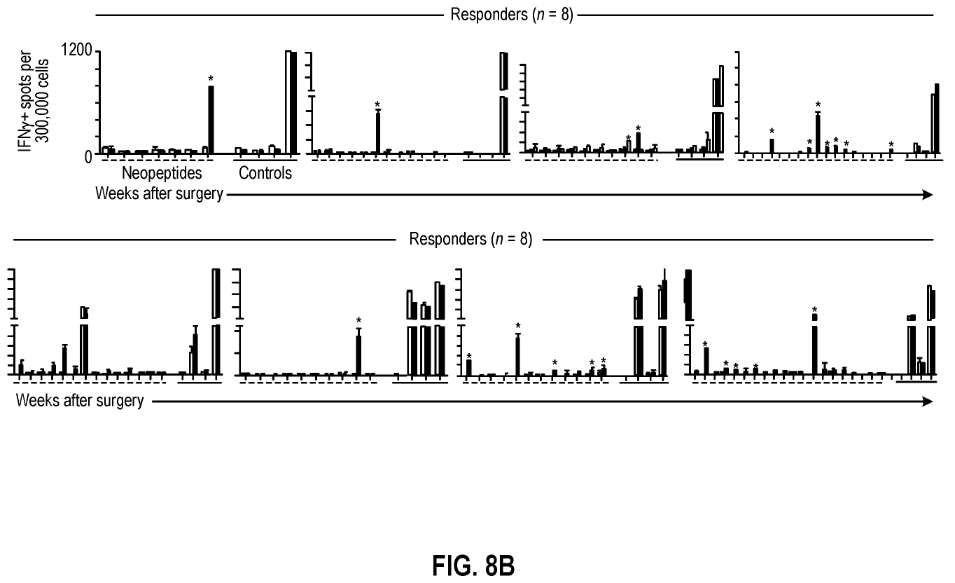

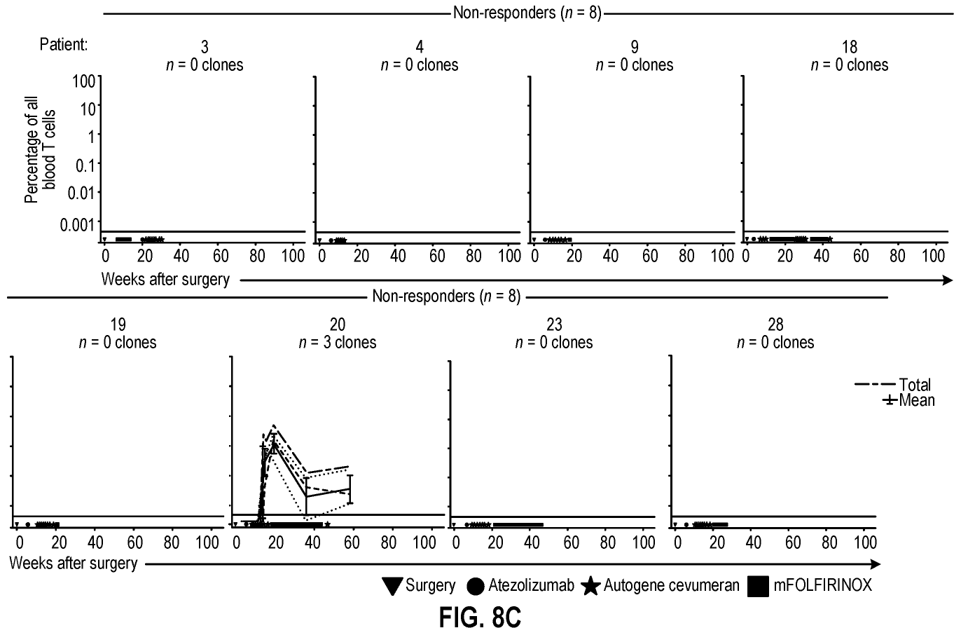

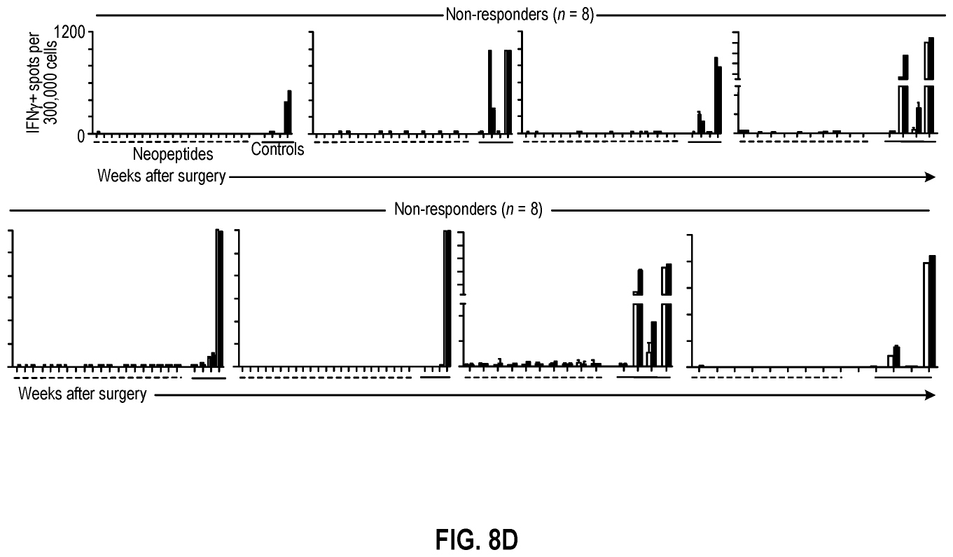

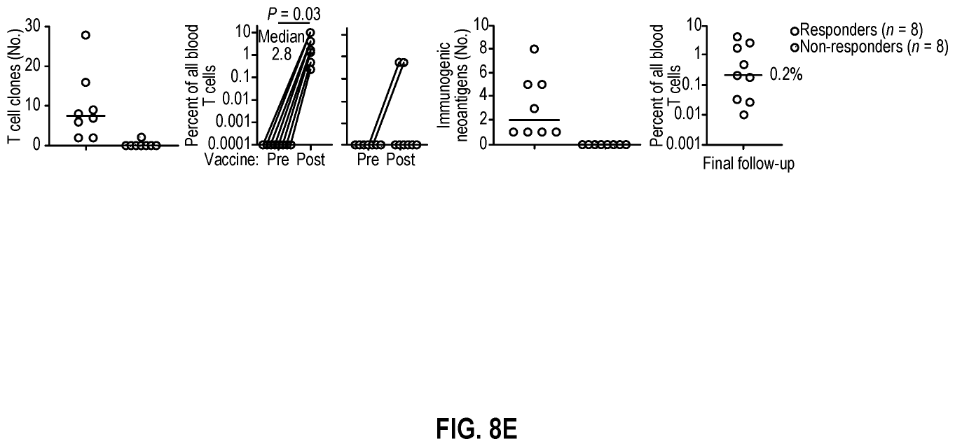

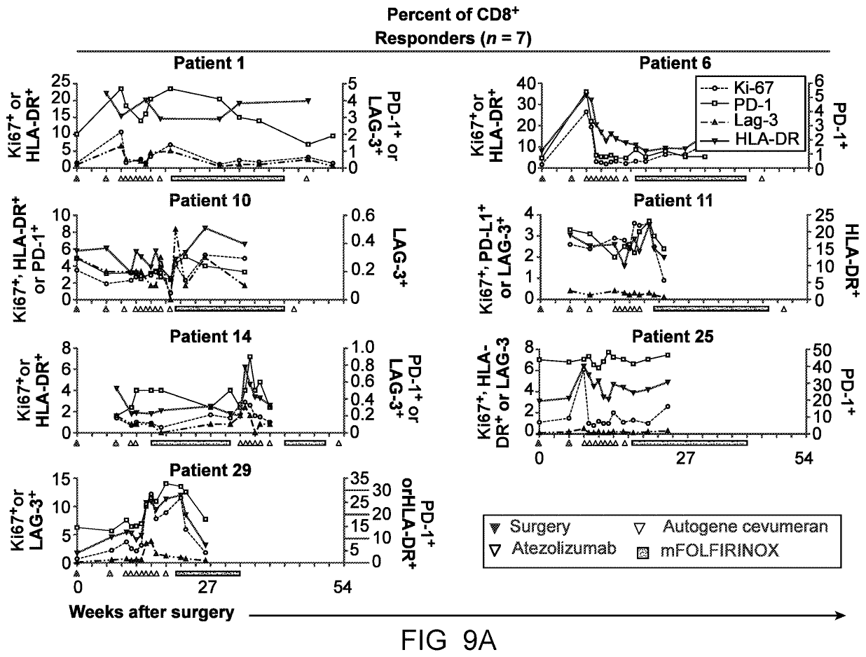

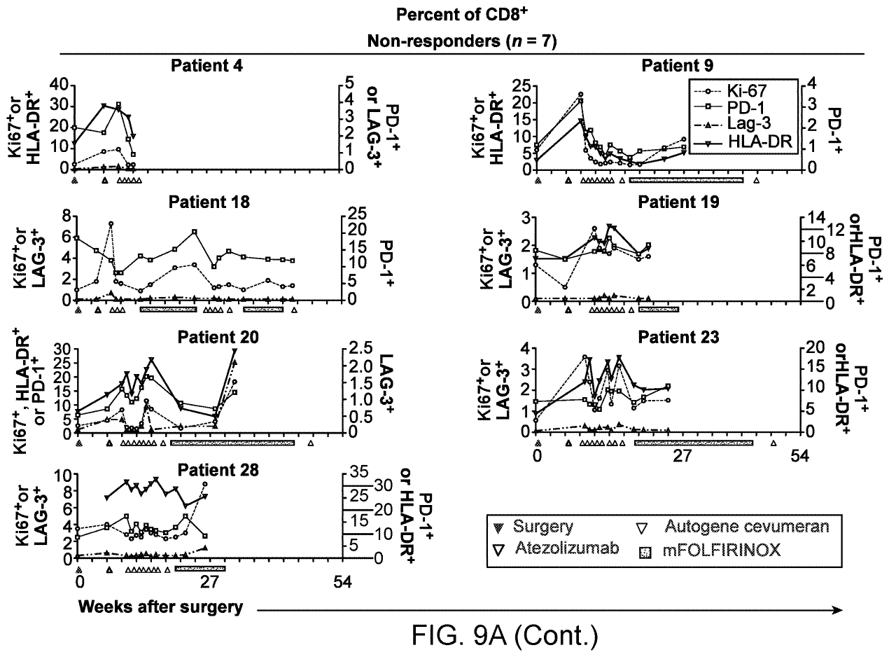

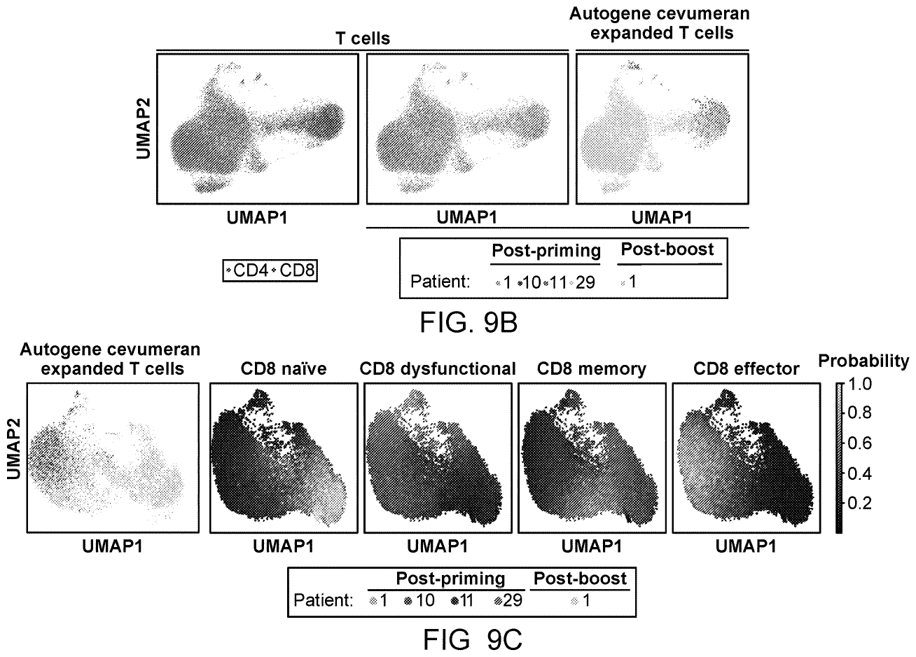

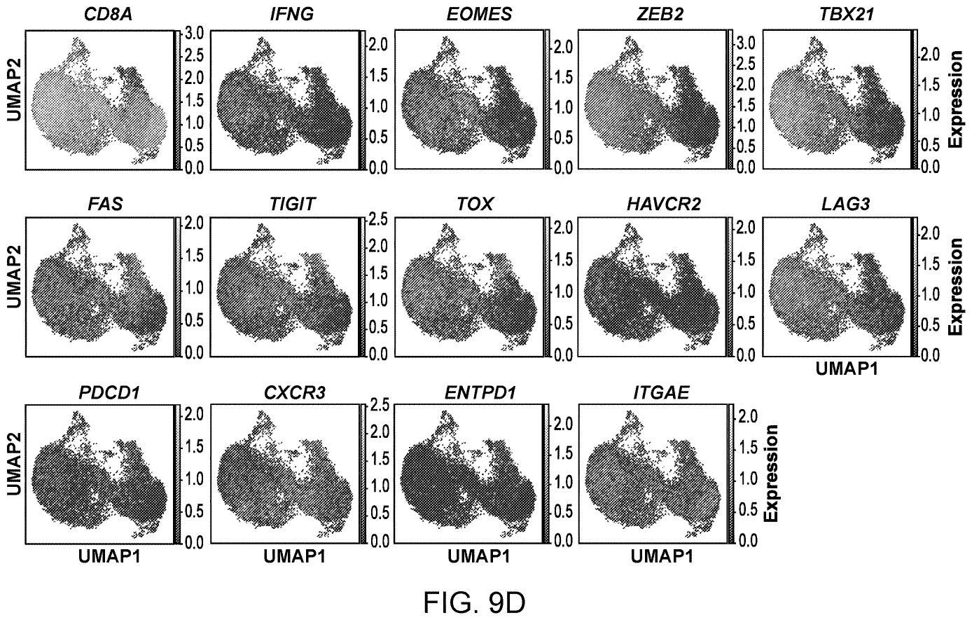

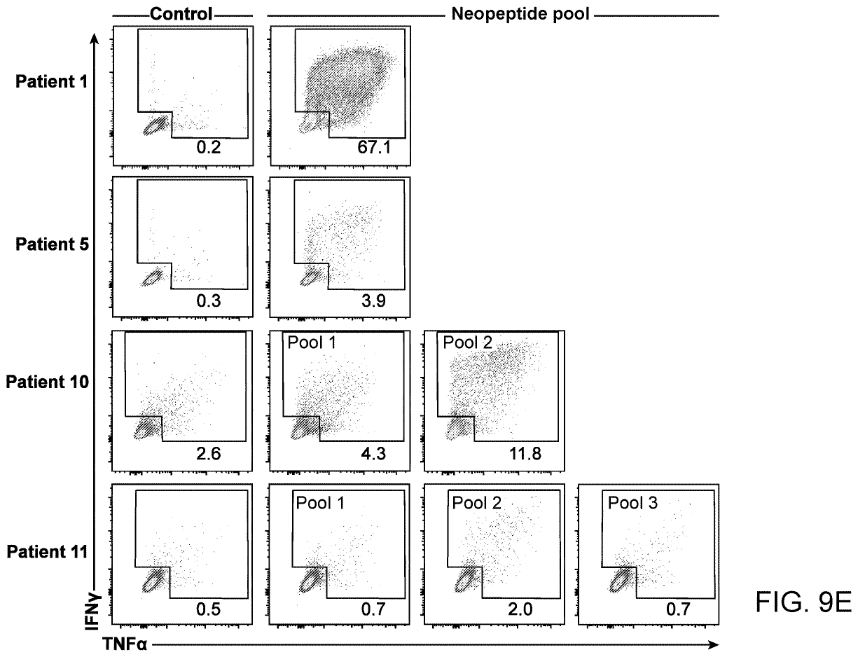

Autogene cevumeran induced immune responses in 8 of 16 (50%) vaccinated patients (FIG. 2A; FIGS. 7A-7D) and expanded multiple T cell clones (median 7.5 clones) from undetectable levels to up to 10% (median 2.8%) of all blood T cells (FIG. 2B; FIG. 8A-8B). Autogene cevumeran-expanded T cells recognized multiple vaccine neoantigens (median 2) in 50% of immune responders (FIG. 2C; FIG. 8B). These autogene cevumeran-expanded CD8⁺ T cells (FIGS. 9A-9D) expressed lytic granules (granzyme B, perforin 1 FIG. 2D) and cytokines (IFNγ, TNFα; FIG. 9E), to resemble effector T cells induced by protective viral vaccines²⁵ (FIGS. 9C and 9D). Autogene cevumeran-expanded CD8⁺ T cell clones did not overlap with atezolizumab-expanded T cell clones (FIGS. 10A-10C) and persisted in the blood as high as 2.5% of all blood T cells up to 2 years after surgery (median 0.2% of all blood T cells) (FIG. 8E) despite subsequent mFOLFIRINOX.

### Vaccine Response and Recurrence

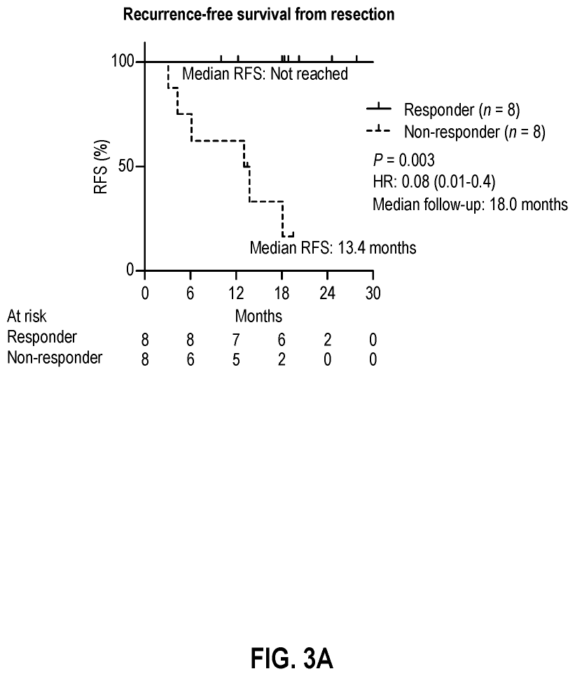

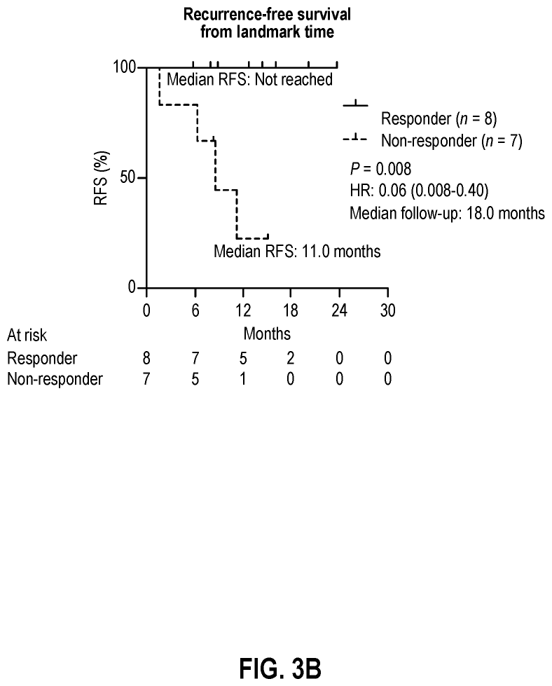

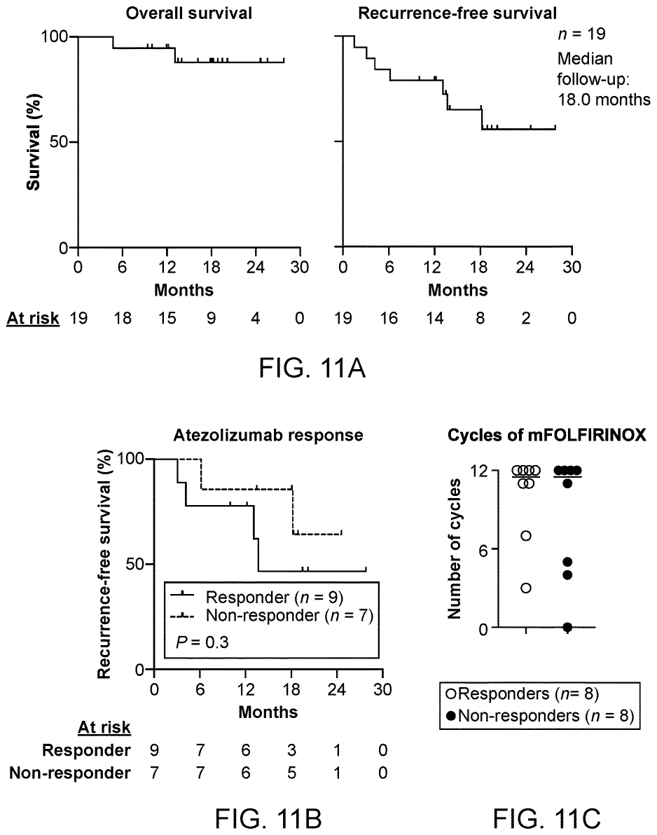

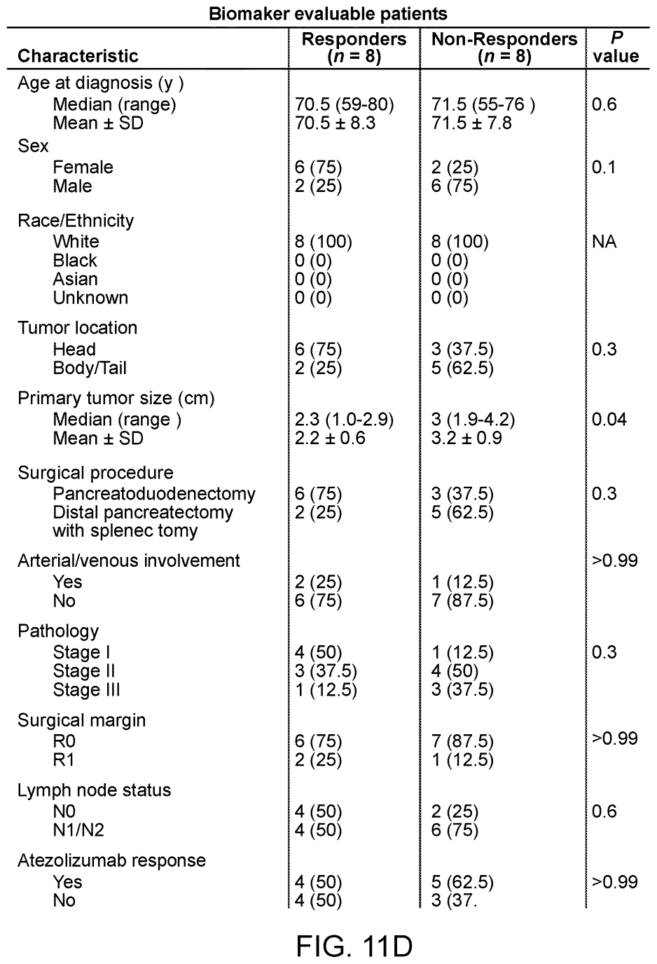

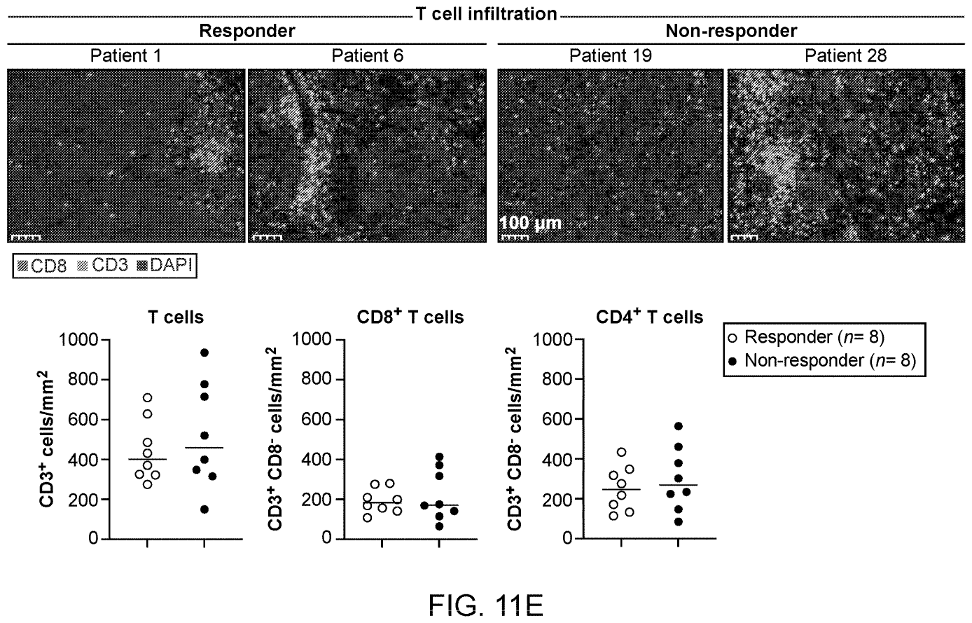

At a median follow-up of 18 months that extended beyond the prespecified secondary endpoint, 8 autogene cevumeran responders had a median RFS that was not reached, compared to the 8 non-responders who had a median RFS of 13.4 months (FIG. 3A; FIG. 11A). To exclude a time-to-response bias,²⁶ we performed a landmark analysis to correlate RFS to response in patients that were recurrence-free when completing all 8 autogene cevumeran priming doses (landmark RFS). Median landmark RFS was similarly not reached in responders compared to 11.0 months in non-responders (FIG. 3B). Response to atezolizumab, lymph node and margin positivity, the number of chemotherapy doses, and density of intratumoral T cells did not correlate with RFS (FIGS. 11B-11E).

### Vaccine Response and Neoantigen Quality

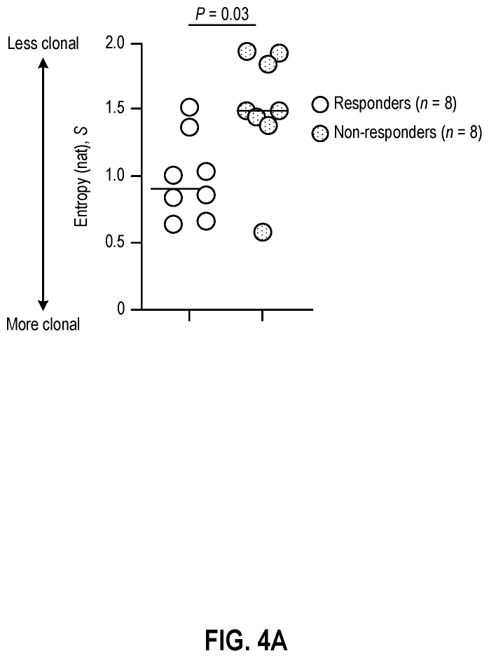

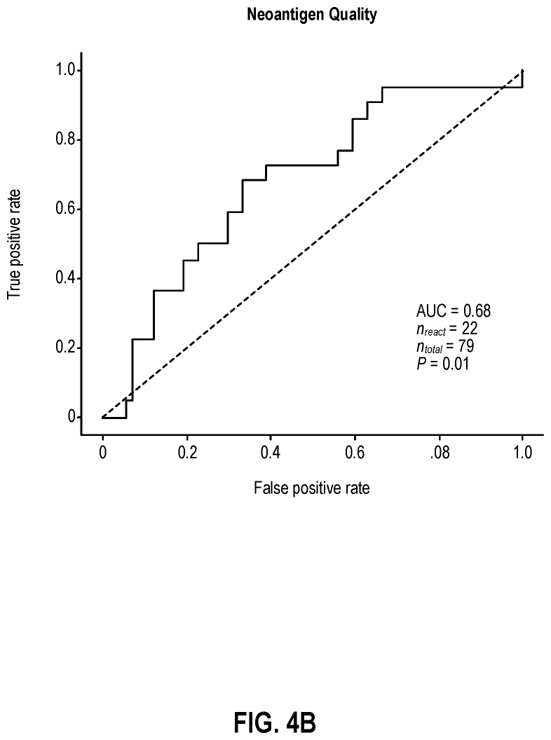

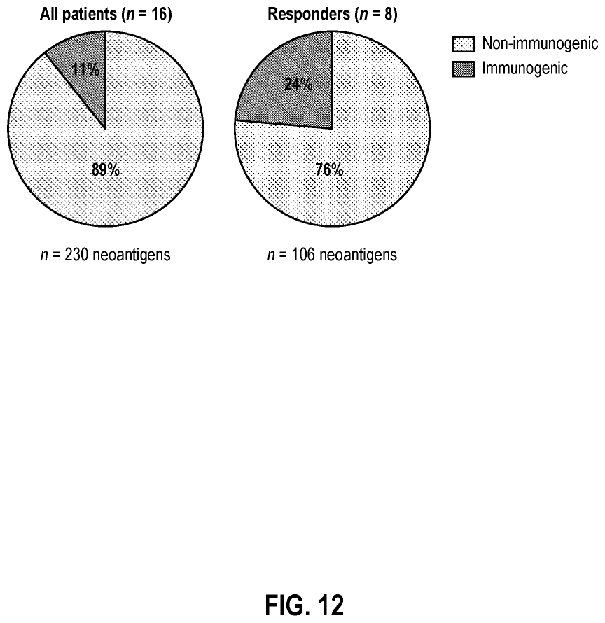

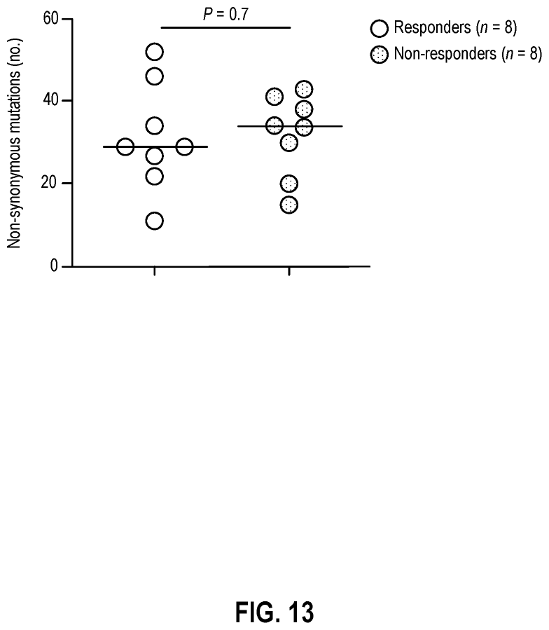

As autogene cevumeran expanded T cells specific to 25 of 106 neoantigens (24%) in responders (FIG. 12), we searched for biomarkers that predict vaccine response. Previously, we identified primary tumors enriched in CD8⁺ T cells are also enriched in immunogenic “high quality” neoantigens,^{11,12,27} distributed in greater proportions across tumor clones. We thus posited that tumor clonality and neoantigen quality could potentially identify optimal tumors and neoantigens for effective vaccines. Consistently, responders had more clonal tumors¹² (FIG. 4A). Furthermore, in immunologic responders, neoantigen quality further identified neoantigens that expanded neoantigen-specific T cells in patients compared to all vaccinated neoantigens (FIG. 4B). Notably, responders had a similar number of nonsynonymous mutations as non-responders (FIG. 13).

### Neoantigen-Specific Intrahepatic T Cells and a Micrometastasis

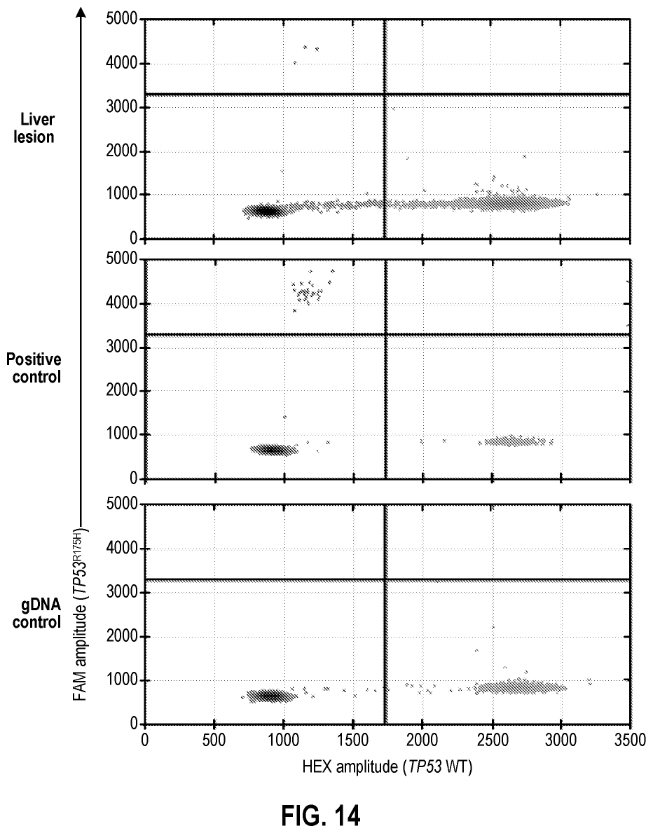

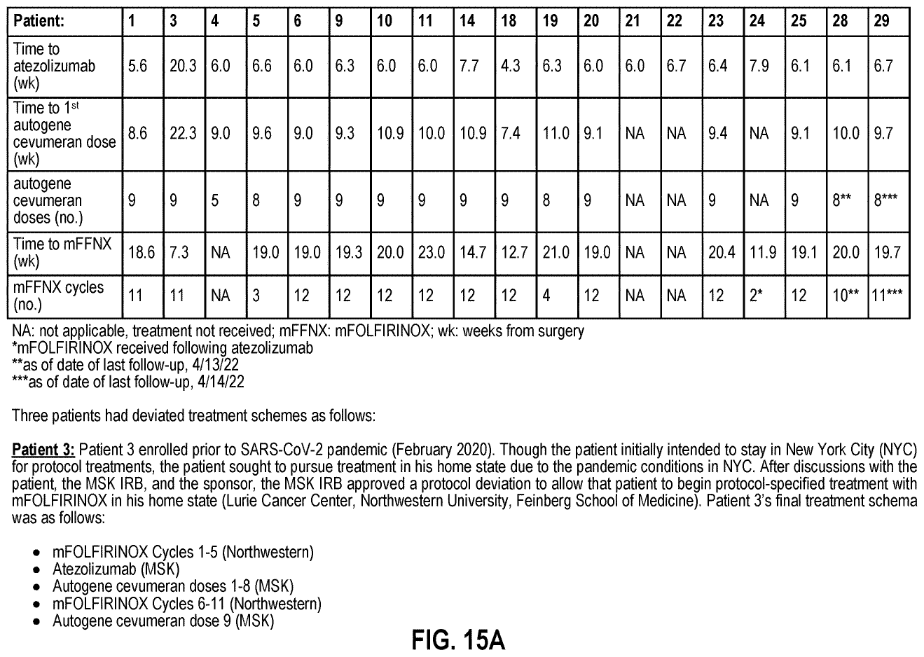

Without wishing to be bound by theory, adjuvant vaccines can expand neoantigen-specific T cells with the specificity and functionality to eradicate micrometastases, and delay recurrence. Patient 29, a responder with the second-highest maximal percentage of vaccine-induced T cells in the blood (FIGS. 8A-8E) developed a new, 7-mm liver lesion suspicious for a metastasis after vaccine priming (FIG. 5A). Biopsy revealed no malignant cells, but a dense lymphoid infiltrate that included all 15 peripheral blood T cell clones expanded by autogene cevumeran (FIG. 5B). Digital droplet PCR revealed this lymphoid infiltrate contained rare TP53^{R175H} mutated cells identical to the TP53^{R175H} driver mutation in this patient's primary tumor (FIG. 5C) (FIG. 14). This liver lesion disappeared on subsequent imaging (FIG. 5A), indicating neoantigen-specific T cells can possess the capacity to eradicate micrometastases.

### Discussion

We find that adjuvant autogene cevumeran, an individualized mRNA neoantigen vaccine, in combination with atezolizumab and mFOLFIRINOX, is safe, feasible, and generates substantial neoantigen-specific T cell activity in 50% of unselected PDAC patients. Vaccine-induced T cells are durable and persist up to 2 years despite post-vaccination chemotherapy. Vaccine-induced T cell responses correlate with delayed PDAC recurrence. Despite the limited sample size, these early results are encouraging given all current PDAC therapies are largely ineffective. With PDAC deaths projected to reach the second-highest cause of cancer death in 3 years,²⁸ larger studies of individualized mRNA neoantigen vaccines in PDAC should be urgently initiated.

Though one prior study proved that individualized mRNA vaccines can activate T cells against neoantigens in humans,¹⁵ this study was in melanoma—a highly mutated cancer with many neoantigens, and most patients exhibiting a T cell rich immune inflamed phenotype—that is evidently sensitive to multiple different immunotherapies.²⁹ The current urgent need for new immunotherapies remains for the majority of patients whose tumors are not inflamed, but rather exhibit immune excluded or desert phenotypes and are largely insensitive to current immunotherapies. Indeed, the prevailing thought has been that the low passenger mutation rate in cold tumors likely renders such tumors with insufficient neoantigens for vaccines. Thus, we notably report the first evidence that despite its low mutation rate, an mRNA vaccine can provoke T cell activity against neoantigens in PDAC, which predominantly displays stroma-rich immune excluded or desert phenotypes. Whether individualized mRNA neoantigen vaccines can similarly activate T cells in unfavorable immune phenotypes of other cancers should now be more broadly tested.

It is notable that autogene cevumeran responders had more clonal tumors. As more clonal tumors were a feature of immunogenic PDACs in long-term survivors—possibly representing a tumor in the earlier stages of immune editing¹²—a more clonal primary tumor may indicate the immune system's ability to recognize a tumor, and thus respond to the tumor's vaccine. Notwithstanding, a clonal tumor may also simplify neoantigen selection by creating a more genetically homogeneous tumor of malignant cells, much like a homogeneous clade of viruses. It is also notable that neoantigen quality^{11,12,27}—a framework to select immunogenic neoantigens—can discriminate immunogenic from non-immunogenic vaccine neoantigens, indicating neoantigen quality may allow rational selection of immunogenic neoantigens for vaccines.

A deterrent to personalized cancer vaccination has remained whether the complex process to individually analyze tumors and custom synthesize vaccines can be scaled to rapidly and reliably deliver vaccines in clinically relevant time frames. This is pertinent as parallel efforts to manufacture neoantigen vaccines with peptides³⁰ have taken ˜18-20 weeks, a time frame not compatible with fast-paced oncology clinics. Our study outlines that mRNA neoantigen vaccines can be individualized in 9 weeks, even for a genomically more “difficult” tumor with higher stromal content, where the sensitivity of data science pipelines to accurately detect neoantigens in real-time has remained in question. We further demonstrate that individualized vaccination can be fully integrated into a standard clinical workflow even after complex oncologic surgery. Thus, though the first experience with individualized cancer vaccines predated the COVID-19 pandemic,¹⁵ the recent realization that mRNA can make vaccines for a new virus at remarkable speeds leave the field poised to test if mRNA technology can further accelerate vaccine manufacture times to rapidly deploy individualized cancer vaccines.

We tested individualized mRNA vaccines in the adjuvant cancer setting motivated by observations that vaccines against pathogens have historically demonstrated the greatest efficacy in preventative and not therapeutic settings. Without wishing to be bound by theory, this likely reflects that an effective vaccine requires an optimally functioning host immune system. In the current study, the marginal over-representation of splenectomized patients in non-responders (FIG. 11D) may reflect how such impaired host immunity may blunt a vaccine response, particularly as lipoplexes preferentially target splenic antigen-presenting cells.³¹ As advanced cancer patients likely have more global impairments in host immunity, it is reasonable to posit that cancer vaccines as monotherapies will be most effective in patients with minimal residual disease. Further knowledge gaps on neoantigen heterogeneity between tumor sites hamper optimal neoantigen selection in advanced cancer. Thus, testing vaccines in the adjuvant setting, uncovering biomarkers of drug activity, and then applying vaccines in the advanced setting. This approach may be broadly appropriate for drugs that require host immunologic health to function.

Overall, we report preliminary evidence that adjuvant autogene cevumeran, an individualized mRNA neoantigen vaccine, in combination with atezolizumab, and mFOLFIRINOX induces substantial T cell activity in surgically resected PDAC patients that correlates with delayed recurrence. A follow-up randomized trial is planned.

### REFERENCES CITED IN THIS EXAMPLE

1. Park, W., Chawla, A. & O'Reilly, E. M. Pancreatic Cancer: A Review. Jama 326, 851 (2021).
2. Sung, H. et al. Global Cancer Statistics 2020: GLOBOCAN Estimates of Incidence and Mortality Worldwide for 36 Cancers in 185 Countries. Ca Cancer J Clin 71, 209-249 (2020).
3. Siegel, R. L., Miller, K. D., Fuchs, H. E. & Jemal, A. Cancer Statistics, 2021. Ca Cancer J Clin 71, 7-33 (2021).
4. Conroy, T. et al. FOLFIRINOX versus Gemcitabine for Metastatic Pancreatic Cancer. New Engl J Med 364, 1817-1825 (2011).
5. Hoff, D. D. V. et al. Increased Survival in Pancreatic Cancer with nab-Paclitaxel plus Gemcitabine. New Engl J Med 369, 1691-1703 (2013).
6. Philip, P. A. et al. Nab-paclitaxel plus gemcitabine in patients with locally advanced pancreatic cancer (LAPACT): a multicentre, open-label phase 2 study. Lancet Gastroenterology Hepatology 5, 285-294 (2019).
7. Strobel, O., Neoptolemos, J., Jäger, D. & Buchler, M. W. Optimizing the outcomes of pancreatic cancer surgery. Nat Rev Clin Oncol 16, 11-26 (2018).
8. Royal, R. E. et al. Phase 2 trial of single agent Ipilimumab (anti-CTLA-4) for locally advanced or metastatic pancreatic adenocarcinoma. Journal of immunotherapy (Hagerstown, Md: 1997) 33, 828-833 (2010).
9. Yarchoan, M., Hopkins, A. & Jaffee, E. M. Tumor Mutational Burden and Response Rate to PD-1 Inhibition. New Engl J Medicine 377, 2500-2501 (2017)
10. Bailey, P. et al. Exploiting the neoantigen landscape for immunotherapy of pancreatic ductal adenocarcinoma. Sci Rep-uk 6, 35848 (2016).
11. Balachandran, V. P. et al. Identification of unique neoantigen qualities in long-term survivors of pancreatic cancer. Nature 551, 512-516 (2017).
12. Luksza, M. et al. Neoantigen quality predicts immunoediting in survivors of pancreatic cancer. Nature 606, 389-395 (2022).
13. Schumacher, T. N. & Schreiber, R. D. Neoantigens in cancer immunotherapy. Science 348, 69-74 (2015).
14. Vormehr, M., Türeci, Ö. & Sahin, U. Harnessing Tumor Mutations for Truly Individualized Cancer Vaccines. Annu Rev Med 70, 395-407 (2019).
15. Sahin, U. et al. Personalized RNA mutanome vaccines mobilize poly-specific therapeutic immunity against cancer. Nature 547, 222-226 (2017).
16. Heil, F. et al. Species-Specific Recognition of Single-Stranded RNA via Toll-like Receptor 7 and 8. Science 303, 1526-1529 (2004).
17. Diebold, S. S., Kaisho, T., Hemmi, H., Akira, S. & Sousa, C. R. e. Innate Antiviral Responses by Means of TLR7-Mediated Recognition of Single-Stranded RNA. Science 303, 1529-1531 (2004).
18. Lund, J. M. et al. Recognition of single-stranded RNA viruses by Toll-like receptor 7. Proc National Acad Sci 101, 5598-5603 (2004).
19. Sahin, U. et al. An RNA vaccine drives immunity in checkpoint-inhibitor-treated melanoma. Nature 585, 107-112 (2019).
20. Kreiter, S. et al. Mutant MHC class II epitopes drive therapeutic immune responses to cancer. Nature 520, 692-6 (2014).
21. Braiteh, F. et al. Abstract CT169: A phase Ia study to evaluate R07198457, an individualized Neoantigen Specific immunoTherapy (iNeST), in patients with locally advanced or metastatic solid tumors. Cancer Res 80, CT169-CT169 (2020).
22. Lopez, J. S. et al. Abstract CT301: A phase Ib study to evaluate R07198457, an individualized Neoantigen Specific immunoTherapy (iNeST), in combination with atezolizumab in patients with locally advanced or metastatic solid tumors. Cancer Res 80, CT301-CT301 (2020).
23. Dindo, D., Demartines, N. & Clavien, P.-A. Classification of Surgical Complications: A New Proposal With Evaluation in a Cohort of 6336 Patients and Results of a Survey. Ann Surg 240, 205-213 (2004).
24. Kranz, L. M. et al. Systemic RNA delivery to dendritic cells exploits antiviral defence for cancer immunotherapy. Nature 534, 396-401 (2016).
25. Akondy, R. S. et al. Origin and differentiation of human memory CD8 T cells after vaccination. Nature 552, 362-367 (2015).
26. Anderson, J. R., Cain, K. C. & Gelber, R. D. Analysis of survival by tumor response. J Clin Oncol Official J Am Soc Clin Oncol 1, 710-9 (1983).
27. Luksza, M. et al. A neoantigen fitness model predicts tumour response to checkpoint blockade immunotherapy. Nature 551, 517-520 (2017).
28. L., R. et al. Projecting cancer incidence and deaths to 2030: the unexpected burden of thyroid, liver, and pancreas cancers in the United States. Cancer research 74, 2913-21 (2014).
29. Carlino, M. S., Larkin, J. & Long, G. V. Immune checkpoint inhibitors in melanoma. Lancet Lond Engl 398, 1002-1014 (2020).
30. Ott, P. A. et al. An immunogenic personal neoantigen vaccine for patients with melanoma. Nature 547, 217-221 (2017).
31. Kranz, L. M. et al. Systemic RNA delivery to dendritic cells exploits antiviral defence for cancer immunotherapy. Nature 534, 396-401 (2016).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method of determining a likelihood of responsiveness to an immunotherapy in a subject suffering from cancer, comprising:
receiving, by one or more processors, a dataset identifying a plurality of sequence reads, each sequence read of the plurality sequence reads corresponding to a respective T-cell receptor beta locus (TRB) sequence or T-cell receptor alpha locus (TRA) sequence derived from T-cell receptor (TCR) encoding polynucleotides present in a first plurality of T cell clones in a first biological sample from the subject administered with an immunotherapy, wherein the first plurality of T cell clones belong to a plurality of clonotypes;
for each clonotype of the plurality of clonotypes:
identifying, by the one or more processors, a first distribution of the first plurality of T cell clones belonging to the clonotype in the first biological sample using the plurality of sequence reads from the dataset;
comparing, by the one or more processors, the first distribution of the first plurality of T cell clones belonging to the clonotype with a second distribution of a second plurality of T cells belonging to the clonotype in a reference biological sample;
generating, by the one or more processors, a first significance value indicating an expansion of T cell clones of the clonotype within the first biological sample based on comparing the first distribution with the second distribution in accordance with a Fisher's exact test;
adjusting, by the one or more processors, the first significance value for the clonotype based on a number of clonotypes within the plurality of clonotypes in the first biological sample and the reference biological sample, to generate a second significance value;
determining, by the one or more processors, a score indicating a likelihood of responsiveness to the immunotherapy in the subject based on the second significance value for at least one clonotype of the plurality of clonotypes; and
storing, by the one or more processors, using one or more data structure, an association between the score indicating the likelihood of responsiveness and the subject optionally wherein adjusting the significance value further comprises adjusting the significance value for each clonotype of the plurality of clonotypes in accordance with a Bonferroni correction based on a number of the plurality of clonotypes in both the first biological sample and the reference biological sample.

2. The method of claim 1, further comprising:
(A)
determining, by the one or more processors, a threshold to compare against the score based on a frequency of the second plurality of T cells corresponding to each clonotype of the plurality of clonotypes in the reference biological sample: or
(B)
identifying, by the one or more processors, the subject as a responder to the immunotherapy based on the score satisfying a threshold; and
providing, by the one or more processors, an instruction to continue administration of the immunotherapy to the subject, responsive to identifying the subject as the responder; or
(C)
identifying, by the one or more processors, the subject as not a responder to the immunotherapy based on the score not satisfying a threshold; and
providing, by the one or more processors, an instruction to discontinue administration of the immunotherapy to the subject, responsive to identifying the subject as the non-responder.

3. (canceled)

4. (canceled)

5. The method of claim 1, wherein generating the significance value further comprises generating, for each clonotype of the plurality of clonotypes, the significance value indicating a two-fold expansion of T cell clones.

6. The method of claim 5, further comprising
rescaling, by the one or more processors, for each clonotype of the plurality of clonotypes, the first distribution of the first plurality of T cell clones of the clonotype based on a difference between (i) a factor of a number of the plurality of clonotypes in the first biological sample and (ii) a number of the first plurality of T cell clones belonging to the clonotype; or
rescaling, by the one or more processors, for each clonotype of the plurality of clonotypes, the second distribution of the second plurality of T cells of the clonotype to compare against the first distribution of T cells of the clonotype, based on a difference between (i) a number of the plurality of clonotypes in the reference biological sample and (ii) a number of the second plurality of T cell clones belonging to the clonotype.

7. (canceled)

8. (canceled)

9. The method of claim 1, further comprising:
determining, by the one or more processors, that the first distribution of the plurality of T cell clones belonging to at least one clonotype of the plurality of clonotypes does not satisfy a threshold; and
assigning, by the one or more processor, the first distribution of the plurality of T cell clones belonging to the at least one clonotype to a value based on a factor of a number of the plurality of clonotypes in the first biological sample;
and/or
removing, by the one or more processors, from the plurality of sequence reads, a subset of sequence reads determined as non-productive based on an identification of recombined TCR alpha CDR3 nucleotide sequences or TCR beta CDR3 nucleotide sequences from silenced alleles.

10. The method of claim 1, wherein identifying first distribution of T cell clones further comprises identifying, for each clonotype of the plurality of clonotypes, the first distribution of the first plurality of T cell clones corresponding to the clonotype based on a respective permutation of variable (V) gene, a joining (J) gene, and a nucleotide CDR3 sequence; or
wherein the reference biological sample is obtained from the subject prior to the administration of the immunotherapy; or
wherein the first biological sample and the second biological sample are the same type; or
wherein the reference biological sample is the same type as the first biological sample and the second biological sample; or
wherein the immunotherapy comprises an anti-cancer vaccine, monoclonal antibody-based immunotherapy, or an immune checkpoint inhibitor; or
wherein the first biological sample is obtained at least 1 week, at least 2 weeks, at least 3 weeks, 4 at least weeks, or at least 5 weeks after the immunotherapy has been administered to the subject.

11. (canceled)

12. (canceled)

13. (canceled)

14. (canceled)

15. (canceled)

16. A method of monitoring responsiveness to at least one type of immunotherapy in a subject suffering from cancer, comprising:
receiving, by one or more processors, at a first time prior to an administration of the first immunotherapy to the subject, a first dataset identifying a first plurality of sequence reads, each sequence read of the first plurality sequence reads corresponding to a respective T-cell receptor beta locus (TRB) sequence or T-cell receptor alpha locus (TRA) sequence derived from T-cell receptor (TCR) encoding polynucleotides present in a first plurality of T cell clones in a first biological sample from the subject, wherein the first plurality of T cell clones belong to a plurality of clonotypes;
identifying, by the one or more processors, for each of the plurality of clonotypes, a first distribution of the first plurality of T cell clones belonging to the clonotype in the first biological sample using the first plurality of sequence reads from the first dataset;
receiving, by the one or more processors, at a second time subsequent to the administration of the first immunotherapy to the subject, a second dataset identifying a second plurality of sequence reads, each sequence read of the second plurality sequence reads corresponding to a respective TRB sequence or TRA sequence derived from TCR encoding polynucleotides present in a second plurality of T cell clones in a second biological sample from the subject;
for each clonotype of the plurality of clonotypes:
identifying, by the one or more processors, a second distribution of the second plurality of T cell clones belonging to the clonotype in the second biological sample using the second plurality of sequence reads from the dataset;
comparing, by the one or more processors, the first distribution of the first plurality of T cell clones with the second distribution of the second plurality of T cells;
generating, by the one or more processors, a first significance value indicating an expansion of T cell clones of the clonotype within the second biological sample based on comparing the first distribution with the second distribution in accordance with a Fisher's exact test;
adjusting, by the one or more processors, the first significance value for the clonotype based on a number of clonotypes within a plurality of clonotypes in the first biological sample and the second biological sample, to generate a second significance value;
determining, by the one or more processors, a score identifying a degree of responsiveness to the first immunotherapy in the subject over the first time and the second time based on the second significance value for at least one of the plurality of clonotypes; and
identifying, by the one or more processors, the subject as one of a responder or a non-responder to the first immunotherapy based on the score satisfying a threshold, optionally wherein generating the first significance value further comprises generating for each clonotype of the plurality of clonotypes, the first significance value indicating a two-fold expansion of T cell clones.

17. The method of claim 16, further comprising:
receiving, by the one or more processors, at a third time prior to an administration of a second immunotherapy to the subject, a third dataset identifying a third plurality of sequence reads, each sequence read of the third plurality sequence reads corresponding to a respective TRB sequence or TRA sequence derived from TCR encoding polynucleotides present in a third plurality of T cell clones in a third biological sample from the subject;
identifying, by the one or more processors, for each clonotype of the plurality of clonotypes, a third distribution of the third plurality of T cell clones belonging to the clonotype in the third biological sample using the third plurality of sequence reads from the third dataset;
receiving, by the one or more processors, at a fourth time subsequent to the administration of the second immunotherapy to the subject, a fourth dataset identifying a fourth plurality of sequence reads, each sequence read of the fourth plurality sequence reads corresponding to a respective TRB sequence or TRA sequence derived from TCR encoding polynucleotides present in a fourth plurality of T cell clones in a fourth biological sample from the subject;
for each clonotype of the plurality of clonotypes in the fourth biological sample:
identifying, by the one or more processors, a fourth distribution of the fourth plurality of T cell clones belonging to the clonotype in the second biological sample using the fourth plurality of sequence reads from the dataset;
comparing, by the one or more processors, the third distribution of the third plurality of T cell clones with the fourth distribution of the fourth plurality of T cells;
generating, by the one or more processors, a second significance value indicating an expansion of T cell clones of the clonotype within the fourth biological sample based on comparing the third distribution with the fourth distribution in accordance with a Fisher's exact test;
adjusting, by the one or more processors, the third significance value for the clonotype based on a number of clonotypes within a second plurality of clonotypes in the third biological sample and the fourth biological sample, to generate a fourth significance value;
determining, by the one or more processors, a second score identifying a second degree of responsiveness to the second immunotherapy in the subject over the third time and the fourth time based on the fourth significance value for the clonotype; and
identifying, by the one or more processors, the subject as one of a responder or a non-responder to the second immunotherapy based on the second score satisfying a second threshold, wherein the first immunotherapy and the second immunotherapy are distinct types.

18. The method of claim 17, further comprising selecting, by the one or more processors, an instruction from a plurality of instructions based on identifying the subject as one of the responder or the non-responder to the first immunotherapy or the second immunotherapy, the plurality of instructions including:
a first instruction to continue administering the first immunotherapy responsive to identifying the subject as the responder to the first immunotherapy,
a second instruction to discontinue administering the first immunotherapy responsive to identifying the subject as the non-responder to the first immunotherapy,
a third instruction to continue administering the second immunotherapy responsive to identifying the subject as the responder to the second immunotherapy,
a fourth instruction to discontinue administering the second immunotherapy responsive to identifying the subject as the non-responder to the second immunotherapy,
a fifth instruction to continue administering at least one of the first immunotherapy and the second immunotherapy, responsive to identifying the subject as the responder to the first immunotherapy and the second immunotherapy, and
a sixth instruction to discontinue administering both the first immunotherapy and the second immunotherapy, responsive to identifying the subject as the non-responder to the first immunotherapy and the second immunotherapy.

19. The method of claim 17, further comprising:
comparing, by the one or more processors, the score to a first threshold and the second score to a second threshold; and
identifying, by the one or more processors, the subject as the responder to the first immunotherapy, responsive to the score satisfying the first threshold;
identifying, by the one or more processors, the subject as the non-responder to the first immunotherapy, responsive to the score not satisfying the first threshold;
identifying, by the one or more processors, the subject as the responder to the second immunotherapy, responsive to the second score satisfying the second threshold;
identifying, by the one or more processors, the subject as the non-responder to the second immunotherapy, responsive to the second score not satisfying the second threshold.

20. The method of claim 17, further comprising:
determining, by the one or more processors, the first threshold to compare against the score based on a first frequency of the first plurality of T cells corresponding to each clonotype of the plurality of clonotypes in the first biological sample; or
determining, by the one or more processors, the second threshold to compare against the score based on a second frequency of the third plurality of T cells corresponding to each clonotype of the plurality of clonotypes in the third biological sample.

21. (canceled)

22. The method of claim 17, further comprising:
rescaling, by the one or more processors, for each clonotype of the plurality of clonotypes in the second biological sample, the second distribution of the second plurality of T cell clones of the clonotype based on a first difference between (i) a factor of a first number of the plurality of clonotypes in the second biological sample and (ii) a first number of the second plurality of T cell clones belonging to the clonotype; or
rescaling, by the one or more processors, for each clonotype of the plurality of clonotypes in the fourth biological sample, the second distribution of the second plurality of T cell clones of the clonotype based on a second difference between (i) a factor of a second number of the plurality of clonotypes in the second biological sample and (ii) a second number of the fourth plurality of T cell clones belonging to the clonotype; or
rescaling, by the one or more processors, for each clonotype of the plurality of clonotypes in the second biological sample, the second distribution of the plurality of T cells of the clonotype to compare against the first distribution of T cells of the clonotype, based on a first difference between (i) a first number of the plurality of clonotypes in the first biological sample and (ii) a second number of the plurality of T cell clones belonging to the clonotype; or
rescaling, by the one or more processors, for each clonotype of the plurality of clonotypes in the fourth biological sample, the fourth distribution of the plurality of T cells of the clonotype to compare against the first distribution of T cells of the clonotype, based on a difference between (i) a third number of the plurality of clonotypes in the third biological sample and (ii) a fourth number of the plurality of T cell clones belonging to the clonotype.

23. (canceled)

24. The method of claim 17, wherein adjusting the first significance value further comprises adjusting the first significance value for each clonotype of the plurality of clonotypes in the second biological sample in accordance with a Bonferroni correction based on a number of the plurality of clonotypes in both the first biological sample and the second biological sample, or
wherein adjusting the second significance value further comprises adjusting the second significance value for each clonotype of the plurality of clonotypes in the fourth biological sample in accordance with a Bonferroni correction based on a number of the plurality of clonotypes in both the third biological sample and the fourth biological sample.

25. The method of claim 17, further comprising:
(A)
determining, by the one or more processors, that the second distribution of the second plurality of T cell clones belonging to at least one clonotype of the plurality of clonotypes does not satisfy a threshold; and
assigning, by the one or more processor, the second distribution of the second plurality of T cell clones belonging to the at least one clonotype to a value based on a factor of a number of the plurality of clonotypes in the second biological sample; or
(B)
determining, by the one or more processors, that the fourth distribution of the fourth plurality of T cell clones belonging to at least one clonotype of the plurality of clonotypes does not satisfy a threshold; and
assigning, by the one or more processor, the fourth distribution of the fourth plurality of T cell clones belonging to the at least one clonotype to a value based on a factor of a number of the plurality of clonotypes in the fourth biological sample.

26. (canceled)

27. The method of claim 17, further comprising:
removing, by the one or more processors, from each of the first plurality of sequence reads, second plurality of sequence reads, the third plurality of sequence reads, and the fourth plurality of sequence reads, a subset of sequence reads determined as non-productive based on an identification of recombined TCR alpha CDR3 nucleotide sequences or TCR beta CDR3 nucleotide sequences from silenced alleles.

28. The method of claim 17, wherein identifying the second distribution of T cell clones further comprises identifying, for each clonotype of the plurality of clonotypes, the second distribution of the second plurality of T cell clones corresponding to the clonotype based on a respective permutation of variable (V) gene, a joining (J) gene, and a nucleotide CDR3 sequence, or
wherein identifying the fourth distribution of T cell clones further comprises identifying, for each clonotype of the plurality of clonotypes, the fourth distribution of the fourth plurality of T cell clones corresponding to the clonotype based on a respective permutation of variable (V) gene, a joining (J) gene, and a nucleotide CDR3 sequence; or
wherein the first, second, third, and/or fourth biological sample are the same type; or
wherein the first immunotherapy or the second immunotherapy is an anti-cancer vaccine, monoclonal antibody-based immunotherapy, or an immune checkpoint inhibitor
wherein the second biological sample is obtained at least 1 week, at least 2 weeks, at least 3 weeks, 4 at least weeks, or at least 5 weeks after the first immunotherapy has been administered to the subject; or
wherein the fourth biological sample is obtained at least 1 week, at least 2 weeks, at least 3 weeks, 4 at least weeks, or at least 5 weeks after the second immunotherapy has been administered to the subject; or
wherein the second immunotherapy is administered at least at least 2 weeks, at least 3 weeks, 4 at least weeks, at least 5 weeks, at least 6 weeks, at least 7 weeks, at least 8 weeks, at least 9 weeks, at least 10 weeks, at least 11 weeks, at least 12 weeks, and at least 13 weeks after the first immunotherapy; or
wherein the first, second, third, and/or fourth plurality of T cell clones comprise one or more of CD4+ helper T cells, CD8+ cytotoxic T cells, CD8+ CD107+ T cells, central memory T cells, stem-cell-like memory T cells (or stem-like memory T cells), effector memory T cells, Natural killer T cells, Mucosal associated invariant T cells, and γδ T cells; or
wherein the first, second, third, and/or fourth biological sample comprises plasma, serum, whole blood, or PBMCs.

29. (canceled)

30. (canceled)

31. (canceled)

32. (canceled)

33. (canceled)

34. (canceled)

35. (canceled)

36. The method of claim 10, wherein the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, an anti-LAG-3 antibody, an anti-OX40 antibody, an anti-TIGIT antibody, an anti-B7-H3 antibody, an anti-B7-H4 antibody, and an anti-BTLA antibody; or
wherein the immune checkpoint inhibitor comprises pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189; or
wherein the anti-cancer vaccine comprises Individual Neoantigen-Specific Immunotherapy (iNeST), wherein the iNeST comprises autogene cevumeran; or
wherein the monoclonal antibody-based immunotherapy comprises an antibody, antigen binding fragment, or a derivative thereof; or
wherein the monoclonal antibody-based immunotherapy targets a tumor antigen, optionally wherein the tumor antigen is selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125, carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EDCAM, DLL3, PD-1, PD-L1, CD28, CD137, CD99, GloboH CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

37. (canceled)

38. (canceled)

39. (canceled)

40. (canceled)

41. (canceled)

42. The method of claim 1, wherein the cancer is selected from among carcinomas, sarcomas, hematopoietic cancers, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.
